(19) United States
(12) Patent Application Publication
(10) Pub. No.: US 2026/0085111 A1
(43) Pub. Date: Mar. 26, 2026

(54) SLIT2 RELATED COMPOSITIONS AND METHODS

(71) Applicant: EVEREST MEDICINES (SINGAPORE) PTE. LTD., Singapore (SG)

(72) Inventors: Jie ZHAO, Zhejiang (CN); Shan HE, Zhejiang (CN); Wei Jennifer YANG, Zhejiang (CN); He LI, Zhejiang (CN)

(73) Assignee: EVEREST MEDICINES (SINGAPORE) PTE. LTD., Singapore (SG)

(21) Appl. No.: 19/112,728

(22) PCT Filed: Sep. 18, 2023
(86) PCT No.: PCT/CN2023/119393

§ 371 Date: Mar. 18, 2025

Related U.S. Application Data
(63) Continuation-in-part of application No. PCT/CN2023/113804, filed on Aug. 18, 2023.
Application No. PCT/CN2023/113804 is a continuation-in-part of application No. PCT/CN2023/091590, filed on Apr. 28, 2023.
Application No. PCT/CN2023/091590 is a continuation-in-part of application No. PCT/CN2022/119873, filed on Sep. 20, 2022.

(51) Int. Cl. C07K 16/18; A61K 39/00; A61P 13/12

(52) U.S. Cl. CPC C07K 16/18; A61P 13/12; A61K 2039/505; C07K 2317/24; C07K 2317/52; C07K 2317/565; C07K 2317/92

(57) ABSTRACT

The antibodies bind to SLIT2 and related substitution mutants, including their manufacture and use.

### CROSS-REFERENCE TO RELATED APPLICATIONS

This application claims priority and benefit from PCT applications PCT/CN2022/119873, filed on Sep. 20, 2022; PCT/CN2023/091590, filed on Apr. 28, 2023; and PCT/CN2023/113804, filed on Aug. 18, 2023, the contents and disclosures of which are incorporated herein by reference in their entireties.

### BACKGROUND

Podocytes are specialized epithelial cells present in the outer surface of the glomerular basement membrane, which is part of the kidney glomerular filtration barrier. In the process of filtration, mechanisms involving adjacent podocytes provide filtration slits between cells bridged by a slit-diaphragm that forms the final barrier to protein permeation in the kidney. The interconnecting podocytes also adhere to the glomerular basement membrane, which is a dense network structure of secreted extracellular matrix molecules, through cell-matrix adhesion receptors such as integrins and dystroglycans. Glomerular filtration relies on normal podocyte adhesion at both the slit-diaphragm and the interface with the glomerular basement membrane, for example it is known that genetic mutations of slit-diaphragm proteins such as nephrin and podocyte-glomerular basement membrane adhesion proteins such as integrin α3 are associated with hereditary forms of nephrotic syndrome. Podocytes coordinate signals from cell junctions and cell-matrix interactions in response to environmental cues and provide for filtration regulation. Further, it has been shown that the slit-diaphragm protein nephrin regulates podocyte structure and function by promoting actin polymerization.

Slit2 is a secreted ligand for Robo1 and Robo2 receptors. Mutations in Slit2 and Robo2 lead to abnormalities of the kidney and related ureteric tract, which supports that this may be a requirement of this signaling pathway for kidney development. Studies in mouse knockouts supported a conclusion that Slit2-Robo2 signaling may restrict ureteric epithelium budding.

Roundabout Receptor 2 (ROBO2) is a single-pass transmembrane receptor for SLIT ligands. ROBO-SLIT pathway is originally characterized as repulsive guidance cues for axon pathfinding during the development of the nervous system. Recent studies have shown that ROBO2 also expresses on podocytes and is involved in regulating podocyte adhesion and migration.

Loss of normal kidney function affects a large percentage of the population, which has significant impacts on health and lifespan. Kidney diseases have historically been treated with generalized immunosuppressive agents, antihypertensives and diuretics with limited success. Such therapies have largely treated complications, symptoms or late manifestations of disease, and have had limited disease-modifying effects. Most known kidney disease is characterized by breakdown of the glomerular filtration barrier. As noted, specialized podocyte cells are known to play a key role in maintaining the glomerular filtration barrier.

The present invention addresses this and other related needs in the art.

### SUMMARY

In certain embodiments, provided herein are antibodies comprising: a heavy chain comprising complementarity determining regions (CDR) CDR-H1 comprising amino acid sequence of SEQ ID NO: 1; CDR-H2 comprising amino acid sequence of SEQ ID NO: 2; and CDR-H3 comprising amino acid sequence of SEQ ID NO: 3, respectively and having a specific binding activity to human SLIT2 and/or blocking activity to ROBO2; and a light chain comprising CDR-L1 comprising amino acid sequence of SEQ ID NO: 4 or 7; CDR-L2 comprising amino acid sequence of SEQ ID NO: 5; and CDR-L3 comprising amino acid sequence of SEQ ID NO: 6 and having a specific binding activity to human SLIT2 and/or blocking activity to ROBO2.

Also in frequently included embodiments according to the present disclosure, a recombinant antibody capable of modulating ROBO2 and SLIT2 interaction, wherein the antibody comprises: a heavy chain comprising complementarity determining regions (CDR) CDR-H1 comprising amino acid sequence of SEQ ID NO: 1; CDR-H2 comprising amino acid sequence of SEQ ID NO: 2; and CDR-H3 comprising amino acid sequence of SEQ ID NO: 3, respectively and having a specific binding activity to human SLIT2 and/or blocking activity to ROBO2; and a light chain comprising CDR-L1 comprising amino acid sequence of SEQ ID NO: 4 or 7; CDR-L2 comprising amino acid sequence of SEQ ID NO: 5; and CDR-L3 comprising amino acid sequence of SEQ ID NO: 6 and having a specific binding activity to human SLIT2 and/or blocking activity to ROBO2.

In certain frequent embodiments, an antibody is provided that capable of modulating ROBO2 and SLIT2 interaction, wherein the antibody comprises: a heavy chain variable domain comprising an amino acid sequence at least 85% identical to the amino acid sequence of any of SEQ ID NOS: 8, 24-27, optionally wherein the heavy chain variable domain includes CDR-H1, CDR-H2 and CDR-H3 defined by SEQ ID NOS:1-3, respectively, and having a specific binding activity to human SLIT2 and/or blocking activity to ROBO2; and a light chain variable domain comprising an amino acid sequence at least 85% identical to the amino acid sequence of any of SEQ ID NOS: 9, 28-29, optionally wherein the light chain variable domain includes CDR-L1, CDR-L2 and CDR-L3 defined by SEQ ID NOS:4-6, respectively and having a specific binding activity to human SLIT2 and/or blocking activity to ROBO2, or b) a light chain variable domain comprising an amino acid sequence at least 85% identical to any of the amino acid sequence of SEQ ID NOS: 30-31, optionally wherein the light chain variable domain includes CDR-L1, CDR-L2 and CDR-L3 defined by SEQ ID NOS:7, 5 and 6, respectively and having a specific binding activity to human SLIT2 and/or blocking activity to ROBO2.

In certain frequent embodiments, an antibody is provided that binds specifically to human SLIT2 to inhibit binding of human SLIT2 with ROBO2, the antibody comprising: a heavy chain comprising an amino acid sequence at least 90% identical or at least 95% identical to the amino acid sequence of any of SEQ ID NOS: 12, 44-47 and having a specific binding activity to human SLIT2 and/or blocking activity to ROBO2, wherein the heavy chain includes CDR-H1, CDR-H2 and CDR-H3 defined by SEQ ID NOS:1-3, respectively; and a light chain comprising an amino acid sequence at least 90% identical or at least 95% identical to the amino acid sequence of any of SEQ ID NOS: 13, 48-49 and having a specific binding activity to human SLIT2 and/or blocking activity to ROBO2, optionally wherein the light chain variable domain includes CDR-L1, CDR-L2 and CDR-L3 defined by SEQ ID NOS:4-6, respectively, or b) a light chain comprising an amino acid sequence at least 90% identical or at least 95% identical to any of the amino acid sequences of SEQ ID NOS: 50-51 and having a specific binding activity to human SLIT2 and/or blocking activity to ROBO2, optionally wherein the light chain variable domain includes CDR-L1, CDR-L2 and CDR-L3 defined by SEQ ID NOS:7, 5 and 6, respectively.

In frequent embodiments the antibody is a human, chimeric or humanized antibody.

Also in frequently included embodiments, the antibody comprises a human light chain constant region and a human heavy chain constant region. Often, when included, the human light chain constant region is of the IgG1 kappa isotype.

In often preferred embodiments the antibody comprises: a heavy chain variable domain comprising an amino acid sequence at least 85% identical to the amino acid sequence of any of SEQ ID NOS: 8, 24-27, optionally wherein the heavy chain variable domain includes CDR-H1, CDR-H2 and CDR-H3 defined by SEQ ID NOS:1-3, respectively; and/or a light chain variable domain comprising an amino acid sequence at least 85% identical to the amino acid sequence of any of SEQ ID NOS: 9, 28-29, optionally wherein the light chain variable domain includes CDR-L1, CDR-L2 and CDR-L3 defined by SEQ ID NOS:4-6, respectively and having a specific binding activity to human SLIT2 and/or blocking activity to ROBO2, or b) a light chain variable domain comprising an amino acid sequence at least 85% identical to any of the amino acid sequence of SEQ ID NOS: 30-31, optionally wherein the light chain variable domain includes CDR-L1, CDR-L2 and CDR-L3 defined by SEQ ID NOS:7, 5 and 6, respectively. Also often, the antibody comprises: a heavy chain variable domain comprising an amino acid sequence at least 90% identical to the amino acid sequence of any of SEQ ID NOS:8, 24-27, optionally wherein the heavy chain variable domain includes CDR-H1, CDR-H2 and CDR-H3 defined by SEQ ID NOS:1-3, respectively; and/or a light chain variable domain comprising an amino acid sequence at least 90% identical to the amino acid sequence of any of SEQ ID NOS: 9, 28-29, optionally wherein the light chain variable domain includes CDR-L1, CDR-L2 and CDR-L3 defined by SEQ ID NOS:4-6, respectively and having a specific binding activity to human SLIT2 and/or blocking activity to ROBO2, or b) a light chain variable domain comprising an amino acid sequence at least 90% identical to any of the amino acid sequence of SEQ ID NOS: 30-31, optionally wherein the light chain variable domain includes CDR-L1, CDR-L2 and CDR-L3 defined by SEQ ID NOS:7, 5 and 6, respectively. Also often, the antibody comprises: a heavy chain variable domain comprising an amino acid sequence at least 95% identical to the amino acid sequence of any of SEQ ID NOS: 8, 24-27, optionally wherein the heavy chain variable domain includes CDR-H1, CDR-H2 and CDR-H3 defined by SEQ ID NOS:1-3, respectively; and/or a light chain variable domain comprising an amino acid sequence at least 95% identical to the amino acid sequence of any of SEQ ID NOS: 9, 28-29, optionally wherein the light chain variable domain includes CDR-L1, CDR-L2 and CDR-L3 defined by SEQ ID NOS:4-6, respectively and having a specific binding activity to human SLIT2 and/or blocking activity to ROBO2, or b) a light chain variable domain comprising an amino acid sequence at least 95% identical to any of the amino acid sequence of SEQ ID NOS: 30-31, optionally wherein the light chain variable domain includes CDR-L1, CDR-L2 and CDR-L3 defined by SEQ ID NOS:7, 5 and 6, respectively.

In frequently preferred embodiments, a recombinant antibody capable of modulating ROBO2 and SLIT2 interaction is provided, wherein the antibody comprises: a heavy chain comprising an amino acid sequence at least 90% identical to any of the amino acid sequences of SEQ ID NOS: 12, 44-47 and having a specific binding activity to human SLIT2 and/or blocking activity to ROBO2, optionally wherein the heavy chain includes CDR-H1, CDR-H2 and CDR-H3 defined by SEQ ID NOS:1-3, respectively; and a light chain comprising an amino acid sequence at least 90% identical to any of the amino acid sequences of SEQ ID NO: 13, 48-49 and having a specific binding activity to human SLIT2 and/or blocking activity to ROBO2, optionally wherein the light chain variable domain includes CDR-L1, CDR-L2 and CDR-L3 defined by SEQ ID NOS:4-6, respectively, or b) a light chain comprising an amino acid sequence at least 90% identical to any of the amino acid sequences of SEQ ID NOS: 50-51 and having a specific binding activity to human SLIT2 and/or blocking activity to ROBO2, optionally wherein the light chain variable domain includes CDR-L1, CDR-L2 and CDR-L3 defined by SEQ ID NOS:7, 5 and 6, respectively.

Also in frequently preferred embodiments, a recombinant antibody capable of modulating ROBO2 and SLIT2 interaction is provided, wherein the antibody comprises: a heavy chain comprising an amino acid sequence at least 95% identical to any of the amino acid sequences of SEQ ID NOS: 12, 44-47 and having a specific binding activity to human SLIT2 and/or blocking activity to ROBO2, optionally wherein the heavy chain includes CDR-H1, CDR-H2 and CDR-H3 defined by SEQ ID NOS:1-3, respectively; and a light chain comprising an amino acid sequence at least 95% identical to any of the amino acid sequences of SEQ ID NO: 13, 48-49 and having a specific binding activity to human SLIT2 and/or blocking activity to ROBO2, optionally wherein the light chain variable domain includes CDR-L1, CDR-L2 and CDR-L3 defined by SEQ ID NOS:4-6, respectively, or b) a light chain comprising an amino acid sequence at least 95% identical to any of the amino acid sequences of SEQ ID NOS: 50-51 and having a specific binding activity to human SLIT2 and/or blocking activity to ROBO2, optionally wherein the light chain variable domain includes CDR-L1, CDR-L2 and CDR-L3 defined by SEQ ID NOS:7, 5 and 6, respectively.

Also in frequently preferred embodiments, a recombinant antibody capable of modulating ROBO2 and SLIT2 interaction is provided, wherein the antibody comprises: a heavy chain comprising the amino acid sequence of any of the amino acid sequences of SEQ ID NOS: 12, 44-47; and a light chain comprising the amino acid sequence of any of the amino acid sequences of SEQ ID NO: 13, 48-51.

Also in frequently preferred embodiments, an isolated nucleic acid molecule encoding variable regions of an antibody is/are provided, comprising: a nucleotide sequence encoding a heavy chain variable region of SEQ ID NOS: 10, 32-35; and a nucleotide sequence encoding a light chain variable region of SEQ ID NOS: 11, 36-39.

Also in frequently preferred embodiments, an isolated nucleic acid molecule encoding antibody heavy and light chains is/are provided, comprising: a nucleotide sequence encoding a heavy chain of SEQ ID NOS: 14, 52-55; and a nucleotide sequence encoding a light chain of SEQ ID NOS: 15, 56-59.

Also in frequently preferred embodiments, a recombinant antibody heavy chain or fragment thereof having specific binding activity to human SLIT2 and/or blocking activity to ROBO2 is provided, wherein the recombinant antibody heavy chain or fragment thereof comprises the amino acid sequence of SEQ ID NO: 1, 2 and/or 3.

Also in frequently preferred embodiments, a recombinant antibody light chain or fragment thereof having specific binding activity to human SLIT2 and/or blocking activity to ROBO2 is provided, wherein the recombinant antibody light chain or fragment thereof comprises the amino acid sequence of SEQ ID NO: 4, 5, 6, and/or 7.

Also in frequently preferred embodiments, a recombinant antibody heavy chain variable region having specific binding activity to human SLIT2 and/or blocking activity to ROBO2 is provided, wherein the recombinant antibody heavy chain variable region comprises the amino acid sequence of any one of SEQ ID NOS: 8, 24-27.

Also in frequently preferred embodiments, a recombinant antibody light chain variable region having specific binding activity to human SLIT2 and/or blocking activity to ROBO2 is provided, wherein the recombinant antibody light chain variable region comprises the amino acid sequence of any one of SEQ ID NOS: 9, 28-31.

Also in frequently preferred embodiments, a recombinant antibody heavy chain having specific binding activity to human SLIT2 and/or blocking activity to ROBO2 is provided, wherein the recombinant antibody heavy chain comprises the amino acid sequence of any one of SEQ ID NOS: 12, 44-47.

Also in frequently preferred embodiments, a recombinant antibody light chain having specific binding activity to human SLIT2 and/or blocking activity to ROBO2 is provided, wherein the recombinant antibody light chain comprises the amino acid sequence of any one of SEQ ID NOS: 13, 48-51.

Also in frequently preferred embodiments, an antibody is provided comprising a heavy chain variable domain comprising the amino acid sequence of SEQ ID NO: 25; and a light chain variable domain comprising the amino acid sequence of SEQ ID NO: 28.

Also in frequently preferred embodiments, an antibody is provided comprising a heavy chain variable domain comprising the amino acid sequence of SEQ ID NO: 26; and a light chain variable domain comprising the amino acid sequence of SEQ ID NO:30.

Also in frequently preferred embodiments, an antibody is provided comprising a heavy chain comprising the amino acid sequence of SEQ ID NO: 45; and a light chain comprising the amino acid sequence of SEQ ID NO: 48.

Also in frequently preferred embodiments, an antibody is provided comprising a heavy chain comprising the amino acid sequence of SEQ ID NO: 46; and a light chain comprising the amino acid sequence of SEQ ID NO: 50.

Often, the antibody provided comprising an Fc domain, wherein the Fc domain is the Fc domain of an IgA1 IgA2, IgD, IgE, IgM, IgG1, IgG2, IgG3, or IgG4.

In certain embodiments, an antibody is provided that competes for binding to SLIT2 with the antibody, fragment or derivative described above and herein.

Also provided in frequent embodiments are pharmaceutical compositions comprising the anti-SLIT2 antibodies, fragments or derivatives described herein and a pharmaceutically acceptable vehicle.

According to other frequent embodiments, an isolated nucleic acid molecule is provided, comprising one or more nucleotide sequences encoding the anti-SLIT2 antibodies, fragments or derivatives described above and herein. In certain related embodiments, a vector comprising such nucleic acid molecule is provided. Also, in certain related embodiments, a host cell comprising such nucleic acid molecule and/or vector is/are provided.

According to frequent embodiments, methods of making the antibodies described above and herein are provided using the methods and procedures contemplated and described herein, such methods include generating polyclonal antibodies in a host utilizing a SLIT2 protein such as that of SEQ ID NO: 16 or an antigenic fragment thereof, and generating a hybridoma, transfectoma, or other clone to produce such monoclonal antibodies. Such methods of making the also often comprise expressing an anti-SLIT2 antibody, or fragment or derivative thereof described herein, in a host cell and isolating the antibody from the host cell.

Therapeutic methods are also provided here. For example, according to certain embodiments a method of treating a renal disease is provided, comprising administering to a subject in need thereof a therapeutically effective amount of the anti-SLIT2 antibody, fragment or derivative described above and herein, or a pharmaceutical composition comprising such antibody. Often, the renal disease is a glomerular disease, Focal Segmental Glomerular Sclerosis (FSGS), or nephropathy.

Also provided herein are methods of reducing proteinuria, comprising administering to a subject in need thereof a therapeutically effective amount of the anti-SLIT2 antibody, fragment or derivative described above and herein, or a pharmaceutical composition comprising such antibody. Often in such methods, the subject is afflicted with or susceptible to a renal disease, wherein said renal disease is a glomerular disease, Focal Segmental Glomerular Sclerosis (FSGS), or nephropathy.

Also provided herein are methods of preserving podocyte function, comprising administering to a subject in need thereof a therapeutically effective amount of the anti-SLIT2 antibody, fragment or derivative described above and herein, or a pharmaceutical composition comprising such antibody. Often in such methods, the subject is afflicted with or susceptible to a renal disease, wherein said renal disease is a glomerular disease, Focal Segmental Glomerular Sclerosis (FSGS), or nephropathy.

These and other embodiments, features, and advantages will become apparent to those skilled in the art when taken with reference to the following more detailed description of various exemplary embodiments of the present disclosure in conjunction with the accompanying drawings.

### BRIEF DESCRIPTION OF THE DRAWINGS

The skilled person in the art will understand that the drawings, described below, are for illustration purposes only.

FIGS. 1A, 1B, and 1C provide binding data of anti-SLIT2 antibodies described herein with human Slit2, human Slit1 and human Slit 3, respectively, at the cell surface.

FIGS. 2A, 2B and 2C provide binding data of anti-SLIT2 antibodies described herein with human SLit2, cyno Slit2 and rat Slit2, respectively, at the cell surface.

FIGS. 3A and 3B provide blocking data of anti-SLIT2 antibodies described herein to Robo2-Slit2 interaction, at protein level or cellular level, respectively.

FIGS. 4A and 4B provide blocking data of anti-SLIT2 antibodies described herein at cell migration assay.

FIGS. 5A and 5B provide treatment effect of anti-SLIT2 antibodies described herein in reducing the urine albumin-creatine ratio (UACR) and urine albumin-protein ratio (UPCR) in rat Passive Heymann Nephritis (PHN) model.

FIG. 6 provides treatment effect of anti-SLIT2 antibodies described herein in reducing foot process width in rat Passive Heymann Nephritis (PHN) model.

### DETAILED DESCRIPTION

For clarity of disclosure, and not by way of limitation, the detailed description of the invention is divided into the subsections that follow.

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

The antibodies, binding fragments, derivatives, polypeptides and polynucleotides described herein are, in many embodiments, described by way of their respective polypeptide or polynucleotide sequences. Unless indicated otherwise, polypeptide sequences are provided in N→C orientation; polynucleotide sequences in 5′→3′ orientation. For polypeptide sequences, the conventional three or one-letter abbreviations for the genetically encoded amino acids may be used, as noted in TABLE 1, below.

TABLE 1

| Amino Acid | Three Letter Abbreviation | One Letter Abbreviation |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Certain sequences are defined by structural formulae specifying amino acid residues belonging to certain classes (e.g., aliphatic, hydrophobic, etc.). The various classes to which the genetically encoded amino acids belong as used herein are noted in TABLE 2, below. Some amino acids may belong to more than one class. Cysteine, which contains a sulfhydryl group, and proline, which is conformationally constrained, are not assigned classes.

TABLE 2
Encoded Amino Acid Classes

| Class | Amino Acids |
|---|---|
| Aliphatic | A, I, L, V |
| Aromatic | F, Y, W |
| Non-Polar | M, A, I, L, V |
| Polar | N, Q, S, T |
| Basic | H, K, R |
| Acidic | D, E |
| Small | A, G |

As used herein, “a” or “an” means “at least one” or “one or more.”

As used herein, the term “and/or” may mean “and,” it may mean “or,” it may mean “exclusive-or,” it may mean “one,” it may mean “some, but not all,” it may mean “neither,” and/or it may mean “both.”

As used herein, “antibody” refers to polypeptides having an antibody-derived antigen binding site (e.g., a VH/VL region or Fv, or CDR). Such antibodies include known forms of antibodies. For example, the antibody can be a human antibody, a humanized antibody, a bispecific antibody, or a chimeric antibody. The antibody also can be a Fab, Fab′2, ScFv, SMIP, antibody mimetic, nanobody, and/or a domain antibody. The antibody also can be of any of the following isotypes: IgG1, IgG2, IgG3, IgG4, IgM, IgA1, IgA2, IgA sec, IgD, and/or IgE. The antibody may be a naturally occurring antibody or may be an antibody that has been altered (e.g., by mutation, deletion, substitution, conjugation to a non-antibody moiety). For example, an antibody may include one or more variant amino acids (compared to a naturally occurring antibody) that changes a property (e.g., a functional property) of the antibody. For example, numerous such alterations are known in the art which affect, e.g., half-life, effector function, and/or immune responses to the antibody in a patient. The term antibody also includes artificial polypeptide constructs that have at least one antibody-derived antigen binding site.

“Antibody fragment” refers to a molecule that contains the antigen-binding portion of a complete full-length antibody that binds to the antigen that is bound to the complete antibody. Examples of antibody fragments include, but are not limited to, Fv, Fab, Fab′, Fab′-SH, F(ab′)2, diabodies, linear antibodies, single-chain antibody molecules (e.g., scFv), and multispecific antibody fragments. Antibodies, single domain antibodies, VHH nanobodies, domain antibodies, bivalent domain antibodies, or any other fragments of antibodies that bind to an antigen. “VHH” refers to a single domain antibody isolated from camelid animals. In certain embodiments, the VHH comprises the heavy chain variable region of a camel heavy chain antibody. In certain embodiments, the size of VHH does not exceed 25 kDa. In certain embodiments, the size of VHH does not exceed 20 kDa. In certain embodiments, the size of VHH does not exceed 15 kDa.

“Full-length antibody” refers to an antibody comprising two heavy chains and two light chains. The variable regions of the light and heavy chains are responsible for antigen binding. The variable regions of the heavy chain and light chain may be referred to as “VH” and “VL”, respectively. The variable regions in the two chains usually contain three highly variable loops, called complementarity determining regions (CDR) (including LC-CDR1, LC-CDR2 and LC-CDR3 light chain (LC) CDR, including HC-CDR1, HC-CDR2 and HC-CDR3 heavy chain (HC) CDR). The CDR boundaries of the antibodies, binding fragments, and/or derivatives, fragments disclosed herein can be defined or identified by well-known conventions, for example, the conventions of Kabat, Chothia or Al-Lazikani (Al-Lazikani 1997; Chothia 1985; Chothia 1987; Chothia 1989; Kabat 1987; Kabat 1991). In the specific examples and sequences of the present specification, Chothia conventions and numbering are utilized. The three CDRs of the heavy or light chain are inserted between flanking sections called framework regions (FR), which are more conservative than the CDRs and form a scaffold that supports the hypervariable loop. The constant regions of the heavy and light chains do not participate in antigen binding but exhibit multiple effector functions. Antibodies are classified based on the amino acid sequence of the constant region of the antibody heavy chain. The five main classes or isotypes of antibodies are IgA, IgD, IgE, IgG, and IgM, which are characterized by the presence of alpha, delta, epsilon, gamma, and mu heavy chains, respectively. Several major antibody categories are divided into subclasses, such as IgG1 (γ1 heavy chain), IgG2 (γ2 heavy chain), IgG3 (γ3 heavy chain), IgG4 (γ4 heavy chain), IgA1 (α1 heavy chain) or IgA2 (α2 heavy chain) chain).

An antibody that “cross-competes for binding” with a reference antibody refers to an antibody that blocks the binding of the reference antibody to its antigen by more than 50% in a competition assay. On the contrary, in a competition assay, the reference antibody blocks the binding of the antibody to its antigen by more than 50%. Exemplary competitive assays are described in Antibodies, Harlow and Lane (Cold Spring Harbor Press, Cold Spring Harbor, NY).

“Fv” is the smallest antibody fragment, which contains a complete antigen recognition site and an antigen binding site. The fragment consists of a dimer of a heavy chain variable region and a light chain variable region that are tightly non-covalently associated. From the folding of these two domains, six hypervariable loops (3 loops in each of the heavy chain and light chain) are emitted, which contribute amino acid residues for antigen binding and endow the antibody with antigen binding specificity. However, even a single variable domain (or half an Fv containing only three CDRs against the original specificity) can recognize and bind antigen, although sometimes with lower affinity than the complete binding site.

“Single-chain an Fv” (also abbreviated as “sFv” or “the scFv”) is an antibody fragment and V L, V H antibody domains connected into a single polypeptide chain comprising. In some embodiments, the scFv polypeptide further comprises a polypeptide linker between the V H and V L, domain, the polypeptide linker which enables the scFv to form the desired structure for antigen binding. For a review of scFv, see Pluckthun's The Pharmacology of Monoclonal Antibodies, Volume 113, Rosenburg and Moore ed. Springer-Verlag [Springer Press], New York, pp. 269-315 (1994).

For the purposes herein, “acceptor human framework” or “human framework” is a light chain variable domain (VL) framework or heavy chain variable domain (VH) that is derived from a human immunoglobulin framework or a human consensus framework. The framework of the amino acid sequence of the framework. An acceptor human framework “derived from” a human immunoglobulin framework or a human consensus framework may include the same amino acid sequence thereof or may include amino acid sequence changes. In certain embodiments, the number of amino acid changes is 10 or less, 9 or less, 8 or less, 7 or less, 6 or less, 5 or less, 4 or less, 3 or less, or 2 or less. In certain embodiments, the VL acceptor human framework and the VL human immunoglobulin framework sequence or the human consensus framework sequence are identical in sequence.

“Affinity” refers to the strength of the sum of non-covalent interactions between a single binding site of a molecule (e.g., an antibody) and its binding partner (e.g., an antigen). Unless otherwise indicated, as used herein, “binding affinity” refers to internal binding affinity, which reflects a 1:1 interaction between members of a binding pair (e.g., antibody and antigen). The affinity of a molecule X to its partner Y can usually be represented by the dissociation constant (KD). Affinity can be measured by conventional methods known in the art, including those described herein. Specific illustrative and exemplary embodiments for measuring binding affinity are described below.

An “affinity mature” antibody refers to an antibody that has one or more changes in one or more CDRs or hypervariable regions (HVR) compared to a parent antibody that does not have such changes, and the changes provide the antibody to the antigen the improved affinity.

The term “Fc region” or “fragment crystallizable region” as used herein is used to define the C-terminal region of immunoglobulin heavy chains, including native sequence Fc regions and variant Fc regions. Although the boundaries of the Fc region of an immunoglobulin heavy chain can vary, the Fc region of a human IgG heavy chain is generally defined as an amino acid residue at position Cys226 or extending from Pro230 to its carboxyl terminus. The C-terminal lysine (residue 447 according to the EU numbering system) of the Fc region can be removed, for example, during the production or purification of the antibody or by recombinant engineering of the nucleic acid encoding the antibody heavy chain. Therefore, the composition of intact antibodies may include an antibody population with all K447 residues removed, an antibody population without K447 residues removed, and an antibody population with a mixture of antibodies with and without K447 residues. Suitable native sequence Fc regions for the antibodies described herein include human IgG1, IgG2 (IgG2A, IgG2B), IgG3, and IgG4.

“Fc receptor” or “FcR” describes a receptor that binds to the Fc region of an antibody. A preferred FcR is a natural human FcR. In addition, a preferred FcR is an FcR that binds IgG antibodies (γ receptors) and includes receptors of the FcγRI, FcγRII, and FcγRIII subclasses, including allelic variants and spliced forms of these receptors. FcγRII receptors include FcγRIIA (“Activating receptor”) and FcγRIIB (“inhibiting receptor”), they have similar amino acid sequences, the main difference lies in their cytoplasmic domains. The activating receptor FcγRIIA contains an immunoreceptor tyrosine-based activation motif (ITAM) in its cytoplasmic domain. The inhibitory receptor FcγRIIB contains an immunoreceptor tyrosine-based inhibitory motif (ITIM) in its cytoplasmic domain. The term “FcR” herein encompasses other FcRs, including FcRs identified in the future.

As used herein, the term “epitope” refers to a specific atom or amino acid group on the antigen to which the antibody or antigen-binding portion binds. If two antibodies or antigen-binding portions have competitive binding to the antigen, they can bind to the same epitope within the antigen.

As used herein, the terms “specific binding,” “specific binding activity,” “specifically bind,” “specific recognition” and “specific to” refer to a measurable and reproducible interaction, such as the binding between a target and an antibody or antibody portion, which determines the existence of the target in the presence of a population of heterogeneous molecules (including biomolecules). For example, an antibody or antibody portion that specifically recognizes a target (which may be an epitope) is an antibody or antibody portion that binds to the target, and its affinity, avidity, readiness, and/or duration are longer than binding to other targets. In some embodiments, the degree of binding of the antibody to the unrelated target is less than about 10% of the degree of binding of the antibody to the target as measured by radioimmunoassay (RIA), for example. In some embodiments, the dissociation constant (KD) of the antibody that specifically binds to the target is ≤10-5 M, ≤10-6 M, ≤10-7 M, ≤10-8 M, ≤10-9 M, ≤510-10 M, ≤10-11 M, or ≤10-12 M. In some embodiments, the antibody specifically binds to an epitope of a protein that is conserved from proteins of different species. In some embodiments, specific binding may include but does not require exclusive binding. The binding specificity of an antibody or antigen-binding domain can be determined experimentally by methods known in the art. Such methods include, but are not limited to, Western blot, ELISA-, RIA-, ECL-, IRMA-, EIA-, BIACORE™-test, and peptide scanning.

An “isolated” antibody (or construct) is an antibody that has been identified, separated, and/or recovered from a component (e.g., natural or recombinant) of its production environment. In certain embodiments, the isolated polypeptide has no or substantially no association with all other components in its production environment.

An “isolated” nucleic acid molecule encoding a construct, antibody, antigen-binding fragment or derivative thereof described herein is a nucleic acid molecule that has been identified and separated from at least one contaminant nucleic acid molecule that is normally associated with it in its production environment. In certain embodiments, the isolated nucleic acid has no or substantially no association with all components related to the production environment. The form of the isolated nucleic acid molecules encoding the polypeptides and antibodies described herein is different from the naturally occurring form or background. Therefore, the isolated nucleic acid molecule is different from the nucleic acid encoding the polypeptides and antibodies described herein that are naturally present in the cell. An isolated nucleic acid includes the nucleic acid molecule contained in a cell that usually contains the nucleic acid molecule, but the nucleic acid molecule exists outside the chromosome or at a chromosomal location different from its natural chromosomal location.

The term “nucleic acid” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues.

The term “nucleotide sequence” refers to a polymer of DNA or RNA that can be single- or double-stranded, optionally containing synthetic, non-natural or altered nucleotide bases capable of incorporation into DNA or RNA polymers.

The terms “nucleic acid”, “nucleic acid molecule”, “nucleic acid fragment”, “nucleic acid sequence or segment”, or “polynucleotide” are used interchangeably and may also be used interchangeably with gene, cDNA, DNA and RNA encoded by a gene.

The term “chimeric” refers to a gene or DNA that contains 1) DNA sequences, including regulatory and coding sequences, that are not found together in nature, or 2) sequences encoding parts of proteins not naturally adjoined, or 3) parts of promoters that are not naturally adjoined. Accordingly, a chimeric gene may include regulatory sequences and coding sequences that are derived from different sources or include regulatory sequences and coding sequences derived from the same source, but are arranged in a manner different from that found in nature.

The term “chimeric” antibody as used herein refers to an antibody having variable sequences derived from a non-human immunoglobulin, such as a rat or a mouse antibody, and human immunoglobulin constant regions, typically chosen from a human immunoglobulin template. The antibodies or their fragments of chimeric type according to the present disclosure can be prepared by using the techniques of genetic recombination. For example, the chimeric antibody can be produced by cloning a recombinant DNA containing a promoter and a sequence coding for the variable region of a non-human, especially murine, monoclonal antibody according to the invention and a sequence coding for the constant region of human antibody. A chimeric antibody of the invention encoded by such a recombinant gene will be, for example, a mouse-man chimera, the specificity of this antibody being determined by the variable region derived from the murine DNA and its isotype determined by the constant region derived from the human DNA. These and other methods for producing chimeric antibodies are known in the art. See, e.g., Morrison, 1985, Science 229(4719):1202-7; Oi et al., 1986, BioTechniques 4:214-221; Gillies et al., 1985, J. Immunol. Methods 125:191-202; U.S. Pat. Nos. 5,807,715; 4,816,567; and 4,816,397, which are incorporated herein by reference in their entireties.

“Conservatively modified variations” of a particular nucleic acid sequence refers to those nucleic acid sequences that encode identical or essentially identical amino acid sequences. Because of the degeneracy of the genetic code, a large number of functionally identical nucleic acids encode any given polypeptide. For instance, the codons CGT, CGC, CGA, CGG, AGA and AGG all encode the amino acid arginine. Thus, at every position where an arginine is specified by a codon, the codon can be altered to any of the corresponding codons described without altering the encoded protein. Such nucleic acid variations are “silent variations,” which are one species of “conservatively modified variations.” Every nucleic acid sequence described herein that encodes a polypeptide also describes every possible silent variation, except where otherwise noted. One of skills in the art will recognize that each codon in a nucleic acid (except ATG, which is ordinarily the only codon for methionine) can be modified to yield a functionally identical molecule by standard techniques. Accordingly, each “silent variation” of a nucleic acid that encodes a polypeptide is implicit in each described sequence.

“Coding sequence” refers to a DNA or RNA sequence that codes for a specific amino acid sequence. It may constitute an “uninterrupted coding sequence”, i.e., lacking an intron, such as in a cDNA, or it may include one or more introns bounded by appropriate splice junctions. An “intron” is a sequence of RNA that is contained in the primary transcript but is removed through cleavage and re-ligation of the RNA within the cell to create the mature mRNA that can be translated into a protein.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of molecular biology techniques, as, for example, with polymerase chain reaction (PCR) and hybridization techniques. Variant nucleotide sequences also include synthetically derived nucleotide sequences, such as those generated, for example, by using site-directed mutagenesis, which encode the native protein, as well as those that encode a polypeptide having amino acid substitutions. Generally, nucleotide sequence variants of the invention will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

The term “control sequence” refers to a DNA sequence necessary for the expression of an operably linked coding sequence in a specific host organism. For example, suitable control sequences for prokaryotes include promoters, optional operator sequences, and ribosome binding sites. It is known that eukaryotic cells utilize promoters, polyadenylation signals and enhancers.

A nucleic acid is “operably linked” when it is in a functional relationship with another nucleic acid sequence. For example, if the DNA of the pre-sequence or the secretory leader sequence is expressed as a pre-protein involved in the secretion of the polypeptide, the DNA of the pre-sequence or the secretory leader sequence is operably linked to the DNA of the polypeptide; if the promoter or enhancer affects for transcription of the coding sequence, the promoter or enhancer is operably linked to the sequence; or if the ribosome binding site is positioned so as to facilitate translation, the ribosome binding side is operably linked to the coding sequence. Generally, “operably linked” means that the linked DNA sequences are continuous and, in the case of a secreted leader sequence, are continuous and in reading frame. However, the enhancer need not be continuous. The connection is achieved by connecting at convenient restriction sites. If such sites are not present, synthetic oligonucleotide adaptors or linkers are used according to conventional practice.

As used herein, the term “vector” refers to a nucleic acid molecule capable of propagating another nucleic acid linked to it. The term includes vectors that are self-replicating nucleic acid structures, as well as vectors that have been incorporated into the genome of a host cell into which they have been introduced. Certain vectors can direct the expression of nucleic acids to which they are operably linked. Such vectors are referred to herein as “expression vectors”.

As used herein, the term “transfected” or “transformed” or “transduced” refers to the process of transferring or introducing exogenous nucleic acid into a host cell. A “transfected” or “transformed” or “transduced” cell is a cell that has been transfected, transformed or transduced with an exogenous nucleic acid, and the cell includes the primary target cell and its progeny.

The terms “host cell,” “host cell line” and “host cell culture” are used interchangeably and refer to cells into which exogenous nucleic acid has been introduced, including the progeny of such cells. Host cells include “transformants” and “transformed cells”, which include primary transformed cells and progeny derived therefrom, regardless of the number of passages. The nucleic acid content of the offspring may not be exactly the same as the parent cell, and, may contain mutations. Mutant progeny that have the same function or biological activity as the function or biological activity screened or selected in the original transformed cell are included herein.

The terms “subject,” “individual” and “patient” are used interchangeably herein and refer to mammals, including but not limited to humans, cows, horses, cats, dogs, rodents, or primates. In some embodiments, the subject is a human.

The “effective amount” of an agent refers to an amount effective to achieve the desired therapeutic or preventive result within the necessary dose and time period. The specific dose can be changed according to one or more of the following: the specific agent selected, the subsequent dosing regimen (regardless of whether it is combined with other compounds), the time of administration, the tissue that is imaged, and where it is carried physical delivery system.

The “therapeutically effective amount” of the substance/molecule, agonist or antagonist of the present application can be based on, for example, the disease state, age, sex, and individual weight, and the ability of the substance/molecule, agonist or antagonist to elicit a desired response in the individual and other factors. The therapeutically effective amount is also the amount at which any toxic or harmful effects of the substance/molecule, agonist or antagonist are offset by the beneficial effects of the treatment. The therapeutically effective amount can be delivered by one or more administrations.

“Prophylactically effective amount” refers to an effective amount in a dose meter and for a required period of time to achieve the desired preventive result. Typically, but not necessarily, because the preventive dose is used in the subject before or early in the disease, the preventive effective amount will be less than the therapeutically effective amount.

As used herein, “treatment or treating” is a method used to obtain beneficial or desired results (including clinical results). For the purposes of this application, beneficial or desired clinical results include, but are not limited to, one or more of the following: alleviation of one or more symptoms caused by the disease, reduction of the degree of the disease, stabilization of the disease (for example, prevent or delay the deterioration of the disease), prevent or delay the spread of the disease (for example, metastasis), prevent or delay the recurrence of the disease, delay or slow the progression of the disease, improve the disease state, provide relief (partial or full), and reduce the dosage of one or more other drugs required to treat the disease, delay the progression of the disease, increase or improve the quality of life, increase weight gain and/or prolong survival. “Treatment” also encompasses reducing the pathological consequences of cancer (like, for example, tumor volume). The method of the application considers any one or more of these therapeutic aspects. “Treatment” does not necessarily mean that the disease being treated will be cured.

It should be understood that the examples of the application described herein include “consisting of” and/or “essentially consisting of.”

As used herein, the term “about” or “approximately” means that a specific value determined by a person of ordinary skill in the art is within an acceptable error range, which will depend in part on how the value is determined or deterministic, that is, limited by the measurement system. In certain embodiments, “about” may mean within 3 or more standard deviations according to practice in the art. In certain embodiments, “about” may mean a range of at most 20% (e.g., at most 10%, at most 5%, or at most 1%) of a given value. In certain embodiments, particularly for biological systems and methods, the term may mean within the order of a certain value, such as within 5 times or within 2 times.

As used herein, the term “modulation” refers to a positive or negative change. Exemplary adjustments include changes of about 1%, about 2%, about 5%, about 10%, about 25%, about 50%, about 75%, or about 100%.

As used herein, the term “increase” refers to a positive change of at least about 5%. The change can be about 5%, about 10%, about 25%, about 30%, about 50%, about 75%, about 100%, or more.

As used herein, the term “decrease” refers to a negative change of at least about 5%. The change can be about 5%, about 10%, about 25%, about 30%, about 50%, about 75%, or even about 100%.

The term “about X-Y” as used herein has the same meaning as “about X to about Y”.

When used herein and in the appended claims, the singular forms “a”, “or” and “the” include plural usages unless the context clearly indicates singular.

Antibodies, binding fragments, and/or derivatives, composing the anti-SLIT2 antibodies generally comprise a heavy chain comprising a variable region (V_{H}) having three complementarity determining regions (“CDRs”) referred to herein (in N→C order) as V_{H} CDR #1, V_{H} CDR #2, and V_{H} CDR #3, and a light chain comprising a variable region (V_{L}) having three complementarity determining regions referred to herein (in N→C order) as V_{L} CDR #1, V_{L} CDR #2, and V_{L} CDR #3. The amino acid sequences of exemplary CDRs, as well as the amino acid sequence of the V_{H} and V_{L} regions of the heavy and light chains of exemplary anti-SLIT2 antibodies, binding fragments, and/or derivatives that can be included in antigen binding moieties are provided herein. Specific embodiments of anti-SLIT2 antibodies include, but are not limited to, those that comprise antibodies, binding fragments, and/or derivatives that include these exemplary CDRs and/or V_{H} and/or V_{L} sequences, as well as antibodies, binding fragments, and/or derivatives that compete for binding SLIT2 with such antibodies, binding fragments, and/or derivatives. Tables 3 and 4 set forth the amino acid and nucleic acid sequences of the heavy and light chain CDRs of exemplary anti-SLIT2 antibodies, including 75C1 and 75C1 substitution mutants. Tables 5-8 set forth heavy and light chain variable region amino acid and nucleic acid sequences of an exemplary anti-SLIT2 antibodies and anti-SLIT2 antibody substitution mutants of the present disclosure.

TABLE 3
Amino acid sequences of the heavy and light
chain CDRs of 75C1 and 75C1 substitution
mutants

| | HCDR1 | HCDR2 | HCDR3 | LCDR1 | LCDR2 | LCDR3 |
|---|---|---|---|---|---|---|
| 75C1 | SEQ ID | SEQ ID | SEQ ID | SEQ ID | SEQ ID | SEQ ID |
| | NO: 1 | NO: 2 | NO: 3 | NO: 4 | NO: 5 | NO: 6 |
| | GGSF | IPIF | GSGWY | RASQNI | AASN | QQSYR |
| | RSY | GT | TFDY | NSYLN | LQS | SPPT |
| 75C1_ | SEQ ID | SEQ ID | SEQ ID | SEQ ID | SEQ ID | SEQ ID |
| LC_ | NO: 1 | NO: 2 | NO: 3 | NO: 7 | NO: 5 | NO: 6 |
| MUT_ | GGSF | IPIF | GSGWY | RASQNI | AASN | QQSYR |
| 2.1, | RSY | GT | TFDY | NAYLN | LQS | SPPT |
| 2.2 | | | | | | |

TABLE 4
Nucleic acid sequences of the heavy and light chain
CDRs of 75C1 and 75C1 substitution mutants

| | HCDR1 | HCDR2 | HCDR3 | LCDR1 | LCDR2 | LCDR3 |
|---|---|---|---|---|---|---|
| 75C1 | SEQ ID | SEQ ID | SEQ ID | SEQ ID | SEQ ID | SEQ ID |
| | NO: 17 | NO: 18 | NO: 19 | NO: 20 | NO: 21 | NO: 22 |
| | GGAGG | ATCCC | GGTAG | CGGGC | GCTGC | CAACA |
| | CTCCT | TATCT | TGGCT | GAGTC | ATCCA | GAGCT |
| | TCAGG | TTGGT | GGTAC | AAAAC | ATTTG | ATAGA |
| | AGCTAT | ACA | ACCTT | ATTAA | CAAAG | AGTCC |
| | | | TGACT | CAGCT | T | TCCCA |
| | | | AC | ATTTA | | CT |
| | | | | AAT | | |
| 75C1_ | SEQ ID | SEQ ID | SEQ ID | SEQ ID | SEQ ID | SEQ ID |
| LC_ | NO: 17 | NO: 18 | NO: 19 | NO: 23 | NO: 21 | NO: 22 |
| MUT_ | GGAGG | ATCCC | GGTAGT | CGGGC | GCTGC | CAACAG |
| 2.1, | CTCCT | TATCT | GGCTGG | GAGTC | ATCCA | AGCTAT |
| 2.2 | TCAGG | TTGGT | TACACC | AAAAC | ATTTG | AGAAGT |
| | AGCTA | ACA | TTTGAC | ATTAA | CAAAG | CCTCCC |
| | T | | TAC | CGCGT | T | ACT |
| | | | | ATTTA | | |
| | | | | AAT | | |

TABLE 5
Amino acid sequences of the variable
regions of 75C1

| | VH | VL |
|---|---|---|
| 75C1 | SEQ ID NO: 8 | SEQ ID NO: 9 |
| | QVQLVQSGAEVKNPGSSVK | DIQMTQSPSSLSASIGDR |
| | VSCKASGGSFRSYTVSWVR | VTITCRASQNINSYLNWY |
| | QAPGQGLEWMGGIIPIFGT | QQKPGKAPKFLIYAASNL |
| | ANYAQKFQGRVTITADEST | QSGVPSRFSGSGSGTDEN |
| | DTTYMDLSSLKSENTAVYY | LNISSLQPEDFAIYYCQQ |
| | CATGSGWYTFDYWGQGTLV | SYRSPPTFGGGTTVEIK |
| | TVSS | |

TABLE 6
Nucleic acid sequences of the variable regions of 75C1

| | VH | VL |
|---|---|---|
| 75C1 | SEQ ID NO: 10 | SEQ ID NO: 11 |
| | CAGGTGCAGCTGGTGCAGTCTGGGGCT | GACATCCAGATGACCCAGTCTCCATCCT |
| | GAGGTGAAGAACCCTGGGTCCTCGGTG | CCCTGTCTGCATCTATAGGAGACAGAGT |
| | AAGGTCTCCTGCAAGGCTTCTGGAGGC | CACCATCACTTGCCGGGCGAGTCAAAAC |
| | TCCTTCAGGAGCTATACTGTCAGTTGG | ATTAACAGCTATTTAAATTGGTATCAGC |
| | GTGCGACAGGCCCCTGGACAAGGGCTT | AGAAACCAGGGAAAGCCCCTAAGTTCCT |
| | GAGTGGATGGGAGGGATCATCCCTATC | GATCTATGCTGCATCCAATTTGCAAAGT |
| | TTTGGTACAGCAAACTACGCACAGAAG | GGGGTCCCATCAAGGTTCAGTGGCAGTG |
| | TTCCAGGGCAGAGTCACGATTACCGCG | GATCTGGGACAGATTTCAATCTCAACAT |
| | GACGAATCCACGGACACAACCTACATG | CAGCAGTCTGCAGCCTGAAGATTTTGCA |
| | GACCTGAGCAGCCTGAAATCTGAAAAC | ATTTACTACTGTCAACAGAGCTATAGAA |
| | ACGGCCGTGTATTATTGTGCGACAGGT | GTCCTCCCACTTTCGGCGGAGGGACCAC |
| | AGTGGCTGGTACACCTTTGACTACTGG | GGTGGAGATTAAA |
| | GGCCAGGGAACCCTGGTCACCGTCTCC | |
| | TCA | |

TABLE 7
Amino acid sequences of the variable regions for substitution mutants

| Name | Variable Region Amino Acid Sequences | Mutations |
|---|---|---|
| 75C1_HC_MUT_1.1 | SEQ ID NO: 24 | N13Q |
| (HEAVY CHAIN) | QVQLVQSGAEVKQPGSSVKVSCKASGGSFRSYTVSWVR | |
| | QAPGQGLEWMGGIIPIFGTANYAQKFQGRVTITADEST | |
| | DTTYMDLSSLKSENTAVYYCATGSGWYTFDYWGQGTLV | |
| | TVSS | |
| 75C1_HC_MUT_1.2 | SEQ ID NO: 25 | N13Q, |
| (HEAVY CHAIN) | QVQLVQSGAEVKQPGSSVKVSCKASGGSFRSYTVSWVR | T78V |
| | QAPGQGLEWMGGIIPIFGTANYAQKFQGRVTITADEST | |
| | DTVYMDLSSLKSENTAVYYCATGSGWYTFDYWGQGTLV | |
| | TVSS | |
| 75C1_HC_MUT_1.3 | SEQ ID NO: 26 | N13Q, |
| (HEAVY CHAIN) | QVQLVQSGAEVKQPGSSVKVSCKASGGSFRSYTVSWVR | T78V, |
| | QAPGQGLEWMGGIIPIFGTANYAQKFQGRVTITADEST | N86D |
| | DTVYMDLSSLKSEDTAVYYCATGSGWYTFDYWGQGTLV | |
| | TVSS | |
| 75C1_HC_MUT_1.4 | SEQ ID NO: 27 | N13Q, |
| (HEAVY CHAIN) | QVQLVQSGAEVKQPGSSVKVSCKASGGSFRSYTVSWVR | N86D |
| | QAPGQGLEWMGGIIPIFGTANYAQKFQGRVTITADEST | |
| | DTTYMDLSSLKSEDTAVYYCATGSGWYTFDYWGQGTLV | |
| | TVSS | |
| 75C1_LC_MUT_1.1 | SEQ ID NO: 28 | N72T, |
| (LIGHT CHAIN) | DIQMTQSPSSLSASIGDRVTITCRASQNINSYLNWYQQ | N74T |
| | KPGKAPKFLIYAASNLQSGVPSRFSGSGSGTDFTLTIS | |
| | SLQPEDFAIYYCQQSYRSPPTFGGGTTVEIK | |
| 75C1_LC_MUT_1.2 | SEQ ID NO: 29 | N72T |
| (LIGHT CHAIN) | DIQMTQSPSSLSASIGDRVTITCRASQNINSYLNWYQQ | |
| | KPGKAPKFLIYAASNLQSGVPSRFSGSGSGTDFTLNIS | |
| | SLQPEDFAIYYCQQSYRSPPTFGGGTTVEIK | |
| 75C1_LC_MUT_2.1 | SEQ ID NO: 30 | N72T, |
| (LIGHT CHAIN) | DIQMTQSPSSLSASIGDRVTITCRASQNINAYLNWYQQ | N74T, S31A |
| | KPGKAPKFLIYAASNLQSGVPSRFSGSGSGTDFTLTIS | |
| | SLQPEDFAIYYCQQSYRSPPTFGGGTTVEIK | |
| 75C1_LC_MUT_2.2 | SEQ ID NO: 31 | N72T, S31A |
| (LIGHT CHAIN) | DIQMTQSPSSLSASIGDRVTITCRASQNINAYLNWYQQ | |
| | KPGKAPKFLIYAASNLQSGVPSRFSGSGSGTDFTLNIS | |
| | SLQPEDFAIYYCQQSYRSPPTFGGGTTVEIK | |

TABLE 8
Nucleic acid sequences of the variable regions for substitution mutants

| Name | Variable Region Nucleic Acid Sequences |
|---|---|
| 75C1_HC_MUT_1.1 | SEQ ID NO: 32 |
| (HEAVY CHAIN) | CAGGTGCAGCTGGTGCAGTCTGGGGCTGAGGTGAAGCA |
| | ACCTGGGTCCTCGGTGAAGGTCTCCTGCAAGGCTTCTG |
| | GAGGCTCCTTCAGGAGCTATACTGTCAGTTGGGTGCGA |
| | CAGGCCCCTGGACAAGGGCTTGAGTGGATGGGAGGGAT |
| | CATCCCTATCTTTGGTACAGCAAACTACGCACAGAAGT |
| | TCCAGGGCAGAGTCACGATTACCGCGGACGAATCCACG |
| | GACACAACCTACATGGACCTGAGCAGCCTGAAATCTGA |
| | AAACACGGCCGTGTATTATTGTGCGACAGGTAGTGGCT |
| | GGTACACCTTTGACTACTGGGGCCAGGGAACCCTGGTC |
| | ACCGTCTCCTCA |
| 75C1_HC_MUT_1.2 | SEQ ID NO: 33 |
| (HEAVY CHAIN) | CAGGTGCAGCTGGTGCAGTCTGGGGCTGAGGTGAAGCA |
| | ACCTGGGTCCTCGGTGAAGGTCTCCTGCAAGGCTTCTG |
| | GAGGCTCCTTCAGGAGCTATACTGTCAGTTGGGTGCGA |
| | CAGGCCCCTGGACAAGGGCTTGAGTGGATGGGAGGGAT |
| | CATCCCTATCTTTGGTACAGCAAACTACGCACAGAAGT |
| | TCCAGGGCAGAGTCACGATTACCGCGGACGAATCCACG |
| | GACACAGTATACATGGACCTGAGCAGCCTGAAATCTGA |
| | AAACACGGCCGTGTATTATTGTGCGACAGGTAGTGGCT |
| | GGTACACCTTTGACTACTGGGGCCAGGGAACCCTGGTC |
| | ACCGTCTCCTCA |
| 75C1_HC_MUT_1.3 | SEQ ID NO: 34 |
| (HEAVY CHAIN) | CAGGTGCAGCTGGTGCAGTCTGGGGCTGAGGTGAAGCA |
| | ACCTGGGTCCTCGGTGAAGGTCTCCTGCAAGGCTTCTG |
| | GAGGCTCCTTCAGGAGCTATACTGTCAGTTGGGTGCGA |
| | CAGGCCCCTGGACAAGGGCTTGAGTGGATGGGAGGGAT |
| | CATCCCTATCTTTGGTACAGCAAACTACGCACAGAAGT |
| | TCCAGGGCAGAGTCACGATTACCGCGGACGAATCCACG |
| | GACACAGTATACATGGACCTGAGCAGCCTGAAATCTGA |
| | AGATACGGCCGTGTATTATTGTGCGACAGGTAGTGGCT |
| | GGTACACCTTTGACTACTGGGGCCAGGGAACCCTGGTC |
| | ACCGTCTCCTCA |
| 75C1_HC_MUT_1.4 | SEQ ID NO: 35 |
| (HEAVY CHAIN) | CAGGTGCAGCTGGTGCAGTCTGGGGCTGAGGTGAAGCA |
| | ACCTGGGTCCTCGGTGAAGGTCTCCTGCAAGGCTTCTG |
| | GAGGCTCCTTCAGGAGCTATACTGTCAGTTGGGTGCGA |
| | CAGGCCCCTGGACAAGGGCTTGAGTGGATGGGAGGGAT |
| | CATCCCTATCTTTGGTACAGCAAACTACGCACAGAAGT |
| | TCCAGGGCAGAGTCACGATTACCGCGGACGAATCCACG |
| | GACACAACCTACATGGACCTGAGCAGCCTGAAATCTGA |
| | AGACACGGCCGTGTATTATTGTGCGACAGGTAGTGGCT |
| | GGTACACCTTTGACTACTGGGGCCAGGGAACCCTGGTC |
| | ACCGTCTCCTCA |
| 75C1_LC_MUT_1.1 | SEQ ID NO: 36 |
| (LIGHT CHAIN) | GACATCCAGATGACCCAGTCTCCATCCTCCCTGTCTGCA |
| | TCTATAGGAGACAGAGTCACCATCACTTGCCGGGCGAGT |
| | CAAAACATTAACAGCTATTTAAATTGGTATCAGCAGAAA |
| | CCAGGGAAAGCCCCTAAGTTCCTGATCTATGCTGCATCC |
| | AATTTGCAAAGTGGGGTCCCATCAAGGTTCAGTGGCAGT |
| | GGATCTGGGACAGATTTCACACTCACTATCAGCAGTCTG |
| | CAGCCTGAAGATTTTGCAATTTACTACTGTCAACAGAGC |
| | TATAGAAGTCCTCCCACTTTCGGCGGAGGGACCACGGTG |
| | GAGATTAAA |
| 75C1_LC_MUT_1.2 | SEQ ID NO: 37 |
| (LIGHT CHAIN) | GACATCCAGATGACCCAGTCTCCATCCTCCCTGTCTGCA |
| | TCTATAGGAGACAGAGTCACCATCACTTGCCGGGCGAGT |
| | CAAAACATTAACAGCTATTTAAATTGGTATCAGCAGAAA |
| | CCAGGGAAAGCCCCTAAGTTCCTGATCTATGCTGCATCC |
| | AATTTGCAAAGTGGGGTCCCATCAAGGTTCAGTGGCAGT |
| | GGATCTGGGACAGATTTCACACTCAACATCAGCAGTCTG |
| | CAGCCTGAAGATTTTGCAATTTACTACTGTCAACAGAGC |
| | TATAGAAGTCCTCCCACTTTCGGCGGAGGGACCACGGTG |
| | GAGATTAAA |
| 75C1_LC_MUT_2.1 | SEQ ID NO: 38 |
| (LIGHT CHAIN) | GACATCCAGATGACCCAGTCTCCATCCTCCCTGTCTGCA |
| | TCTATAGGAGACAGAGTCACCATCACTTGCCGGGCGAGT |
| | CAAAACATTAACGCGTATTTAAATTGGTATCAGCAGAAA |
| | CCAGGGAAAGCCCCTAAGTTCCTGATCTATGCTGCATCC |
| | AATTTGCAAAGTGGGGTCCCATCAAGGTTCAGTGGCAGT |
| | GGATCTGGGACAGATTTCACACTCACTATCAGCAGTCTG |
| | CAGCCTGAAGATTTTGCAATTTACTACTGTCAACAGAGC |
| | TATAGAAGTCCTCCCACTTTCGGCGGAGGGACCACGGTG |
| | GAGATTAAA |
| 75C1_LC_MUT_2.2 | SEQ ID NO: 39 |
| (LIGHT CHAIN) | GACATCCAGATGACCCAGTCTCCATCCTCCCTGTCTGCA |
| | TCTATAGGAGACAGAGTCACCATCACTTGCCGGGCGAGT |
| | CAAAACATTAACGCGTATTTAAATTGGTATCAGCAGAAA |
| | CCAGGGAAAGCCCCTAAGTTCCTGATCTATGCTGCATCC |
| | AATTTGCAAAGTGGGGTCCCATCAAGGTTCAGTGGCAGT |
| | GGATCTGGGACAGATTTCACACTCAACATCAGCAGTCTG |
| | CAGCCTGAAGATTTTGCAATTTACTACTGTCAACAGAGC |
| | TATAGAAGTCCTCCCACTTTCGGCGGAGGGACCACGGTG |
| | GAGATTAAA |

It has been found that the CDRs disclosed in Tables 3 and 4 provide an aspect common to antibodies described herein that render them particularly suitable to specific binding the SLIT2 protein (including antigenic polypeptides thereof) and to specific binding of SLIT2 protein and inhibition and/or prohibition of the SLIT2-ROBO2 interaction (i.e., blocking activity to ROBO2). This includes human SLIT2 in addition to SLIT2 in other species.

It has also been found that the CDRs disclosed in Tables 3 and 4 provide an aspect common to antibodies described herein that render them particularly suitable to specific binding the SLIT2 protein (including antigenic polypeptides thereof) and to specific binding of SLIT2 protein and inhibition and/or prohibition of the SLIT2-ROBO2 interaction (i.e., blocking activity to ROBO2) as part of treating a renal disease, such as a glomerular disease, Focal Segmental Glomerular Sclerosis (FSGS), or nephropathy.

It has also been found that the CDRs disclosed in Tables 3 and 4 provide an aspect common to antibodies described herein that render them particularly suitable to specific binding the SLIT2 protein (including antigenic polypeptides thereof) and to specific binding of SLIT2 protein and inhibition and/or prohibition of the SLIT2-ROBO2 interaction (i.e., blocking activity to ROBO2) as part of reducing proteinuria, including in subjects susceptible to or afflicted with renal disease such as glomerular disease, Focal Segmental Glomerular Sclerosis (FSGS), or nephropathy.

It has also been found that the CDRs disclosed in Tables 3 and 4 provide an aspect common to antibodies described herein that render them particularly suitable to specific binding the SLIT2 protein (including antigenic polypeptides thereof) and to specific binding of SLIT2 protein and inhibition and/or prohibition of the SLIT2-ROBO2 interaction (i.e., blocking activity to ROBO2) as part of preserving podocyte function, including in subjects susceptible to or afflicted with renal disease such as glomerular disease, Focal Segmental Glomerular Sclerosis (FSGS), or nephropathy.

It has also been found that the mutations disclosed in Table 9 provide an aspect common to antibodies where mutations in the described locations and in the manner described (alone or in the identified VH-VL pairings) retain their particular suitability to specifically binding the SLIT2 protein (including antigenic polypeptides thereof) and to specific binding of SLIT2 protein and inhibition and/or prohibition of the SLIT2-ROBO2 interaction (i.e., blocking activity to ROBO2). This includes human SLIT2 in addition to SLIT2 in other species.

It has also been found that the mutations disclosed in Table 9 provide an aspect common to antibodies where mutations in the described locations and in the manner described (alone or in the identified VH-VL pairings) retain their particular suitability to specifically binding of SLIT2 protein and inhibition and/or prohibition of the SLIT2-ROBO2 interaction (i.e., blocking activity to ROBO2) as part of treating a renal disease, such as a glomerular disease, Focal Segmental Glomerular Sclerosis (FSGS), or nephropathy.

It has also been found that the mutations disclosed in Table 9 provide an aspect common to antibodies where mutations in the described locations and in the manner described (alone or in the identified VH-VL pairings) retain their particular suitability to specifically binding the SLIT2 protein (including antigenic polypeptides thereof) and to specific binding of SLIT2 protein and inhibition and/or prohibition of the SLIT2-ROBO2 interaction (i.e., blocking activity to ROBO2) as part of reducing proteinuria, including in subjects susceptible to or afflicted with renal disease such as glomerular disease, Focal Segmental Glomerular Sclerosis (FSGS), or nephropathy.

It has also been found that the mutations disclosed in Table 9 provide an aspect common to antibodies where mutations in the described locations and in the manner described (alone or in the identified VH-VL pairings) retain their particular suitability to specifically binding of SLIT2 protein and inhibition and/or prohibition of the SLIT2-ROBO2 interaction (i.e., blocking activity to ROBO2) as part of preserving podocyte function, including in subjects susceptible to or afflicted with renal disease such as glomerular disease, Focal Segmental Glomerular Sclerosis (FSGS), or nephropathy.

Antibodies may be in the form of full-length antibodies, bispecific antibodies, dual variable domain antibodies, multiple chain or single chain antibodies, surrobodies (including surrogate light chain construct), single domain antibodies, camelized antibodies, scFv-Fc antibodies, and the like. They may be of, or derived from, any isotype, including, for example, IgA (e.g., IgA₁ or IgA₂), IgD, IgE, IgG (e.g., IgG₁, IgG₂, IgG₃ or IgG₄), IgM, or IgY. In some embodiments, the anti-SLIT2 antibody is an IgG (e.g., IgG₁, IgG₂, IgG₃ or IgG₄). Antibodies may be of human or non-human origin. Examples of non-human origin include, but are not limited to, mammalian origin (e.g., simians, rodents, goats, and rabbits) or avian origin (e.g., chickens). In specific embodiments, antibodies composing the anti-SLIT2 antibodies, binding fragments and/or derivatives are suitable for administration to humans, such as, for example, humanized antibodies and/or fully human antibodies.

Anti-SLIT2 antibodies may be polyclonal, monoclonal, genetically engineered, and/or otherwise modified in nature, including, but not limited to, chimeric antibodies, humanized antibodies, human antibodies, single chain antibodies, bispecific antibodies, dual-variable domain antibodies, etc. In various embodiments, the antibodies comprise all or a portion of a constant region of an antibody. In some embodiments, the constant region is an isotype selected from: IgA (e.g., IgA₁ or IgA₂), IgD, IgE, IgG (e.g., IgG₁, IgG₂, IgG₃ or IgG₄), IgM, and IgY. In specific embodiments, the anti-SLIT2 antibody, binding fragment and/or derivative comprises an IgG₁ constant region isotype.

The present disclosure relates to a monoclonal antibody, or a divalent functional fragment or derivative thereof, capable to inhibit the SLIT2-ROBO2 interaction and comprising a heavy chain comprising CDR-H1, CDR-H2 and CDR-H3 with respectively the amino acid sequences SEQ ID Nos. 1, 2, and/or 3 or a sequence having at least 80% identity after optimum alignment with sequences SEQ ID Nos. 1, 2, and/or 3; and a light chain comprising CDR-L1, CDR-L2 and CDR-L3 with respectively the amino acid sequences SEQ ID Nos. 4, 5, 6 and/or 7 or a sequence having at least 80% identity after optimum alignment with sequences SEQ ID Nos. 4, 5, 6 and/or 7.

The term “monoclonal antibody” as used herein is not limited to antibodies produced through hybridoma technology. A monoclonal antibody is derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof. In many uses of the present disclosure, including in vivo use of anti-SLIT2 antibodies, binding fragments and/or derivatives in humans, chimeric, primatized, humanized, or human antibodies can suitably be used.

“Humanized” forms of non-human (e.g., murine) antibodies are chimeric immunoglobulins that contain minimal sequences derived from non-human immunoglobulin. In general, a humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody can also comprise at least a portion of an immunoglobulin constant region (Fc), typically that of a human immunoglobulin consensus sequence. Methods of antibody humanization are known in the art.

“Human antibodies” are antibodies having the amino acid sequence of a human immunoglobulin and include antibodies isolated from human immunoglobulin libraries or from animals transgenic for one or more human immunoglobulin and that do not express endogenous immunoglobulins. Human antibodies can be made by a variety of methods known in the art including phage display methods using antibody libraries derived from human immunoglobulin sequences. Human antibodies can also be produced using transgenic mice which are incapable of expressing functional endogenous immunoglobulins but which can express human immunoglobulin genes. Fully human antibodies that recognize a selected epitope can be generated using a technique referred to as “guided selection.” In this approach, a selected non-human monoclonal antibody, e.g., a mouse antibody, is used to guide the selection of a completely human antibody recognizing the same.

Anti-SLIT2 antibodies may comprise full-length (intact) antibody molecules, as well as antigen binding fragments and/or derivatives that are capable of specifically binding SLIT2. Examples of antibody binding fragments include by way of example and not limitation, Fab, Fab′, F(ab′)₂, Fv fragments, single chain Fv fragments and single domain fragments.

A Fab fragment contains the constant domain of the light chain and the first constant domain (CH₂) of the heavy chain. Fab′ fragments differ from Fab fragments by the addition of a few residues at the carboxyl terminus of the heavy chain CH₂ domain including one or more cysteines from the antibody hinge region. F(ab′) fragments are produced by cleavage of the disulfide bond at the hinge cysteines of the F(ab′)₂ pepsin digestion product. Additional chemical couplings of antibody fragments are known to those of ordinary skill in the art. Fab and F(ab′)₂ fragments lack the Fc fragment of intact antibody, clear more rapidly from the circulation of animals, and may have less non-specific tissue binding than an intact antibody.

An “Fv” fragment is the minimum fragment of an antibody that contains a complete target recognition and binding site. This region consists of a dimer of one heavy and one light chain variable domain in a tight, non-covalent association (V_{H}-V_{L} dimer). It is in this configuration that the three CDRs of each variable domain interact to define an antigen binding site on the surface of the V_{H}-V_{L} dimer. Often, the six CDRs confer antigen binding specificity upon the antibody. However, in some instances even a single variable domain (or half of an Fv comprising only three CDRs specific for a target) may have the ability to recognize and bind antigen, although at a lower affinity than the entire binding site.

“Single-chain Fv” or “scFv” antibody binding fragments comprise the V_{H} and V_{L} domains of an antibody, where these domains are present in a single polypeptide chain. Generally, the Fv polypeptide further comprises a polypeptide linker between the V_{H} and V_{L} domains which enables the scFv to form the desired structure for antigen binding.

Antibodies, binding fragments, and/or derivatives composing the anti-SLIT2 antibodies may include modifications and/or mutations that alter the properties of the antibodies and/or fragments, such as those that increase half-life, increase or decrease antigen-dependent cellular cytotoxicity (“ADCC”), etc., as is known in the art.

“Single domain antibodies” are composed of a single V_{H} or V_{L} domains which exhibit sufficient affinity to SLIT2. In one example embodiment, the single domain antibody is a camelized antibody.

The anti-SLIT2 antibodies may also be bispecific antibodies. Bispecific antibodies comprised of monoclonal, often human or humanized, antibodies that have binding specificities for two different epitopes on the same or different antigens. In the present disclosure, one of the binding specificities can be directed towards SLIT2, the other can be for any other antigen, e.g., for a cell-surface protein, receptor, receptor subunit, tissue-specific antigen, virally derived protein, virally encoded envelope protein, bacterially derived protein, or bacterial surface protein, etc. For example, in one embodiment, one of the binding specificities is directed towards SLIT2, the other is directed to SLIT2.

Anti-SLIT2 antibodies may also be derivatized. Derivatized antibodies are typically modified by glycosylation, acetylation, pegylation, phosphorylation, amidation, derivatization by known protecting/blocking groups, proteolytic cleavage, linkage to a cellular ligand or other protein. Any of numerous chemical modifications may be carried out by known techniques, including, but not limited to, specific chemical cleavage, acetylation, formylation, metabolic synthesis of tunicamycin, etc. Additionally, the derivative may contain one or more non-natural amino acids.

Anti-SLIT2 antibodies, binding fragments, and/or derivatives thereof may be antibodies or fragments whose sequences have been modified to, for example, alter at least one constant region-mediated biological effector function. For example, in some embodiments, an anti-SLIT2 antibody may be modified to reduce at least one constant region-mediated biological effector function relative to the unmodified antibody, e.g., reduced binding to the Fc receptor (FcγR). FcγR binding may be reduced by mutating the immunoglobulin constant region segment of the antibody at particular regions necessary for FcγR interactions. Reducing FcγR binding may also reduce other effector functions which rely on FcγR interactions, such as opsonization, phagocytosis and ADCC.

According to the present disclosure, heavy and light chain variable framework regions were examined for mutation options and locations to enhance activity, reduce immunogenicity risk, remove glycosylation sites, and/or reduce deamidation risk using, for example, abYsis v4.0. Structural analysis confirmed substitutions that are understood not to affect stability. A variety of mutations locations and substitutions were developed and are described herein.

In particular embodiments, an anti-SLIT2 antibody is mutated such that at least one of amino acid residues 13, 78 and/or 86 of the heavy chain variable region is substituted alone, or in any combinations thereof, such as at positions 13 and 78, or at positions 13 and 86, or at positions 78 and 86, or at positions 13, 78, and 86. For position 13, the substituting amino acid residue may be any amino acid residue other than asparagine, including, but not limited to, alanine, cysteine, aspartic acid, glutamine, glutamic acid, phenylalanine, glycine, histidine, isoleucine, lysine, leucine, methionine, asparagine, proline, arginine, serine, valine, tryptophan, or tyrosine. For position 78, the substituting amino acid residue may be an amino acid residue other than threonine, including but not limited to, alanine, cysteine, phenylalanine, glycine, histidine, isoleucine, lysine, asparagine, proline, serine, or valine. For position 86, the substituting amino acid residue may be an amino acid residue other than asparagine, including, aspartic acid. Nucleic acid variants encoding the amino acid substitution mutants described herein are also contemplated.

In the same or additional embodiments, an anti-SLIT2 antibody is mutated such that at least one of amino acid residues 31, 72, 74, and/or 76 of the light chain variable region is substituted alone, or in any combinations thereof, such as at positions 31, 72, 74, or 76, or at positions 72 and 74, or at positions 72 and 76, or at positions 74 and 76, or at positions 31 and 72, or at positions 31 and 74, or at positions 31 and 76, or at positions 31, 72 and 76, or at positions 31, 74 and 76, or at positions 31, 72, 74 and 76. For position 31, the substituting amino acid residue may be an amino acid residue other than serine, including, alanine. For position 72, the substituting amino acid residue may be any amino acid residue other than asparagine, including but not limited to, alanine, cysteine, aspartic acid, glutamine, glutamic acid, phenylalanine, glycine, histidine, isoleucine, lysine, leucine, methionine, asparagine, proline, arginine, serine, valine, tryptophan, or tyrosine. For position 74, the substituting amino acid residue may be any amino acid residue other than asparagine, including but not limited to, alanine, cysteine, aspartic acid, glutamine, glutamic acid, phenylalanine, glycine, histidine, isoleucine, lysine, leucine, methionine, asparagine, proline, arginine, serine, valine, tryptophan, or tyrosine. For position 76, the substituting amino acid residue may be any amino acid residue other than serine, including but not limited to, alanine, cysteine, aspartic acid, glutamine, glutamic acid, phenylalanine, glycine, histidine, isoleucine, lysine, leucine, methionine, asparagine, proline, arginine, serine, valine, tryptophan, or tyrosine. Nucleic acid variants encoding the amino acid substitution mutants described herein are also contemplated.

Anti-SLIT2 substitution mutant antibodies were produced using the mutated heavy and/or light chain variable regions described herein. Each of the recited mutated or non-mutated light chain variable regions is paired with the recited mutated or non-mutated light chain variable regions in a comprehensive set of pairings and their binding activity is evaluated according to the methods described herein. Exemplary Anti-SLIT2 substitution mutant antibodies having a selection of these pairings are set forth below in Table 9 (VH-VL pairing).

TABLE 9

| Antibody ID | VH name | Mutation on VH | VL name | Mutation on VL |
|---|---|---|---|---|
| 75C1_1.1 | 75C1_HC_Mut_1.1 | N13Q | 75C1_LC_Mut_1.1 | N72T, N74T |
| 75C1_1.2 | 75C1_HC_Mut_1.2 | N13Q, T78V | 75C1_LC_Mut_1.1 | N72T, N74T |
| 75C1_1.3 | 75C1_HC_Mut_1.3 | N13Q, T78V, N86D | 75C1_LC_Mut_1.1 | N72T, N74T |
| 75C1_1.4 | 75C1_HC_Mut_1.4 | N13Q, N86D | 75C1_LC_Mut_1.1 | N72T, N74T |
| 75C1_1.5 | 75C1_HC_Mut_1.1 | N13Q | 75C1_LC_Mut_1.2 | N72T |
| 75C1_1.6 | 75C1_HC_Mut_1.2 | N13Q, T78V | 75C1_LC_Mut_1.2 | N72T |
| 75C1_1.7 | 75C1_HC_Mut_1.3 | N13Q, T78V, N86D | 75C1_LC_Mut_1.2 | N72T |
| 75C1_1.8 | 75C1_HC_Mut_1.4 | N13Q, N86D | 75C1_LC_Mut_1.2 | N72T |
| 75C1_2.1 | 75C1_HC_Mut_1.1 | N13Q | 75C1_LC_Mut_2.1 | N72T, N74T, S31A |
| 75C1_2.2 | 75C1_HC_Mut_1.2 | N13Q, T78V | 75C1_LC_Mut_2.1 | N72T, N74T, S31A |
| 75C1_2.3 | 75C1_HC_Mut_1.3 | N13Q, T78V, N86D | 75C1_LC_Mut_2.1 | N72T, N74T, S31A |
| 75C1_2.4 | 75C1_HC_Mut_1.4 | N13Q, N86D | 75C1_LC_Mut_2.1 | N72T, N74T, S31A |
| 75C1_2.5 | 75C1_HC_Mut_1.1 | N13Q | 75C1_LC_Mut_2.2 | N72T, S31A |
| 75C1_2.6 | 75C1_HC_Mut_1.2 | N13Q, T78V | 75C1_LC_Mut_2.2 | N72T, S31A |
| 75C1_2.7 | 75C1_HC_Mut_1.3 | N13Q, T78V, N86D | 75C1_LC_Mut_2.2 | N72T, S31A |
| 75C1_2.8 | 75C1_HC_Mut_1.4 | N13Q, N86D | 75C1_LC_Mut_2.2 | N72T, S31A |
| 75C1_HCM1 | 75C1_HC_Mut_1.1 | N13Q | 75C1_LC | none |
| 75C1_HCM2 | 75C1_HC_Mut_1.2 | N13Q, T78V | 75C1_LC | none |
| 75C1_HCM3 | 75C1_HC_Mut_1.3 | N13Q, T78V, N86D | 75C1_LC | none |
| 75C1_HCM4 | 75C1_HC_Mut_1.4 | N13Q, N86D | 75C1_LC | none |
| 75C1_LCM1 | 75C1_HC | none | 75C1_LC_Mut_1.1 | N72T, N74T |
| 75C1_LCM2 | 75C1_HC | none | 75C1_LC_Mut_1.2 | N72T |
| 75C1_LCM3 | 75C1_HC | none | 75C1_LC_Mut_2.1 | N72T, N74T, S31A |
| 75C1_LCM4 | 75C1_HC | none | 75C1_LC_Mut_2.2 | N72T, S31A |

The exemplary Anti-SLIT2 substitution mutant antibodies (i.e., Antibody ID) of Table 9 may be provided in any of the variety of forms contemplated according to the methods and compositions of the present disclosure. For example, they may be provided as full length antibodies or fragments, including functional fragments. They may also include or lack all of a part of the constant region or a portion of the variable region of the light and/or variable chains. The specific amino acid and nucleic acid sequences for each of the CDRs, variable domains and full length for each of the Antibody IDs is readily known based on the present disclosure. For example, Antibody ID Nos 75C1_1.2 and 75C1_2.3, as identified above in Table 9, include the CDR, variable domain and full length amino acid and nucleic acid sequences set forth in Table 10.

TABLE 10

| | 75C1_1.2 | | 75C1_2.3 | |
|---|---|---|---|---|
| | Amino acid | Nucleic acid | Amino acid | Nucleic acid |
| | sequence | sequence | sequence | sequence |
| Heavy chain CDR1 | SEQ ID NO: 1 | SEQ ID NO: 17 | SEQ ID NO: 1 | SEQ ID NO: 17 |
| Heavy chain CDR2 | SEQ ID NO: 2 | SEQ ID NO: 18 | SEQ ID NO: 2 | SEQ ID NO: 18 |
| Heavy chain CDR3 | SEQ ID NO: 3 | SEQ ID NO: 19 | SEQ ID NO: 3 | SEQ ID NO: 19 |
| Light chain CDR1 | SEQ ID NO: 4 | SEQ ID NO: 20 | SEQ ID NO: 7 | SEQ ID NO: 23 |
| Light chain CDR2 | SEQ ID NO: 5 | SEQ ID NO: 21 | SEQ ID NO: 5 | SEQ ID NO: 21 |
| Light chain CDR3 | SEQ ID NO: 6 | SEQ ID NO: 22 | SEQ ID NO: 6 | SEQ ID NO: 22 |
| Heavy chain | SEQ ID NO: 25 | SEQ ID NO: 33 | SEQ ID NO: 26 | SEQ ID NO: 34 |
| variable | | | | |
| Light chain | SEQ ID NO: 28 | SEQ ID NO: 36 | SEQ ID NO: 30 | SEQ ID NO: 38 |
| variable | | | | |
| Full length Heavy | SEQ ID NO: 45 | SEQ ID NO: 53 | SEQ ID NO: 46 | SEQ ID NO: 54 |
| Chain | | | | |
| Full length light | SEQ ID NO: 48 | SEQ ID NO: 56 | SEQ ID NO: 50 | SEQ ID NO: 58 |
| chain | | | | |

Anti-SLIT2 antibodies may have low levels of, or lack, fucose. Antibodies lacking fucose have been correlated with enhanced ADCC activity, especially at low doses of antibody. Methods of preparing fucose-less antibodies include growth in rat myeloma YB2/0 cells (ATCC CRL 1662). YB2/0 cells express low levels of FUT8 mRNA, which encodes α-1,6-fucosyltransferase, an enzyme necessary for fucosylation of polypeptides.

An anti-SLIT2 antibody, binding fragment, and/or derivative may have one or more amino acids inserted into one or more of its hypervariable regions.

Anti-SLIT2 antibodies, binding fragments, and/or derivatives with high affinity for SLIT2 may be desirable for therapeutic uses. Accordingly, the present disclosure contemplates anti-SLIT2 antibodies, binding fragments, and/or derivatives having a high binding affinity to SLIT2. In specific embodiments, the antibodies, binding fragments, and/or derivatives bind SLIT2 with an affinity of at least about 100 nM, but may exhibit higher affinity, for example, at least about 90 nM, 80 nM, 70 nM, 60 nM, 50 nM, 40 nM, 30 nM, 25 nM, 20 nM, 15 nM, 10 nM, 7 nM, 6 nM, 5 nM, 4 nM, 3 nM, 2 nM, 1 nM, 0.1 nM, 0.01 nM, or even higher. In some embodiments, the antibodies bind SLIT2 with an affinity in the range of about 1 pM to about 100 nM, or an affinity ranging between any of the foregoing values.

Affinity of antibodies, binding fragments, and/or derivatives for SLIT2 can be determined using techniques well known in the art or described herein, such as for example, but not by way of limitation, ELISA, biolayer interferometry, isothermal titration calorimetry (ITC), surface plasmon resonance, flow cytometry or fluorescent polarization assays. In one embodiment, affinity refers to apparent affinity EC50 values measured according to the examples.

In the context of this disclosure, anti-SLIT2 antibodies, binding fragments, and/or derivatives can serve at least two different purposes. In some embodiments, the anti-SLIT2 antibodies, binding fragments, and/or derivatives are used for diagnostic purposes, assisting in and guiding patient selection. For example, these anti-SLIT2 antibodies, binding fragments, and/or derivatives can be used for diagnostic or treatment purposes. One of ordinary skill in the art is familiar with the techniques for selecting a particular antibody for diagnostic purposes to assay for the levels of SLIT2 protein expression in tissues. Typically, the samples are scored under one or more scoring guides, including IHC scores of 0/1+/2+/3+ or H-scores. Companion diagnostics exist for a variety of other FDA approved treatments and are within the level of ordinary skill. The FDA maintains a list of FDA-approved companion diagnostic tests at, for example, www.fda.gov/.

By CDR regions or CDR(s), it is intended to indicate the hypervariable regions of the heavy and light chains of the immunoglobulins as defined by Chothia.

Three heavy chain CDRs and 3 light chain CDRs exist. The term CDR or CDRs is used here in order to indicate, according to the case, one of these regions or several, or even the whole, of these regions which contain the majority of the amino acid residues responsible for the binding by affinity of the antibody for the antigen or the epitope which it recognizes.

By “percentage of identity” between two nucleic acid or amino acid sequences in the sense of the present invention, it is intended to indicate a percentage of nucleotides or of identical amino acid residues between the two sequences to be compared, obtained after the best alignment (optimum alignment), this percentage being purely statistical and the differences between the two sequences being distributed randomly and over their entire length. The comparisons of sequences between two nucleic acid or amino acid sequences are traditionally carried out by comparing these sequences after having aligned them in an optimum manner, said comparison being able to be carried out by segment or by “comparison window”. The optimum alignment of the sequences for the comparison can be carried out, in addition to manually, using the local homology algorithm of Smith and Waterman, Ad. App. Math. 2:482 (1981), by means of the local homology algorithm of Neddleman and Wunsch, J. Mol. Biol. 48: 443 (1970), by means of the similarity search method of Pearson and Lipman, Proc. Natl. Acad. Sci. USA 85:2444 (1988), by means of computer software using these algorithms (GAP, BESTFIT, FASTA and TFASTA in the Wisconsin Genetics Software Package, Genetics Computer Group, 575 Science Dr., Madison, Wis., or else by BLAST N or BLAST P comparison software).

The percentage of identity between two nucleic acid or amino acid sequences is determined by comparing these two sequences aligned in an optimum manner and in which the nucleic acid or amino acid sequence to be compared can comprise additions or deletions with respect to the reference sequence for an optimum alignment between these two sequences. The percentage of identity is calculated by determining the number of identical positions for which the nucleotide or the amino acid residue is identical between the two sequences, by dividing this number of identical positions by the total number of positions in the comparison window and by multiplying the result obtained by 100 in order to obtain the percentage of identity between these two sequences.

For example, it is possible to use the BLAST program, “BLAST 2 sequences” (Tatusova et al., “Blast 2 sequences—a new tool for comparing protein and nucleotide sequences”, FEMS Microbiol Lett. 174:247-250) available on the site http://www.ncbi.nlm.nih.gov/gorf/b12.html.

By amino acid sequence having at least 80%, preferably 85%, 90%, 95% and 98% identity with a reference amino acid sequence, those having, with respect to the reference sequence, certain modifications, in particular a deletion, addition or substitution of at least one amino acid, a truncation or an elongation are preferred. In the case of a substitution of one or more consecutive or nonconsecutive amino acid(s), the substitutions are preferred in which the substituted amino acids are replaced by “equivalent” amino acids. The expression “equivalent amino acids” is aimed here at indicating any amino acid capable of being substituted with one of the amino acids of the base structure without, however, essentially modifying the biological activities of the corresponding antibodies and such as will be defined later, especially in the examples. These equivalent amino acids can be determined either by relying on their structural homology with the amino acids which they replace, or on results of comparative trials of biological activity between the different antibodies capable of being carried out.

By way of example, mention is made of the possibilities of substitution capable of being carried out without resulting in a profound modification of the biological activity of the corresponding modified antibody.

As one non-limiting example, the following Table 11 provides substitution possibilities conceivable with a conservation of the biological activity of the modified antibody. The reverse substitutions are also, of course, possible in the same conditions.

TABLE 11

| Original Residue | Substitution(s) |
|---|---|
| Ala (A) | Val, Gly, Pro |
| Arg (R) | Lys, His |
| Asn (N) | Gln |
| Asp (D) | Glu |
| Cys (C) | Ser |
| Gln (Q) | Asn |
| Glu (G) | Asp |
| Gly (G) | Ala |
| His (H) | Arg |
| Ile (I) | Leu |
| Leu (L) | Ile, Val, Met |
| Lys (K) | Arg |
| Met (M) | Leu |
| Phe (F) | Tyr |
| Pro (P) | Ala |
| Ser (S) | Thr, Cys |
| Thr (T) | Ser |
| Trp (W) | Tyr |
| Tyr (Y) | Phe, Trp |
| Val (V) | Leu, Ala |

It must be understood here that the present disclosure does not relate to the antibodies in natural form, that is to say they are not in their natural environment but that they have been able to be isolated or obtained by purification from natural sources, or else obtained by genetic recombination, or by chemical synthesis, and that they can then contain unnatural amino acids as will be described further on.

The present disclosure also concerns more particularly a chimeric and/or a humanized divalent antibody, or any divalent functional fragment or derivative, with an antagonistic activity relative to the SLIT2-SLIT2 interaction and related activity.

According to the first approach, the present antibody may be defined by its heavy chain or light chain sequence. More particularly, the antibody of the present disclosure, or one of its functional fragments or derivatives, is characterized in that it comprises a heavy chain and/or light chain comprising at least one CDR chosen from CDRs comprising the amino acid sequences specified in Table 3, 75C1_LC_MUT 2.1, 2.2 refers to antibodies with S31A mutation derived from 75C1. According to a preferred aspect, the antibody of the present disclosure, or one of its functional fragments or derivatives, comprises a heavy chain comprising at least one, preferably two, and most preferably three, CDR(s) chosen from CDR-H1, CDR-H2 and CDR-H3, wherein: CDR-H1 comprises the amino acid sequence SEQ ID NO: 1; CDR-H2 comprises the amino acid sequence SEQ ID NO: 2; and/or CDR-H3 comprises the amino acid sequence SEQ ID NO: 3. According to another preferred aspect, the antibody of the present disclosure, or one of its functional fragments or derivatives, comprises a light chain comprising at least one, preferably two, and most preferably three, CDR(s) chosen from CDR-L1, CDR-L2 and CDR-L3, wherein: CDR-L1 comprises the amino acid sequence SEQ ID NO: 4 or 7; CDR-L2 comprises the amino acid sequence SEQ ID NO: 5; and/or CDR-L3 comprises the amino acid sequence SEQ ID NO: 6.

In another approach, antibodies, or functional fragments or derivatives, of the present disclosure are defined by heavy chain variable sequence, if present, as set forth in Tables 5 or 7.

In another approach, the antibodies, or functional fragments or derivatives, of the present disclosure are defined by light chain sequence, if present, as set forth in Tables 5 or 7.

A transgenic mouse hybridoma capable of secreting monoclonal antibodies according to the present disclosure created using the herein described methods and reagents.

In the present application, null effector functions are preferred, though in certain embodiments ADCC and CDC effector functions are provided with the antibodies described herein.

The skilled artisan will recognize that effector functions include, for example, C1q binding; complement dependent cytotoxicity (CDC); Fc receptor binding; antibody-dependent cell-mediated cytotoxicity (ADCC); phagocytosis; and down regulation of cell surface receptors (e.g., B cell receptor; BCR).

The antibodies according to the present disclosure, are preferably specific monoclonal antibodies, especially of murine, chimeric or humanized origin, which can be obtained according to the standard methods well known to the person skilled in the art.

In general, for the preparation of monoclonal antibodies or their functional fragments or derivatives it is possible to refer to techniques which are described in particular in the manual “Antibodies” (Harlow and Lane, Antibodies: A Laboratory Manual, Cold Spring Harbor Laboratory, Cold Spring Harbor N.Y., pp. 726, 1988) or to the technique of preparation from hybridomas described by Kohler and Milstein (Nature, 256:495-497, 1975).

Certain monoclonal antibodies according to the present disclosure can be obtained, for example, from an animal cell immunized against SLIT2, or one of its fragments containing an epitope specifically recognized by said antibodies according to the present disclosure. SLIT2, or one of its fragments, can be produced according to the usual working methods, by genetic recombination starting with a nucleic acid sequence contained in the cDNA sequence coding for SLIT2 or by peptide synthesis starting from a sequence of amino acids comprised in the peptide sequence of SLIT2.

The monoclonal antibodies according to the present disclosure can, for example, be purified on an affinity column on which SLIT2 or one of its fragments containing an epitope specifically recognized by said monoclonal antibodies according to the present disclosure has previously been immobilized. More particularly, the antibodies can be purified by chromatography on protein A and/or G, followed or not followed by ion-exchange chromatography aimed at eliminating the residual protein contaminants as well as the DNA and the LPS, in itself followed or not followed by exclusion chromatography on Sepharose™ gel in order to eliminate the potential aggregates due to the presence of dimers or of other multimers. In an even more preferred manner, the whole of these techniques can be used simultaneously or successively.

The antibody of the present disclosure, or a divalent functional fragment or derivative thereof, consists preferably of a chimeric antibody.

The antibody, or a functional fragment or derivative thereof, may comprise a heavy chain variable domain of sequence comprising the amino acid sequence SEQ ID NOS: 1, 2, 3 and/or 8 or a sequence having at least 80%, 81%, 82%, 83%, 84%, 85%, 86%, 87%, 88%, 89%, 90%, 95%, 96%, 97%, 98% or 99% identity after optimum alignment with the sequence SEQ ID NOS: 1, 2, 3 and/or 8

The antibody, or a functional fragment or derivative thereof, may also or alternatively comprise a light chain variable domain of sequence comprising the amino acid sequence SEQ ID NO: 4, 5, 6, 7 and/or 9 or a sequence having at least 80%, 81%, 82%, 83%, 84%, 85%, 86%, 87%, 88%, 89%, 90%, 95%, 96%, 97%, 98% or 99% identity after optimum alignment with the sequence SEQ ID NOS. 4, 5, 6, 7 and/or 9.

The antibody of the present disclosure, or a divalent functional fragment or derivative thereof, most frequently comprises a human antibody.

In one embodiment, the human monoclonal antibodies are produced by a hybridoma which includes a B cell obtained from a transgenic non-human animal, e.g., a transgenic mouse (e.g., transgenic mouse available from Acemab (Hunan, China), XENOMOUSE®, etc.), having a genome comprising a human heavy chain transgene and a light chain transgene fused to an immortalized cell.

The antibody, or a functional fragment or derivative thereof, may comprise a human heavy chain variable domain of sequence comprising the amino acid sequence SEQ ID NOS. 1, 2, 3 and/or 8 or a sequence having at least 80%, 81%, 82%, 83%, 84%, 85%, 86%, 87%, 88%, 89%, 90%, 95%, 96%, 97%, 98% or 99% identity after optimum alignment with the sequence SEQ ID NO: 1, 2, 3 and/or 8.

The antibody, or a functional fragment or derivative thereof, may also or alternatively comprise a human light chain variable domain of sequence comprising the amino acid sequence SEQ ID NOS: 4, 5, 6, 7 and/or 9 or a sequence having at least 80%, 81%, 82%, 83%, 84%, 85%, 86%, 87%, 88%, 89%, 90%, 95%, 96%, 97%, 98% or 99% identity after optimum alignment with the sequence SEQ ID NOS: 4, 5, 6, 7 and/or 9.

Any other technique known by the man skill in the art, such as phage display technique, can also be used for the generation of human antibody according to the present disclosure.

The antibody of the present disclosure, or a divalent functional fragment or derivative thereof, may also comprise a humanized antibody.

The humanized antibodies according to the present disclosure or their fragments can be prepared by techniques known to the person skilled in the art (such as, for example, those described in the documents Singer et al., J. Immun. 150:2844-2857, 1992; Mountain et al., Biotechnol. Genet. Eng. Rev., 10:1-142, 1992; or Bebbington et al., Bio/Technology, 10:169-175, 1992). Other humanization method is known by the skilled artisan as, for example, the “CDR Grafting” method.

The antibody, or a functional fragment or derivative thereof, may comprise a humanized heavy chain variable domain of sequence comprising the amino acid sequence SEQ ID NO: 1, 2, 3 and/or 8 or a sequence having at least 80%, 81%, 82%, 83%, 84%, 85%, 86%, 87%, 88%, 89%, 90%, 95%, 96%, 97%, 98% or 99% identity after optimum alignment with the sequence SEQ ID NO: 1, 2, 3 and/or 8.

The antibody, or a functional fragment or derivative thereof, may also or alternatively comprise a humanized light chain variable domain of sequence comprising the amino acid sequence SEQ ID NOS: 4, 5, 6, 7 and/or 9 or a sequence having at least 80%, 81%, 82%, 83%, 84%, 85%, 86%, 87%, 88%, 89%, 90%, 95%, 96%, 97%, 98% or 99% identity after optimum alignment with the sequence SEQ ID NOS: 4, 5, 6, 7 and/or 9.

“Functional fragment” of an antibody according to the present disclosure refers to an antibody fragment, such as Fv, scFv (sc for single chain), Fab, F(ab)₂, Fab′, scFv-Fc fragments or diabodies, or any fragment of which the half-life time would have been increased by chemical modification, such as the addition of poly(alkylene) glycol such as poly(ethylene) glycol (“PEGylation”) (pegylated fragments called Fv-PEG, scFv-PEG, Fab-PEG, F(ab′)₂-PEG or Fab′-PEG) (“PEG” for Poly(Ethylene) Glycol), or by incorporation in a liposome, said fragments having at least one of the characteristic CDRs of sequence SEQ ID Nos. 1 to 7 according to the present disclosure, and, especially, in that it is capable of exerting in a general manner an even partial activity of the antibody from which it is descended, such as in particular the capacity to recognize and to bind to SLIT2, and, if necessary, to modulate the activity of SLIT2, and/or modulate the interaction of SLIT2 with ROBO2.

Preferably, said functional fragments are constituted or comprise a partial sequence of the heavy or light variable chain of the antibody from which they are derived, the partial sequence being sufficient to retain the same specificity of binding as the antibody from which it is descended and a sufficient affinity, preferably at least equal to 1/100, in a more preferred manner to at least 1/10, of that of the antibody from which it is descended, with respect to SLIT2. Such a functional fragment will contain at the minimum 5 amino acids, preferably 6, 7, 8, 9, 10, 12, 15, 25, 50 and 100 consecutive amino acids of the sequence of the antibody from which it is descended.

Preferably, these functional fragments will be fragments of Fv, scFv, Fab, F(ab′)₂, F(ab′), scFv-Fc type or diabodies, which generally have the same specificity of binding as the antibody from which they are descended. In a more preferred embodiment of the invention, these fragments are selected among divalent fragments such as F(ab′)₂ fragments. According to the present invention, antibody fragments of the invention can be obtained starting from antibodies such as described above by methods such as digestion by enzymes, such as pepsin or papain and/or by cleavage of the disulfide bridges by chemical reduction. In another manner, the antibody fragments comprised in the present invention can be obtained by techniques of genetic recombination likewise well known to the person skilled in the art or else by peptide synthesis by means of, for example, automatic peptide synthesizers such as those supplied by the company Applied Biosystems, etc.

“Divalent fragment” refers to any antibody fragments comprising two arms and, more particularly, F(ab′)₂ fragments.

“Derivatives” of an antibody according to the present disclosure refers to a binding protein comprising a protein scaffold and at least one of the CDRs selected from the original antibody to maintain the binding capacity. Such compounds are well known by the skilled artisan and will be described in more details in the following specification. More particularly, the antibody, or one of its functional fragments or derivatives, according to the present disclosure is characterized in that said derivative consists in a binding protein comprising a scaffold on which at least one CDR has been grafted for the conservation of the original antibody paratopic recognizing properties.

One or several sequences of the 6 CDR sequences of one or more antibody or one or more of the heavy and/or light chain of the antibodies described in the present disclosure can be presented on a protein scaffold. In such case, the protein scaffold reproduces the protein backbone with appropriate folding of the grafted CDR(s), thus allowing it (or them) to maintain their antigen paratopic recognizing properties. The skilled artisan will know how to select the protein scaffold on which at least one CDR selected from the original antibody could be grafted. More particularly, it is known that, to be selected, such scaffold should display several features as follow (Skerra A., J. Mol. Recogn., 13, 2000, 167-187): phylogenetically good conservation; robust architecture with a well-known three-dimensional molecular organization (such as, for example, crystallography or NMR); small size; no or only low degree of post-translational modifications; and easy to produce, express and purify.

Such protein scaffold can be, but without limitation, structure selected from the group consisting in fibronectin and preferentially the tenth fibronectin type Ill domain (FNfn10), lipocalin, anticalin, the protein Z derivative from the domain B of staphylococcal protein A, thioredoxin A or any protein with repeated domain such as “ankyrin repeat,” “armadillo repeat”, “leucin-rich repeat” and/or “tetratricopeptide repeat.” Also optionally the scaffold is a derivative from toxins (such as, for example, scorpion, insect, plant or mollusc toxins) or protein inhibitors of neuronal nitric oxyde synthase (PIN). As above mentioned, one or several sequences of the 6 CDR sequences of one or more antibody or one or more of the heavy and/or light chain of the antibodies described in the present disclosure can be presented on a protein scaffold. In one set of embodiments, though without limitation, the skilled artisan selects at least one or more CDR from the heavy chain. The selection of the CDR(s) of interest will be evident for the skilled artisan with known methods.

As an evidence, these examples are not limitative and any other scaffold known or described must be included in the present specification.

According to still another aspect, the present disclosure relates to isolated nucleic acids, characterized in that it is chosen from the following nucleic acids: a nucleic acid, DNA or RNA, coding for an antibody, or one of its functional fragments or derivatives, according to the present disclosure, where the antibody comprises or has a sequence selected from the group consisting of the sequences SEQ ID Nos. 1-9, 12-13, 24-31, 40-41, or 44-51.

By nucleic acid, nucleic or nucleic acid sequence, polynucleotide, oligonucleotide, polynucleotide sequence, nucleotide sequence, terms which will be employed indifferently in the present invention, it is intended to indicate a precise linkage of nucleotides, which are modified or unmodified, allowing a fragment or a region of a nucleic acid to be defined, containing or not containing unnatural nucleotides, and being able to correspond just as well to a double-stranded DNA, a single-stranded DNA as to the transcription products of said DNAs.

It must also be understood here that the present disclosure does not concern the nucleotide sequences in their natural chromosomal environment, that is to say in the natural state. It concerns sequences which have been isolated and/or purified, that they have been selected directly or indirectly, for example by copy, their environment having been at least partially modified. It is thus likewise intended to indicate here the isolated nucleic acids obtained by genetic recombination by means, for example, of host cells or obtained by chemical synthesis.

A hybridization under conditions of high stringency signifies that the temperature conditions and ionic strength conditions are chosen in such a way that they allow the maintenance of the hybridization between two fragments of complementary DNA. By way of illustration, conditions of high stringency of the hybridization step for the purposes of defining the polynucleotide fragments described above are advantageously the following.

The DNA-DNA or DNA-RNA hybridization is carried out in two steps: (1) prehybridization at 42° C. for 3 hours in phosphate buffer (20 mM, pH 7.5) containing 5×SSC (1×SSC corresponds to a 0.15 M NaCl+0.015 M sodium citrate solution), 50% of formamide, 7% of sodium dodecyl sulfate (SDS), 10×Denhardt's, 5% of dextran sulfate and 1% of salmon sperm DNA; (2) actual hybridization for 20 hours at a temperature dependent on the size of the probe (i.e.: 42° C., for a probe size>100 nucleotides) followed by 2 washes of 20 minutes at 20° C. in 2×SSC+2% of SDS, 1 wash of 20 minutes at 20° C. in 0.1×SSC+0.1% of SDS. The last wash is carried out in 0.1×SSC+0.1% of SDS for 30 minutes at 60° C. for a probe size>100 nucleotides. The hybridization conditions of high stringency described above for a polynucleotide of defined size can be adapted by the person skilled in the art for oligonucleotides of greater or smaller size, according to the teaching of Sambrook et al. (1989, Molecular cloning: a laboratory manual. 2nd Ed. Cold Spring Harbor).

The present disclosure relates and is directed to one or more vector comprising a nucleic acid according to the present disclosure.

The present disclosure includes as described elsewhere herein cloning and/or expression vectors which contain a nucleotide sequence according to the present disclosure.

The present disclosure relates and is directed to host cells transformed by or comprising a vector according to the present disclosure.

The present disclosure relates and is directed to animals, except man, which comprise at least one cell transformed according to the present disclosure.

According to another aspect, a subject of the present disclosure is a process for production of an antibody, or one of its functional fragments according to the present disclosure, for example, as described herein.

The present disclosure also concerns the antibody of the invention as a medicament.

The present disclosure likewise concerns a pharmaceutical composition comprising by way of active principle a compound consisting of an antibody, or one of its functional fragments according to the present disclosure, preferably mixed with an excipient and/or a pharmaceutically acceptable vehicle.

In some embodiments, the heavy chain of an anti-SLIT2 antibody comprises or consists of a variable region and CDRs (CDR sequences disclosed as SEQ ID Nos. 1, 2, 3, respectively, in order of appearance) (SEQ ID NO: 8):

QVQLVQSGAEVKNPGSSVKVSCKASGGSFRSYTVSWVRQAPGQGLEWMG
GIIPIFGTANYAQKFQGRVTITADESTDTTYMDLSSLKSENTAVYYCAT
GSGWYTFDYWGQGTLVTVSS

And, the light chain comprises or consists of a variable region and CDR sequences (disclosed as SEQ ID Nos. 4, 5, 6, respectively, in order of appearance) (SEQ ID NO: 9):

DIQMTQSPSSLSASIGDRVTITCRASQNINSYLNWYQQKPGKAPKFLIY
AASNLQSGVPSRFSGSGSGTDFNLNISSLQPEDFAIYYCQQSYRSPPTF
GGGTTVEIK

Examples of suitable heavy and light chain constant regions are provided below. In some embodiments, the anti-SLIT2 antibody comprises a heavy chain variable region comprising SEQ ID NO: 8 linked to any heavy chain constant region; and a light chain variable region comprising SEQ ID NO: 9 linked to any light chain constant region. Examples of suitable heavy and light chain constant regions are provided below.

In some embodiments, an anti-SLIT2, binding fragment, and/or derivative composing an anti-SLIT2 antibody is an IgG1.

In some embodiments, an anti-SLIT2 antibody heavy chain comprises or consists of (full-length sequence disclosed as SEQ ID NO: 12):

QVQLVQSGAEVKNPGSSVKVSCKASGGSFRSYTVSWVRQAPGQGLEWMG
GIIPIFGTANYAQKFQGRVTITADESTDTTYMDLSSLKSENTAVYYCAT
GSGWYTFDYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPEAAGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALGAPIEKTISKAKGQ
PREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKS
LSLSPGK

In some embodiments, an anti-SLIT2 antibody light chain comprises or consists of (full-length sequence disclosed as SEQ ID NO: 13):

DIQMTQSPSSLSASIGDRVTITCRASQNINSYLNWYQQKPGKAPKFLIY
AASNLQSGVPSRFSGSGSGTDFNLNISSLQPEDFAIYYCQQSYRSPPTF
GGGTTVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

In one embodiment, the anti-SLIT2 antibody heavy chain is encoded by the following nucleotide sequence (full-length sequence disclosed as SEQ ID NO: 14):

CAGGTGCAGCTGGTGCAGTCTGGGGCTGAGGTGAAGAACCCTGGGTCCTCGGTGAAGGTCTCCTGCAAGGCTTCT
GGAGGCTCCTTCAGGAGCTATACTGTCAGTTGGGTGCGACAGGCCCCTGGACAAGGGCTTGAGTGGATGGGAGG
GATCATCCCTATCTTTGGTACAGCAAACTACGCACAGAAGTTCCAGGGCAGAGTCACGATTACCGCGGACGAATCC
ACGGACACAACCTACATGGACCTGAGCAGCCTGAAATCTGAAAACACGGCCGTGTATTATTGTGCGACAGGTAGT
GGCTGGTACACCTTTGACTACTGGGGCCAGGGAACCCTGGTCACCGTCTCCTCAGCTAGCACCAAGGGCCCATCG
GTCTTCCCCCTGGCACCCTCCTCCAAGAGCACCTCTGGGGGCACAGCGGCCCTGGGCTGCCTGGTCAAGGACTACT
TCCCCGAACCGGTGACGGTGTCGTGGAACTCAGGCGCCCTGACCAGCGGCGTGCACACCTTCCCGGCTGTCCTAC
AGTCCTCAGGACTCTACTCCCTCAGCAGCGTGGTGACCGTGCCCTCCAGCAGCTTGGGCACCCAGACCTACATCTG
CAACGTGAATCACAAGCCCAGCAACACCAAGGTGGACAAGAAAGTTGAGCCCAAATCTTGTGACAAAACTCACAC
ATGCCCACCGTGCCCAGCACCTGAAGCTGCAGGGGGACCGTCAGTCTTCCTCTTCCCCCCAAAACCCAAGGACACC
CTCATGATCTCCCGGACCCCTGAGGTCACATGCGTGGTGGTGGACGTGAGCCACGAAGACCCTGAGGTCAAGTTC
AACTGGTACGTGGACGGCGTGGAGGTGCATAATGCCAAGACAAAGCCGCGGGAGGAGCAGTACAACAGCACGT
ACCGTGTGGTCAGCGTCCTCACCGTCCTGCACCAGGACTGGCTGAATGGCAAGGAGTACAAGTGCAAGGTCTCCA
ACAAAGCCCTCGGAGCCCCCATCGAGAAAACCATCTCCAAAGCCAAAGGGCAGCCCCGAGAACCACAGGTGTACA
CCCTGCCCCCATCCCGGGATGAGCTAACCAAGAACCAGGTCAGCCTGACCTGCCTGGTCAAAGGCTTCTATCCCAG
CGACATCGCCGTGGAGTGGGAGAGCAATGGGCAGCCGGAGAACAACTACAAGACCACGCCTCCCGTGCTGGACT
CCGACGGCTCCTTCTTCCTCTACAGCAAGCTCACCGTGGACAAGAGCAGGTGGCAGCAGGGGAACGTCTTCTCAT
GCTCCGTGATGCATGAGGCTCTGCACAACCACTACACGCAGAAGAGCCTCTCCCTGTCTCCGGGTAAA

In one embodiment, the anti-SLIT2 antibody light chain is encoded by the following nucleotide sequence (full-length sequence disclosed as SEQ ID NO: 15):

GACATCCAGATGACCCAGTCTCCATCCTCCCTGTCTGCATCTATAGGAGACAGAGTCACCATCACTTGCCGGGCGA
GTCAAAACATTAACAGCTATTTAAATTGGTATCAGCAGAAACCAGGGAAAGCCCCTAAGTTCCTGATCTATGCTGC
ATCCAATTTGCAAAGTGGGGTCCCATCAAGGTTCAGTGGCAGTGGATCTGGGACAGATTTCAATCTCAACATCAGC
AGTCTGCAGCCTGAAGATTTTGCAATTTACTACTGTCAACAGAGCTATAGAAGTCCTCCCACTTTCGGCGGAGGGA
CCACGGTGGAGATTAAACGTACGGTGGCGGCGCCATCTGTCTTCATCTTCCCGCCATCTGATGAGCAGTTGAAATC
TGGAACTGCCTCTGTTGTGTGCCTGCTGAATAACTTCTATCCCAGAGAGGCCAAAGTACAGTGGAAGGTGGATAA
CGCCCTCCAATCGGGTAACTCCCAGGAGAGTGTCACAGAGCAGGACAGCAAGGACAGCACCTACAGCCTCAGCA
GCACCCTGACGCTGAGCAAAGCAGACTACGAGAAACACAAAGTCTACGCCTGCGAAGTCACCCATCAGGGCCTGA
GCTCGCCCGTCACAAAGAGCTTCAACAGGGGAGAGTGT

In some embodiments, an anti-SLIT2 substitution mutant antibody heavy chain constant region (CH1+CH2+CH3) comprises or consists of the following amino acid sequence (SEQ ID NO: 40):

ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALGAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

In some embodiments, an anti-SLIT2 substitution mutant antibody light chain constant region comprises or consists of the following amino acid sequence (SEQ ID NO: 41): RTVAAPSVFIFPPSDEQLKSGTASVVCLLNNFYPREAKVQWKVDNALQSGNSQESVTEQDSKDSTYSLSSTLTLSKADYE KHKVYACEVTHQGLSSPVTKSFNRGEC

In one embodiment, the anti-SLIT2 antibody substitution mutant heavy chain constant region (CH1+CH2+CH3) is encoded by the following nucleotide sequence (SEQ ID NO: 42):

GCTAGCACCAAGGGCCCATCGGTCTTCCCCCTGGCACCCTCCTCCAAGAGCACCTCTGGGGGCACAGCGG
CCCTGGGCTGCCTGGTCAAGGACTACTTCCCCGAACCGGTGACGGTGTCGTGGAACTCAGGCGCCCTGAC
CAGCGGCGTGCACACCTTCCCGGCTGTCCTACAGTCCTCAGGACTCTACTCCCTCAGCAGCGTGGTGACC
GTGCCCTCCAGCAGCTTGGGCACCCAGACCTACATCTGCAACGTGAATCACAAGCCCAGCAACACCAAGG
TGGACAAGAAAGTTGAGCCCAAATCTTGTGACAAAACTCACACATGCCCACCGTGCCCAGCACCTGAAGC
TGCAGGGGGACCGTCAGTCTTCCTCTTCCCCCCAAAACCCAAGGACACCCTCATGATCTCCCGGACCCCT
GAGGTCACATGCGTGGTGGTGGACGTGAGCCACGAAGACCCTGAGGTCAAGTTCAACTGGTACGTGGACG
GCGTGGAGGTGCATAATGCCAAGACAAAGCCGCGGGAGGAGCAGTACAACAGCACGTACCGTGTGGTCAG
CGTCCTCACCGTCCTGCACCAGGACTGGCTGAATGGCAAGGAGTACAAGTGCAAGGTCTCCAACAAAGCC
CTCGGAGCCCCCATCGAGAAAACCATCTCCAAAGCCAAAGGGCAGCCCCGAGAACCACAGGTGTACACCC
TGCCCCCATCCCGGGATGAGCTAACCAAGAACCAGGTCAGCCTGACCTGCCTGGTCAAAGGCTTCTATCC
CAGCGACATCGCCGTGGAGTGGGAGAGCAATGGGCAGCCGGAGAACAACTACAAGACCACGCCTCCCGTG
CTGGACTCCGACGGCTCCTTCTTCCTCTACAGCAAGCTCACCGTGGACAAGAGCAGGTGGCAGCAGGGGA
ACGTCTTCTCATGCTCCGTGATGCATGAGGCTCTGCACAACCACTACACGCAGAAGAGCCTCTCCCTGTC
TCCGGGTAAA

In one embodiment, the anti-SLIT2 antibody substitution mutant light chain constant region is encoded by the following nucleotide sequence (SEQ ID NO: 43):

CGTACGGTGGCGGCGCCATCTGTCTTCATCTTCCCGCCATCTGATGAGC
AGTTGAAATCTGGAACTGCCTCTGTTGTGTGCCTGCTGAATAACTTCTA
TCCCAGAGAGGCCAAAGTACAGTGGAAGGTGGATAACGCCCTCCAATCG
GGTAACTCCCAGGAGAGTGTCACAGAGCAGGACAGCAAGGACAGCACCT
ACAGCCTCAGCAGCACCCTGACGCTGAGCAAAGCAGACTACGAGAAACA
CAAAGTCTACGCCTGCGAAGTCACCCATCAGGGCCTGAGCTCGCCCGTC
ACAAAGAGCTTCAACAGGGGAGAGTGT

For a full length anti-SLIT2 antibody substitution mutant antibody heavy chain amino acid or nucleic acid sequence, the anti-SLIT2 antibody substitution mutant heavy constant region disclosed above and herein is combined with any of the anti-SLIT2 antibody substitution mutant heavy chain variable region amino acid or nucleic acid sequences, respectively. For a full length anti-SLIT2 antibody substitution mutant antibody light chain amino acid or nucleic acid sequence, the anti-SLIT2 antibody substitution mutant light constant region disclosed above and herein is combined with any of the anti-SLIT2 antibody substitution mutant light chain variable region amino acid or nucleic acid sequences, respectively. Exemplary full length heavy and light chain amino acid sequences are set forth in Table 12. Exemplary full length heavy and light chain nucleic acid sequences are set forth below Table 12.

TABLE 12

| Name | Full length amino acid sequence |
|---|---|
| 75C1_HC_MUT_1.1 | SEQ ID NO: 44 |
| (FULL LENGTH HEAVY | QVQLVQSGAEVKQPGSSVKVSCKASGGSFRSYTVSWVR |
| CHAIN) | QAPGQGLEWMGGIIPIFGTANYAQKFQGRVTITADEST |
| | DTTYMDLSSLKSENTAVYYCATGSGWYTFDYWGQGTLV |
| | TVSSASTKGPSVFPLAPSSKSTSGGTAALGCLVKDYFP |
| | EPVTVSWNSGALTSGVHTFPAVLQSSGLYSLSSVVTVP |
| | SSSLGTQTYICNVNHKPSNTKVDKKVEPKSCDKTHTCP |
| | PCPAPEAAGGPSVFLFPPKPKDTLMISRTPEVTCVVVD |
| | VSHEDPEVKFNWYVDGVEVHNAKTKPREEQYNSTYRVV |
| | SVLTVLHQDWLNGKEYKCKVSNKALGAPIEKTISKAKG |
| | QPREPQVYTLPPSRDELTKNQVSLTCLVKGFYPSDIAV |
| | EWESNGQPENNYKTTPPVLDSDGSFFLYSKLTVDKSRW |
| | QQGNVFSCSVMHEALHNHYTQKSLSLSPGK |
| 75C1_HC_MUT_1.2 | SEQ ID NO: 45 |
| (FULL LENGTH HEAVY | QVQLVQSGAEVKQPGSSVKVSCKASGGSFRSYTVSWVR |
| CHAIN) | QAPGQGLEWMGGIIPIFGTANYAQKFQGRVTITADEST |
| | DTVYMDLSSLKSENTAVYYCATGSGWYTFDYWGQGTLV |
| | TVSSASTKGPSVFPLAPSSKSTSGGTAALGCLVKDYFP |
| | EPVTVSWNSGALTSGVHTFPAVLQSSGLYSLSSVVTVP |
| | SSSLGTQTYICNVNHKPSNTKVDKKVEPKSCDKTHTCP |
| | PCPAPEAAGGPSVFLFPPKPKDTLMISRTPEVTCVVVD |
| | VSHEDPEVKFNWYVDGVEVHNAKTKPREEQYNSTYRVV |
| | SVLTVLHQDWLNGKEYKCKVSNKALGAPIEKTISKAKG |
| | QPREPQVYTLPPSRDELTKNQVSLTCLVKGFYPSDIAV |
| | EWESNGQPENNYKTTPPVLDSDGSFFLYSKLTVDKSRW |
| | QQGNVFSCSVMHEALHNHYTQKSLSLSPGK |
| 75C1_HC_MUT_1.3 | SEQ ID NO: 46 |
| (FULL LENGTH HEAVY | QVQLVQSGAEVKQPGSSVKVSCKASGGSFRSYTVSWVR |
| CHAIN) | QAPGQGLEWMGGIIPIFGTANYAQKFQGRVTITADEST |
| | DTVYMDLSSLKSEDTAVYYCATGSGWYTFDYWGQGTLV |
| | TVSSASTKGPSVFPLAPSSKSTSGGTAALGCLVKDYFP |
| | EPVTVSWNSGALTSGVHTFPAVLQSSGLYSLSSVVTVP |
| | SSSLGTQTYICNVNHKPSNTKVDKKVEPKSCDKTHTCP |
| | PCPAPEAAGGPSVFLFPPKPKDTLMISRTPEVTCVVVD |
| | VSHEDPEVKFNWYVDGVEVHNAKTKPREEQYNSTYRVV |
| | SVLTVLHQDWLNGKEYKCKVSNKALGAPIEKTISKAKG |
| | QPREPQVYTLPPSRDELTKNQVSLTCLVKGFYPSDIAV |
| | EWESNGQPENNYKTTPPVLDSDGSFFLYSKLTVDKSRW |
| | QQGNVFSCSVMHEALHNHYTQKSLSLSPGK |
| 75C1_HC_MUT_1.4 | SEQ ID NO: 47 |
| (FULL LENGTH HEAVY | QVQLVQSGAEVKQPGSSVKVSCKASGGSFRSYTVSWVR |
| CHAIN) | QAPGQGLEWMGGIIPIFGTANYAQKFQGRVTITADEST |
| | DTTYMDLSSLKSEDTAVYYCATGSGWYTFDYWGQGTLV |
| | TVSSASTKGPSVFPLAPSSKSTSGGTAALGCLVKDYFP |
| | EPVTVSWNSGALTSGVHTFPAVLQSSGLYSLSSVVTVP |
| | SSSLGTQTYICNVNHKPSNTKVDKKVEPKSCDKTHTCP |
| | PCPAPEAAGGPSVFLFPPKPKDTLMISRTPEVTCVVVD |
| | VSHEDPEVKFNWYVDGVEVHNAKTKPREEQYNSTYRVV |
| | SVLTVLHQDWLNGKEYKCKVSNKALGAPIEKTISKAKG |
| | QPREPQVYTLPPSRDELTKNQVSLTCLVKGFYPSDIAV |
| | EWESNGQPENNYKTTPPVLDSDGSFFLYSKLTVDKSRW |
| | QQGNVFSCSVMHEALHNHYTQKSLSLSPGK |
| 75C1_LC_MUT_1.1 | SEQ ID NO: 48 |
| (FULL LENGTH LIGHT | DIQMTQSPSSLSASIGDRVTITCRASQNINSYLNWYQQ |
| CHAIN) | KPGKAPKFLIYAASNLQSGVPSRFSGSGSGTDFTLTIS |
| | SLQPEDFAIYYCQQSYRSPPTFGGGTTVEIKRTVAAPS |
| | VFIFPPSDEQLKSGTASVVCLLNNFYPREAKVQWKVDN |
| | ALQSGNSQESVTEQDSKDSTYSLSSTLTLSKADYEKHK |
| | VYACEVTHQGLSSPVTKSFNRGEC |
| 75C1_LC_MUT_1.2 | SEQ ID NO: 49 |
| (FULL LENGTH LIGHT | DIQMTQSPSSLSASIGDRVTITCRASQNINSYLNWYQQ |
| CHAIN) | KPGKAPKFLIYAASNLQSGVPSRFSGSGSGTDFTLNIS |
| | SLQPEDFAIYYCQQSYRSPPTFGGGTTVEIKRTVAAPS |
| | VFIFPPSDEQLKSGTASVVCLLNNFYPREAKVQWKVDN |
| | ALQSGNSQESVTEQDSKDSTYSLSSTLTLSKADYEKHK |
| | VYACEVTHQGLSSPVTKSFNRGEC |
| 75C1_LC_MUT_2.1 | SEQ ID NO: 50 |
| (FULL LENGTH LIGHT | DIQMTQSPSSLSASIGDRVTITCRASQNINAYLNWYQQ |
| CHAIN) | KPGKAPKFLIYAASNLQSGVPSRFSGSGSGTDFTLTIS |
| | SLQPEDFAIYYCQQSYRSPPTFGGGTTVEIKRTVAAPS |
| | VFIFPPSDEQLKSGTASVVCLLNNFYPREAKVQWKVDN |
| | ALQSGNSQESVTEQDSKDSTYSLSSTLTLSKADYEKHK |
| | VYACEVTHQGLSSPVTKSFNRGEC |
| 75C1_LC_MUT_2.2 | SEQ ID NO: 51 |
| (FULL LENGTH LIGHT | DIQMTQSPSSLSASIGDRVTITCRASQNINAYLNWYQQ |
| CHAIN) | KPGKAPKFLIYAASNLQSGVPSRFSGSGSGTDFTLNIS |
| | SLQPEDFAIYYCQQSYRSPPTFGGGTTVEIKRTVAAPS |
| | VFIFPPSDEQLKSGTASVVCLLNNFYPREAKVQWKVDN |
| | ALQSGNSQESVTEQDSKDSTYSLSSTLTLSKADYEKHK |
| | VYACEVTHQGLSSPVTKSFNRGEC |

75C1_HC_MUT_1.1 (FULL LENGTH HEAVY CHAIN)
SEQ ID NO: 52
CAGGTGCAGCTGGTGCAGTCTGGGGCTGAGGTGAAGCAACCTGGGTCCTCGGTGAAGGTCTCCTGCAAGGCTTCT
GGAGGCTCCTTCAGGAGCTATACTGTCAGTTGGGTGCGACAGGCCCCTGGACAAGGGCTTGAGTGGATGGGAGG
GATCATCCCTATCTTTGGTACAGCAAACTACGCACAGAAGTTCCAGGGCAGAGTCACGATTACCGCGGACGAATCC
ACGGACACAACCTACATGGACCTGAGCAGCCTGAAATCTGAAAACACGGCCGTGTATTATTGTGCGACAGGTAGT
GGCTGGTACACCTTTGACTACTGGGGCCAGGGAACCCTGGTCACCGTCTCCTCAGCTAGCACCAAGGGCCCATCG
GTCTTCCCCCTGGCACCCTCCTCCAAGAGCACCTCTGGGGGCACAGCGGCCCTGGGCTGCCTGGTCAAGGACTACT
TCCCCGAACCGGTGACGGTGTCGTGGAACTCAGGCGCCCTGACCAGCGGCGTGCACACCTTCCCGGCTGTCCTAC
AGTCCTCAGGACTCTACTCCCTCAGCAGCGTGGTGACCGTGCCCTCCAGCAGCTTGGGCACCCAGACCTACATCTG
CAACGTGAATCACAAGCCCAGCAACACCAAGGTGGACAAGAAAGTTGAGCCCAAATCTTGTGACAAAACTCACAC
ATGCCCACCGTGCCCAGCACCTGAAGCTGCAGGGGGACCGTCAGTCTTCCTCTTCCCCCCAAAACCCAAGGACACC
CTCATGATCTCCCGGACCCCTGAGGTCACATGCGTGGTGGTGGACGTGAGCCACGAAGACCCTGAGGTCAAGTTC
AACTGGTACGTGGACGGCGTGGAGGTGCATAATGCCAAGACAAAGCCGCGGGAGGAGCAGTACAACAGCACGT
ACCGTGTGGTCAGCGTCCTCACCGTCCTGCACCAGGACTGGCTGAATGGCAAGGAGTACAAGTGCAAGGTCTCCA
ACAAAGCCCTCGGAGCCCCCATCGAGAAAACCATCTCCAAAGCCAAAGGGCAGCCCCGAGAACCACAGGTGTACA
CCCTGCCCCCATCCCGGGATGAGCTAACCAAGAACCAGGTCAGCCTGACCTGCCTGGTCAAAGGCTTCTATCCCAG
CGACATCGCCGTGGAGTGGGAGAGCAATGGGCAGCCGGAGAACAACTACAAGACCACGCCTCCCGTGCTGGACT
CCGACGGCTCCTTCTTCCTCTACAGCAAGCTCACCGTGGACAAGAGCAGGTGGCAGCAGGGGAACGTCTTCTCAT
GCTCCGTGATGCATGAGGCTCTGCACAACCACTACACGCAGAAGAGCCTCTCCCTGTCTCCGGGTAAA
75C1_HC_MUT_1.2 (FULL LENGTH HEAVY CHAIN)
SEQ ID NO: 53
CAGGTGCAGCTGGTGCAGTCTGGGGCTGAGGTGAAGCAACCTGGGTCCTCGGTGAAGGTCTCCTGCAAGGCTTCT
GGAGGCTCCTTCAGGAGCTATACTGTCAGTTGGGTGCGACAGGCCCCTGGACAAGGGCTTGAGTGGATGGGAGG
GATCATCCCTATCTTTGGTACAGCAAACTACGCACAGAAGTTCCAGGGCAGAGTCACGATTACCGCGGACGAATCC
ACGGACACAGTATACATGGACCTGAGCAGCCTGAAATCTGAAAACACGGCCGTGTATTATTGTGCGACAGGTAGT
GGCTGGTACACCTTTGACTACTGGGGCCAGGGAACCCTGGTCACCGTCTCCTCAGCTAGCACCAAGGGCCCATCG
GTCTTCCCCCTGGCACCCTCCTCCAAGAGCACCTCTGGGGGCACAGCGGCCCTGGGCTGCCTGGTCAAGGACTACT
TCCCCGAACCGGTGACGGTGTCGTGGAACTCAGGCGCCCTGACCAGCGGCGTGCACACCTTCCCGGCTGTCCTAC
AGTCCTCAGGACTCTACTCCCTCAGCAGCGTGGTGACCGTGCCCTCCAGCAGCTTGGGCACCCAGACCTACATCTG
CAACGTGAATCACAAGCCCAGCAACACCAAGGTGGACAAGAAAGTTGAGCCCAAATCTTGTGACAAAACTCACAC
ATGCCCACCGTGCCCAGCACCTGAAGCTGCAGGGGGACCGTCAGTCTTCCTCTTCCCCCCAAAACCCAAGGACACC
CTCATGATCTCCCGGACCCCTGAGGTCACATGCGTGGTGGTGGACGTGAGCCACGAAGACCCTGAGGTCAAGTTC
AACTGGTACGTGGACGGCGTGGAGGTGCATAATGCCAAGACAAAGCCGCGGGAGGAGCAGTACAACAGCACGT
ACCGTGTGGTCAGCGTCCTCACCGTCCTGCACCAGGACTGGCTGAATGGCAAGGAGTACAAGTGCAAGGTCTCCA
ACAAAGCCCTCGGAGCCCCCATCGAGAAAACCATCTCCAAAGCCAAAGGGCAGCCCCGAGAACCACAGGTGTACA
CCCTGCCCCCATCCCGGGATGAGCTAACCAAGAACCAGGTCAGCCTGACCTGCCTGGTCAAAGGCTTCTATCCCAG
CGACATCGCCGTGGAGTGGGAGAGCAATGGGCAGCCGGAGAACAACTACAAGACCACGCCTCCCGTGCTGGACT
CCGACGGCTCCTTCTTCCTCTACAGCAAGCTCACCGTGGACAAGAGCAGGTGGCAGCAGGGGAACGTCTTCTCAT
GCTCCGTGATGCATGAGGCTCTGCACAACCACTACACGCAGAAGAGCCTCTCCCTGTCTCCGGGTAAA
75C1_HC_MUT_1.3 (FULL LENGTH HEAVY CHAIN)
SEQ ID NO: 54
CAGGTGCAGCTGGTGCAGTCTGGGGCTGAGGTGAAGCAACCTGGGTCCTCGGTGAAGGTCTCCTGCAAGGCTTCT
GGAGGCTCCTTCAGGAGCTATACTGTCAGTTGGGTGCGACAGGCCCCTGGACAAGGGCTTGAGTGGATGGGAGG
GATCATCCCTATCTTTGGTACAGCAAACTACGCACAGAAGTTCCAGGGCAGAGTCACGATTACCGCGGACGAATCC
ACGGACACAGTATACATGGACCTGAGCAGCCTGAAATCTGAAGATACGGCCGTGTATTATTGTGCGACAGGTAGT
GGCTGGTACACCTTTGACTACTGGGGCCAGGGAACCCTGGTCACCGTCTCCTCAGCTAGCACCAAGGGCCCATCG
GTCTTCCCCCTGGCACCCTCCTCCAAGAGCACCTCTGGGGGCACAGCGGCCCTGGGCTGCCTGGTCAAGGACTACT
TCCCCGAACCGGTGACGGTGTCGTGGAACTCAGGCGCCCTGACCAGCGGCGTGCACACCTTCCCGGCTGTCCTAC
AGTCCTCAGGACTCTACTCCCTCAGCAGCGTGGTGACCGTGCCCTCCAGCAGCTTGGGCACCCAGACCTACATCTG
CAACGTGAATCACAAGCCCAGCAACACCAAGGTGGACAAGAAAGTTGAGCCCAAATCTTGTGACAAAACTCACAC
ATGCCCACCGTGCCCAGCACCTGAAGCTGCAGGGGGACCGTCAGTCTTCCTCTTCCCCCCAAAACCCAAGGACACC
CTCATGATCTCCCGGACCCCTGAGGTCACATGCGTGGTGGTGGACGTGAGCCACGAAGACCCTGAGGTCAAGTTC
AACTGGTACGTGGACGGCGTGGAGGTGCATAATGCCAAGACAAAGCCGCGGGAGGAGCAGTACAACAGCACGT
ACCGTGTGGTCAGCGTCCTCACCGTCCTGCACCAGGACTGGCTGAATGGCAAGGAGTACAAGTGCAAGGTCTCCA
ACAAAGCCCTCGGAGCCCCCATCGAGAAAACCATCTCCAAAGCCAAAGGGCAGCCCCGAGAACCACAGGTGTACA
CCCTGCCCCCATCCCGGGATGAGCTAACCAAGAACCAGGTCAGCCTGACCTGCCTGGTCAAAGGCTTCTATCCCAG
CGACATCGCCGTGGAGTGGGAGAGCAATGGGCAGCCGGAGAACAACTACAAGACCACGCCTCCCGTGCTGGACT
CCGACGGCTCCTTCTTCCTCTACAGCAAGCTCACCGTGGACAAGAGCAGGTGGCAGCAGGGGAACGTCTTCTCAT
GCTCCGTGATGCATGAGGCTCTGCACAACCACTACACGCAGAAGAGCCTCTCCCTGTCTCCGGGTAAA
75C1_HC_MUT_1.4 (FULL LENGTH HEAVY CHAIN)
SEQ ID NO: 55
CAGGTGCAGCTGGTGCAGTCTGGGGCTGAGGTGAAGCAACCTGGGTCCTCGGTGAAGGTCTCCTGCAAGGCTTCT
GGAGGCTCCTTCAGGAGCTATACTGTCAGTTGGGTGCGACAGGCCCCTGGACAAGGGCTTGAGTGGATGGGAGG
GATCATCCCTATCTTTGGTACAGCAAACTACGCACAGAAGTTCCAGGGCAGAGTCACGATTACCGCGGACGAATCC
ACGGACACAACCTACATGGACCTGAGCAGCCTGAAATCTGAAGACACGGCCGTGTATTATTGTGCGACAGGTAGT
GGCTGGTACACCTTTGACTACTGGGGCCAGGGAACCCTGGTCACCGTCTCCTCAGCTAGCACCAAGGGCCCATCG
GTCTTCCCCCTGGCACCCTCCTCCAAGAGCACCTCTGGGGGCACAGCGGCCCTGGGCTGCCTGGTCAAGGACTACT
TCCCCGAACCGGTGACGGTGTCGTGGAACTCAGGCGCCCTGACCAGCGGCGTGCACACCTTCCCGGCTGTCCTAC
AGTCCTCAGGACTCTACTCCCTCAGCAGCGTGGTGACCGTGCCCTCCAGCAGCTTGGGCACCCAGACCTACATCTG
CAACGTGAATCACAAGCCCAGCAACACCAAGGTGGACAAGAAAGTTGAGCCCAAATCTTGTGACAAAACTCACAC
ATGCCCACCGTGCCCAGCACCTGAAGCTGCAGGGGGACCGTCAGTCTTCCTCTTCCCCCCAAAACCCAAGGACACC
CTCATGATCTCCCGGACCCCTGAGGTCACATGCGTGGTGGTGGACGTGAGCCACGAAGACCCTGAGGTCAAGTTC
AACTGGTACGTGGACGGCGTGGAGGTGCATAATGCCAAGACAAAGCCGCGGGAGGAGCAGTACAACAGCACGT
ACCGTGTGGTCAGCGTCCTCACCGTCCTGCACCAGGACTGGCTGAATGGCAAGGAGTACAAGTGCAAGGTCTCCA
ACAAAGCCCTCGGAGCCCCCATCGAGAAAACCATCTCCAAAGCCAAAGGGCAGCCCCGAGAACCACAGGTGTACA
CCCTGCCCCCATCCCGGGATGAGCTAACCAAGAACCAGGTCAGCCTGACCTGCCTGGTCAAAGGCTTCTATCCCAG
CGACATCGCCGTGGAGTGGGAGAGCAATGGGCAGCCGGAGAACAACTACAAGACCACGCCTCCCGTGCTGGACT
CCGACGGCTCCTTCTTCCTCTACAGCAAGCTCACCGTGGACAAGAGCAGGTGGCAGCAGGGGAACGTCTTCTCAT
GCTCCGTGATGCATGAGGCTCTGCACAACCACTACACGCAGAAGAGCCTCTCCCTGTCTCCGGGTAAA
75C1_LC_MUT_1.1 (FULL LENGTH LIGHT CHAIN)
SEQ ID NO: 56
GACATCCAGATGACCCAGTCTCCATCCTCCCTGTCTGCATCTATAGGAGACAGAGTCACCATCACTTGCCGGGCGA
GTCAAAACATTAACAGCTATTTAAATTGGTATCAGCAGAAACCAGGGAAAGCCCCTAAGTTCCTGATCTATGCTGC
ATCCAATTTGCAAAGTGGGGTCCCATCAAGGTTCAGTGGCAGTGGATCTGGGACAGATTTCACACTCACTATCAGC
AGTCTGCAGCCTGAAGATTTTGCAATTTACTACTGTCAACAGAGCTATAGAAGTCCTCCCACTTTCGGCGGAGGGA
CCACGGTGGAGATTAAACGTACGGTGGCGGCGCCATCTGTCTTCATCTTCCCGCCATCTGATGAGCAGTTGAAATC
TGGAACTGCCTCTGTTGTGTGCCTGCTGAATAACTTCTATCCCAGAGAGGCCAAAGTACAGTGGAAGGTGGATAA
CGCCCTCCAATCGGGTAACTCCCAGGAGAGTGTCACAGAGCAGGACAGCAAGGACAGCACCTACAGCCTCAGCA
GCACCCTGACGCTGAGCAAAGCAGACTACGAGAAACACAAAGTCTACGCCTGCGAAGTCACCCATCAGGGCCTGA
GCTCGCCCGTCACAAAGAGCTTCAACAGGGGAGAGTGT
75C1_LC_MUT_1.2 (FULL LENGTH LIGHT CHAIN)
SEQ ID NO: 57
GACATCCAGATGACCCAGTCTCCATCCTCCCTGTCTGCATCTATAGGAGACAGAGTCACCATCACTTGCCGGGCGA
GTCAAAACATTAACAGCTATTTAAATTGGTATCAGCAGAAACCAGGGAAAGCCCCTAAGTTCCTGATCTATGCTGC
ATCCAATTTGCAAAGTGGGGTCCCATCAAGGTTCAGTGGCAGTGGATCTGGGACAGATTTCACACTCAACATCAGC
AGTCTGCAGCCTGAAGATTTTGCAATTTACTACTGTCAACAGAGCTATAGAAGTCCTCCCACTTTCGGCGGAGGGA
CCACGGTGGAGATTAAACGTACGGTGGCGGCGCCATCTGTCTTCATCTTCCCGCCATCTGATGAGCAGTTGAAATC
TGGAACTGCCTCTGTTGTGTGCCTGCTGAATAACTTCTATCCCAGAGAGGCCAAAGTACAGTGGAAGGTGGATAA
CGCCCTCCAATCGGGTAACTCCCAGGAGAGTGTCACAGAGCAGGACAGCAAGGACAGCACCTACAGCCTCAGCA
GCACCCTGACGCTGAGCAAAGCAGACTACGAGAAACACAAAGTCTACGCCTGCGAAGTCACCCATCAGGGCCTGA
GCTCGCCCGTCACAAAGAGCTTCAACAGGGGAGAGTGT
75C1_LC_MUT_2.1 (FULL LENGTH LIGHT CHAIN)
SEQ ID NO: 58
GACATCCAGATGACCCAGTCTCCATCCTCCCTGTCTGCATCTATAGGAGACAGAGTCACCATCACTTGCCGGGCGA
GTCAAAACATTAACGCGTATTTAAATTGGTATCAGCAGAAACCAGGGAAAGCCCCTAAGTTCCTGATCTATGCTGC
ATCCAATTTGCAAAGTGGGGTCCCATCAAGGTTCAGTGGCAGTGGATCTGGGACAGATTTCACACTCACTATCAGC
AGTCTGCAGCCTGAAGATTTTGCAATTTACTACTGTCAACAGAGCTATAGAAGTCCTCCCACTTTCGGCGGAGGGA
CCACGGTGGAGATTAAACGTACGGTGGCGGCGCCATCTGTCTTCATCTTCCCGCCATCTGATGAGCAGTTGAAATC
TGGAACTGCCTCTGTTGTGTGCCTGCTGAATAACTTCTATCCCAGAGAGGCCAAAGTACAGTGGAAGGTGGATAA
CGCCCTCCAATCGGGTAACTCCCAGGAGAGTGTCACAGAGCAGGACAGCAAGGACAGCACCTACAGCCTCAGCA
GCACCCTGACGCTGAGCAAAGCAGACTACGAGAAACACAAAGTCTACGCCTGCGAAGTCACCCATCAGGGCCTGA
GCTCGCCCGTCACAAAGAGCTTCAACAGGGGAGAGTGT
75C1_LC_MUT_2.2 (FULL LENGTH LIGHT CHAIN)
SEQ ID NO: 59
GACATCCAGATGACCCAGTCTCCATCCTCCCTGTCTGCATCTATAGGAGACAGAGTCACCATCACTTGCCGGGCGA
GTCAAAACATTAACGCGTATTTAAATTGGTATCAGCAGAAACCAGGGAAAGCCCCTAAGTTCCTGATCTATGCTGC
ATCCAATTTGCAAAGTGGGGTCCCATCAAGGTTCAGTGGCAGTGGATCTGGGACAGATTTCACACTCAACATCAGC
AGTCTGCAGCCTGAAGATTTTGCAATTTACTACTGTCAACAGAGCTATAGAAGTCCTCCCACTTTCGGCGGAGGGA
CCACGGTGGAGATTAAACGTACGGTGGCGGCGCCATCTGTCTTCATCTTCCCGCCATCTGATGAGCAGTTGAAATC
TGGAACTGCCTCTGTTGTGTGCCTGCTGAATAACTTCTATCCCAGAGAGGCCAAAGTACAGTGGAAGGTGGATAA
CGCCCTCCAATCGGGTAACTCCCAGGAGAGTGTCACAGAGCAGGACAGCAAGGACAGCACCTACAGCCTCAGCA
GCACCCTGACGCTGAGCAAAGCAGACTACGAGAAACACAAAGTCTACGCCTGCGAAGTCACCCATCAGGGCCTGA
GCTCGCCCGTCACAAAGAGCTTCAACAGGGGAGAGTGT

Assays for competition include, but are not limited to, a radioactive material labeled immunoassay (RIA), an enzyme-linked immunosorbent assay (ELISA), a sandwich ELISA, flow cytometry assays and surface plasmon resonance assays.

In one exemplary embodiment of conducting an antibody competition assay between a reference antibody and a test antibody (irrespective of species or isotype), one may first label the reference with a detectable label, such as a fluorophore, biotin or an enzymatic, or radioactive label to enable subsequent detection. In this case, cells expressing SLIT2 or the extracellular domain of SLIT2 (or a subpart thereof), are incubated with unlabeled test antibody, labeled robo2-hFc is added, and the intensity of the bound label is measured. If the test antibody competes with the labeled reference antibody by binding to the same, proximal or overlapping epitope, the intensity of the detection signal will be decreased relative to a control reaction carried out without test antibody.

In a specific embodiment of this assay, the concentration of labeled robo2-hFc that yields 80% of maximal binding (“conc_{80%}”) under the assay conditions (e.g., a specified density of cells or a specified concentration of SLIT2/SLIT2 extracellular domain or subpart thereof) is first determined, and a competition assay is carried out with 10× concentration_{80%} of unlabeled test antibody and conc_{80%} of labeled reference antibody.

In another exemplary embodiment of conducting an ELISA competition assay, SLIT2 are incubated with a titration series of antibodies comprising increasing concentrations of unlabeled test antibody versus tagged SLIT2 protein antibody only. The his tagged reference SLIT2 protein is used at a fixed concentration X (for example, X=1 μg/ml) and the unlabeled test antibody is used in a range of concentrations (for example, from 10⁻⁴× to 100×). SLIT2 protein thereof is incubated with both unlabeled test antibody. The ELISA readout data is manifested as interaction between free Slit2 and Robo2 which is coated at the assay plate, where the signal of a sample carried out without unlabeled test antibody is assigned 100% binding. If a test antibody competes for binding SLIT2 with the coated Robo2 protein, an assay carried out with equal concentration of each (for example, 1 μg/mL of unlabeled test antibody and 1 μg/mL of labeled reference antibody) will yield an approximately 50% reduction in fluorescence intensity as compared to the 100% control, indicating approximately 50% binding. Use of a labeled reference antibody at a concentration of X and unlabeled test antibody that competes for binding SLIT2 at a concentration of 10X would yield an approximately 90% reduction in binding as compared to the 100% control, indicating approximately 10% binding.

The inhibition can be expressed as an inhibition constant, or Kᵢ, which is calculated according to the following formula:

Kᵢ=IC₅₀/(1+[reference Ab concentration]/K_{d}),
where IC₅₀ is the concentration of test antibody that yields a 50% reduction in binding of the reference antibody and K_{d} is the dissociation constant of the reference antibody, a measure of its affinity for SLIT2. Antibodies that compete with reference SLIT2 antibodies can have a Kᵢ from 10 pM to 100 nM under assay conditions described herein.

In various embodiments, a test antibody is considered to compete with a reference antibody if it decreases binding of the reference antibody to cells expressing SLIT2 or SLIT2/SLIT2 extracellular domain or subpart thereof by at least about 20% or more, for example, by at least about 20%, 30%, 40%, 50%, 60%, 70%, 80%, 90%, 95% or even more, or by a percentage ranging between any of the foregoing values, at a reference antibody concentration that is 80% of maximal binding under the specific assay conditions used, and a test antibody concentration that is 10-fold higher than the reference antibody concentration.

In various embodiments of a flow cytometry competition assay, a test antibody is considered to compete with a reference antibody if it decreases binding of the reference antibody to cells expressing SLIT2 by at least about 20% or more, for example, by at least about 20%, 30%, 40%, 50%, 60%, 70%, 80%, 90%, 95% or even more, or by a percentage ranging between any of the foregoing values, at a concentration of test antibody that is 10× greater than that of the reference antibody.

Detection of expression of SLIT2 generally involves contacting a biological sample (cells, tissue, or body fluid of an individual) with one or more anti-SLIT2 antibodies (optionally conjugated to detectable moiety) and detecting whether or not the sample is positive for SLIT2 expression, or whether the sample has altered (e.g., reduced or increased) expression as compared to a control sample. Methods for doing so are well known to one of ordinary skill in the art.

Anti-SLIT2 antibodies can be prepared by recombinant expression of immunoglobulin light and heavy chain genes in a host cell through methods well known to those of ordinary skill in the art. To express an antibody recombinantly, a host cell is transfected with one or more recombinant expression vectors carrying DNA fragments encoding the immunoglobulin light and heavy chains of the antibody such that the light and heavy chains are expressed in the host cell and, optionally, secreted into the medium in which the host cells are cultured, from which medium the antibodies can be recovered. Standard recombinant DNA methodologies are used to obtain antibody heavy and light chain genes, incorporate these genes into recombinant expression vectors and introduce the vectors into host cells, such as those described in Molecular Cloning; A Laboratory Manual, Second Edition (Sambrook, Fritsch and Maniatis (eds), Cold Spring Harbor, N.Y., 1989), Current Protocols in Molecular Biology (Ausubel, F. M. et al., eds., Greene Publishing Associates, 1989).

To generate nucleic acids encoding such anti-SLIT2 antibodies, DNA fragments encoding the light and heavy chain variable regions are first obtained. These DNAs can be obtained by amplification and modification of germline DNA or cDNA encoding light and heavy chain variable sequences, for example using the polymerase chain reaction (PCR). Germline DNA sequences for human heavy and light chain variable region genes are known in the art (See, e.g., the “VBASE” human germline sequence database; see also Kabat et al., 1991, Sequences of Proteins of Immunological Interest, Fifth Edition, U.S. Department of Health and Human Services, NIH Publication No. 91-3242; Tomlinson et al., 1992, J. Mol. Biol. 22T: 116-198; and Cox et al., 1994, Eur. J. Immunol. 24:827-836; the contents of each of which are incorporated herein by reference).

Once DNA fragments encoding anti-SLIT2 antibody-related V_{H} and V_{L} segments are obtained, these DNA fragments can be further manipulated by standard recombinant DNA techniques, for example to convert the variable region genes to full-length antibody chain genes, to Fab fragment genes or to a scFv gene. In these manipulations, a V_{L}- or V_{H}-encoding DNA fragment is operatively linked to another DNA fragment encoding another protein, such as an antibody constant region or a flexible linker. The term “operatively linked,” as used in this context, is intended to mean that the two DNA fragments are joined such that the amino acid sequences encoded by the two DNA fragments remain in-frame.

The isolated DNA encoding the V_{H} region can be converted to a full-length heavy chain gene by operatively linking the V_{H}-encoding DNA to another DNA molecule encoding heavy chain constant regions (CH₁, CH₂, CH₃ and, optionally, CH₄). The sequences of human heavy chain constant region genes are known in the art (See, e.g., Kabat et al., 1991, Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG₁, IgG₂, IgG₃, IgG₄, IgA, IgE, IgM or IgD constant region, but in certain embodiments is an IgG₁ or IgG₄ constant region. For a Fab fragment heavy chain gene, the V_{H}-encoding DNA can be operatively linked to another DNA molecule encoding only the heavy chain CH₁ constant region.

The isolated DNA encoding the V_{L} region can be converted to a full-length light chain gene (as well as a Fab light chain gene) by operatively linking the V_{L}-encoding DNA to another DNA molecule encoding the light chain constant region, CL. The sequences of human light chain constant region genes are known in the art (See, e.g., Kabat et al., 1991, Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region, but in certain embodiments is a kappa constant region.

To express the anti-SLIT2 antibodies, DNAs encoding partial or full-length light and heavy chains, obtained as described above, are inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vectors or, more typically, both genes are inserted into the same expression vector.

The antibody genes are inserted into the expression vector by standard methods (e.g., ligation of complementary restriction sites on the antibody gene fragment and vector, or blunt end ligation if no restriction sites are present). Prior to insertion of the anti-SLIT2 antibody-related light or heavy chain sequences, the expression vector can already carry antibody constant region sequences. For example, one approach to converting the anti-SLIT2 monoclonal antibody-related V_{H} and V_{L} sequences to full-length antibody genes is to insert them into expression vectors already encoding heavy chain constant and light chain constant regions, respectively, such that the V_{H} segment is operatively linked to the CH segment(s) within the vector and the V_{L} segment is operatively linked to the CL segment within the vector. Additionally, or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel, Gene Expression Technology: Methods in Enzymology 185, Academic Press, San Diego, Calif., 1990. It will be appreciated by those skilled in the art that the design of the expression vector, including the selection of regulatory sequences may depend on such factors as the choice of the host cell to be transformed, the level of expression of protein desired, etc. Suitable regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma.

Recombinant expression vectors of the disclosure can carry sequences in addition to the antibody chain genes and regulatory sequences, such as sequences that regulate replication of the vector in host cells (e.g., origins of replication) and selectable marker genes. Selectable marker genes facilitate selection of host cells into which the vector has been introduced. For example, typically a selectable marker gene confers resistance to drugs, such as G418, hygromycin or methotrexate, on a host cell into which the vector has been introduced. Suitable selectable marker genes include the dihydrofolate reductase (DHFR) gene (for use in DHFR-host cells with methotrexate selection/amplification) and the neo gene (for G418 selection). For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, lipofection, calcium-phosphate precipitation, DEAE-dextran transfection and the like.

It is possible to express anti-SLIT2 antibodies in either prokaryotic or eukaryotic host cells. In certain embodiments, expression of antibodies is performed in eukaryotic cells, e.g., mammalian host cells, of optimal secretion of a properly folded and immunologically active antibody. Exemplary mammalian host cells for expressing the recombinant antibodies of the disclosure include Chinese Hamster Ovary (CHO cells), NSO myeloma cells, COS cells and SP2 cells, among others. When recombinant expression vectors encoding antibody genes are introduced into mammalian host cells, the antibodies are produced by culturing the host cells for a period sufficient to allow for expression of the antibody in the host cells or secretion of the antibody into the culture medium in which the host cells are grown. Antibodies can be recovered from the culture medium using standard protein purification methods. Host cells can also be used to produce portions of intact antibodies, such as Fab fragments or scFv molecules. It is understood that variations on the above procedure are within the scope of the present disclosure. For example, it can be desirable to transfect a host cell with DNA encoding either the light chain or the heavy chain of an anti-SLIT2 antibody.

Recombinant DNA technology can also be used to remove some or all of the DNA encoding either or both of the light and heavy chains that is not necessary for binding to SLIT2. The molecules expressed from such truncated DNA molecules are also encompassed by the antibodies of the disclosure.

For recombinant expression of an anti-SLIT2 antibody, the host cell can be co-transfected with two expression vectors, the first vector encoding a heavy chain derived polypeptide and the second vector encoding a light chain derived polypeptide. The two vectors can contain identical selectable markers, or they can each contain a separate selectable marker. Alternatively, a single vector can be used which encodes both heavy and light chain polypeptides.

Once a nucleic acid encoding one or more portions of an anti-SLIT2 antibody is obtained, further alterations or mutations can be introduced into the coding sequence, for example to generate nucleic acids encoding antibodies with different CDR sequences, antibodies with reduced affinity to the Fc receptor, or antibodies of different subclasses.

Anti-SLIT2 antibodies, binding fragments, and/or derivatives thereof can also be produced by chemical synthesis (e.g., by the methods described in Solid Phase Peptide Synthesis, 2^{nd} ed., 1984 The Pierce Chemical Co., Rockford, Ill.). Variant antibodies can also be generated using a cell-free platform, See, e.g., Chu et al., Biochemia No. 2, 2001 (Roche Molecular Biologicals) and Murray et al., 2013, Current Opinion in Chemical Biology, 17:420-426.

Once an anti-SLIT2 antibody, binding fragment, and/or derivative has been produced by recombinant expression, it can be purified by any method known in the art for purification of an immunoglobulin molecule, for example, by chromatography (e.g., ion exchange, affinity, and sizing column chromatography), centrifugation, differential solubility, or by any other standard technique for the purification of proteins. Further, the anti-SLIT2 antibodies, binding fragments, and/or derivatives can be fused to heterologous polypeptide sequences described herein or otherwise known in the art to facilitate purification.

Once isolated, the anti-SLIT2 antibody, binding fragment, and/or derivative can, if desired, be further purified, e.g., by column chromatography. (see, e.g., Fisher, Laboratory Techniques In Biochemistry And Molecular Biology, Work and Burdon, eds., Elsevier, 1980), or by gel filtration chromatography on a Superdex™ 75 column (Pharmacia Biotech AB, Uppsala, Sweden).

The antibodies described herein may be in the form of compositions comprising the antibody and one or more carriers, excipients and/or diluents. The compositions may be formulated for specific uses, such as for veterinary uses or pharmaceutical uses in humans. The form of the composition (e.g., dry powder, liquid formulation, etc.) and the excipients, diluents and/or carriers used will depend upon the intended uses of the antibody for therapeutic uses, the mode of administration.

For therapeutic uses, the compositions may be supplied as part of a sterile, pharmaceutical composition that includes a pharmaceutically acceptable carrier. This composition can be in any suitable form (depending upon the desired method of administering it to a patient). The pharmaceutical composition can be administered to a patient by a variety of routes such as orally, transdermally, subcutaneously, intranasally, intravenously, intramuscularly, intratumorally, intrathecally, topically or locally. The most suitable route for administration in any given case will depend on the particular antibody, the subject, and the nature and severity of the disease and the physical condition of the subject. Typically, the pharmaceutical composition will be administered intravenously or subcutaneously.

Pharmaceutical compositions can be conveniently presented in unit dosage forms containing a predetermined amount of an antibody described herein per dose. The quantity of antibody included in a unit dose will depend on the disease being treated, as well as other factors as are well known in the art. Such unit dosages may be in the form of a lyophilized dry powder containing an amount of antibody suitable for a single administration, or in the form of a liquid. Dry powder unit dosage forms may be packaged in a kit with a syringe, a suitable quantity of diluent and/or other components useful for administration. Unit dosages in liquid form may be conveniently supplied in the form of a syringe pre-filled with a quantity of antibody suitable for a single administration.

The pharmaceutical compositions may also be supplied in bulk form containing quantities of ADC suitable for multiple administrations.

Pharmaceutical compositions may be prepared for storage as lyophilized formulations or aqueous solutions by mixing an antibody having the desired degree of purity with optional pharmaceutically-acceptable carriers, excipients or stabilizers typically employed in the art (all of which are referred to herein as “carriers”), i.e., buffering agents, stabilizing agents, preservatives, isotonifiers, non-ionic detergents, antioxidants, and other miscellaneous additives. See, Remington's Pharmaceutical Sciences, 16th edition (Osol, ed. 1980) and Remington: The Science and Practice of Pharmacy, 22^{nd} Edition (Edited by Allen, Loyd V. Jr., 2012). Such additives should be nontoxic to the recipients at the dosages and concentrations employed.

Buffering agents help to maintain the pH in the range which stabilizes the protein. They may be present at a wide variety of concentrations, but will typically be present in concentrations ranging from about 2 mM to about 50 mM. Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium gluconate mixture, gluconic acid-sodium hydroxide mixture, gluconic acid-potassium gluconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives may be added to retard microbial growth, and, can be added in amounts ranging from about 0.2%-1% (w/v). Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” can be added to ensure isotonicity of liquid compositions of the present disclosure and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinositol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, α-monothioglycerol and sodium thiosulfate; low molecular weight polypeptides (e.g., peptides of 10 residues or fewer); proteins such as human serum albumin, bovine serum albumin, gelatin or immunoglobulins; hydrophilic polymers, such as polyvinylpyrrolidone monosaccharides, such as xylose, mannose, fructose, glucose; disaccharides such as lactose, maltose, sucrose and trehalose; and trisaccacharides such as raffinose; and polysaccharides such as dextran. Stabilizers may be present in amounts ranging from 0.5 to 10 weight % per weight of antibody.

Non-ionic surfactants or detergents (also known as “wetting agents”) may be added to reduce adsorption to surfaces and to help solubilize the glycoprotein as well as to protect the glycoprotein against agitation-induced aggregation, which also permits the formulation to be exposed to shear surface stress without causing denaturation of the protein. Suitable non-ionic surfactants include polysorbates (20, 80, etc.), poloxamers (184, 188 etc.), and pluronic polyols. Non-ionic surfactants may be present in a range of about 0.05 mg/mL to about 1.0 mg/mL, for example about 0.07 mg/mL to about 0.2 mg/mL.

Additional miscellaneous excipients include bulking agents (e.g., starch), chelating agents (e.g., EDTA), antioxidants (e.g., ascorbic acid, methionine, vitamin E), and cosolvents.

A specific exemplary embodiment of an aqueous composition suitable for administration via intravenous infusion comprises 20 mg/mL anti-SLIT2, 10 mM histidine buffer, pH 6.0, 7% (w/v) sucrose, 0.03% (w/v) polysorbate 80. The composition may be in the form of a lyophilized powder that, upon reconstitution with 5.2 mL sterile water or other solution suitable for injection or infusion (for example, 0.9% saline, Ringer's solution, lactated Ringer's solution, etc.) provides the above aqueous composition. It, or other embodiments of compositions, may also be in the form of a syringe or other device suitable for injection and/or infusion pre-filled with a quantity of composition suitable for a single administration of anti-SLIT2 antibody.

### Methods of Use

ROBO2 is expressed at the basal surface of podocytes; ROBO2-Slit2 signaling has been found to destabilize podocyte focal adhesion. Moreover, glomerular ROBO2 expression has been found to be elevated in membranous nephropathy (MN) and focal segmental glomerulosclerosis (FSGS) patients, indicating the potential overactivation of the pathway (Pisarek-Horowitz et al., Am J Pathol. 2020 April; 190(4):799-816).

Conditional knockout of SLIT2 in podocytes has been found to preserve podocyte foot process after injury and attenuates proteinuria in multiple preclinical disease models (Pisarek-Horowitz et al. 2020). Further, SLIT2 deficiency protects podocyte loss and attenuates proteinuria in multiple preclinical models (Fan et al. 2016; Pisarek-Horowitz et al. 2020). As such, provided herein are methods of utilizing SLIT2 or blocking antibodies for treating glomerular diseases, for example, by maintaining podocyte foot process structure and preventing podocyte loss.

Generally, the methods involve administering to a human patient having cells expressing ROBO2 and/or SLIT2, an amount of an anti-SLIT2 antibody, or a functional fragment or derivative thereof, effective to provide therapeutic benefit. Any method known to one of ordinary skill in the art for assessing the presence and/or expression level of the SLIT2 protein in a cell can be used.

Patients selected for the treatments of this disclosure include those with SLIT2 expressing cells, which include, but are not limited to, kidney tissue expression.

Exemplary therapeutic uses of the anti-SLIT2 antibodies of the present disclosure, including functional fragments or derivatives thereof, include treating a renal disease, such as a glomerular disease, FSGS. The anti-SLIT2 antibodies may also be used in prophylactic treatment (e.g., administering to a subject who has not exhibited a disease symptom but is susceptible to a renal disease such as a glomerular disease, FSGS).

The present disclosure also includes treatment of any disorder, disease or condition mediated by or associated with an increased level of protein in the urine compared with the level of protein in urine in the absence of the disease, disorder or condition. Such disease, disorder or condition includes, but is not limited to, lupus nephritis, IgA nephropathy, membranous nephropathy (MN), minimal change disease (MCD), fibrosis (such as liver fibrosis), nonalcoholic steatohepatitis (NASH), proteinuria, albuminuria, glomerulonephritis, diabetic nephropathy, nephrotic syndrome, focal glomerulosclerosis, acute renal failure, acute tubulointerstitial nephritis, pyelonephritis, renal graft rejection, and reflux nephropathy.

For therapeutic applications, the anti-SLIT2 antibodies of the present disclosure, including functional fragments or derivatives thereof, can be administered to a mammal, especially a human by conventional techniques, such as intravenously (as a bolus or by continuous infusion over a period of time), intramuscularly, intraperitoneally, intra-cerebrospinally, subcutaneously, intra-articularly, intrasynovially, intrathecally, orally, topically, or by inhalation. The antibody, antigen-binding fragment or derivative thereof, of the invention also is suitably administered by intra-tumoral, peri-tumoral, intra-lesional, or peri-lesional routes.

In certain embodiments, the present disclosure provides a method of reducing the activity of ROBO2, comprising administering to a subject (e.g., a human) in need thereof a therapeutically effective amount of an anti-SLIT2 antibody of the present disclosure, including functional fragments or derivatives thereof.

In other embodiments, the present disclosure provides a method of preserving or modulating podocyte function, comprising administering to a subject (e.g., a human) in need thereof a therapeutically effective amount of an anti-SLIT2 antibody of the present disclosure, including functional fragments or derivatives thereof.

In Additional embodiments, the subject suffers from or is susceptible to a renal disease. In certain embodiments, the renal disease is a glomerular disease. In certain embodiments, the renal disease is FSGS. In certain embodiments, the subject suffers from or is susceptible to nephropathy.

In certain embodiments, the antibody, antigen-binding fragment or derivative thereof, of the invention is administered subcutaneously. In certain embodiments, the antibody, antigen-binding fragment or derivative thereof, of the invention is administered intravenously.

The pharmaceutical compositions contemplated herein may be administered to a subject in need thereof at a frequency that may vary with the severity of the renal disease. In the case of prophylactic therapy, the frequency may vary depending on the subject's susceptibility or predisposition to a renal disease.

The compositions may be administered to patients in need, for example, as a bolus or by continuous infusion. For example, a bolus administration of an antibody present as a Fab fragment may be in an amount of from 0.0025 to 100 mg/kg body weight, 0.025 to 0.25 mg/kg, 0.010 to 0.10 mg/kg or 0.10-0.50 mg/kg. For continuous infusion, an antibody present as an Fab fragment may be administered at 0.001 to 100 mg/kg body weight/minute, 0.0125 to 1.25 mg/kg/min, 0.010 to 0.75 mg/kg/min, 0.010 to 1.0 mg/kg/min. or 0.10-0.50 mg/kg/min for a period of 1-24 hours, 1-12 hours, 2-12 hours, 6-12 hours, 2-8 hours, or 1-2 hours.

For administration of an antibody present as a full-length antibody (with constant regions), dosage amounts may be from about 1 mg/kg to about 10 mg/kg, from about 2 mg/kg to about 10 mg/kg, from about 3 mg/kg to about 10 mg/kg, from about 4 mg/kg to about 10 mg/kg, from about 5 mg/kg to about 10 mg/kg, from about 1 mg/kg to about 20 mg/kg, from about 2 mg/kg to about 20 mg/kg, from about 3 mg/kg to about 20 mg/kg, from about 4 mg/kg to about 20 mg/kg, from about 5 mg/kg to about 20 mg/kg, about 1 mg/kg or more, about 2 mg/kg or more, about 3 mg/kg or more, about 4 mg/kg or more, about 5 mg/kg or more, about 6 mg/kg or more, about 7 mg/kg or more, about 8 mg/kg or more, about 9 mg/kg or more, about 10 mg/kg or more, about 11 mg/kg or more, about 12 mg/kg or more, about 13 mg/kg or more, about 14 mg/kg or more, about 15 mg/kg or more, about 16 mg/kg or more, about 17 mg/kg or more, about 19 mg/kg or more, or about 20 mg/kg or more. The frequency of the administration often depends on the severity of the condition. Frequency could range from three times per week to once every two or three weeks.

Additionally, the compositions may be administered to patients via subcutaneous injection. For example, a dose of 1 to 100 mg anti-SLIT2 antibody, including functional fragments or derivatives thereof, can be administered to patients via subcutaneous or intravenous injection administered twice a week, once a week, once every two weeks, once every three weeks, once every four weeks, once every five weeks, once every six weeks, once every seven weeks, once every eight weeks, once every nine weeks, once every ten weeks, twice a month, once a month, once every two months, or once every three months. For example, if the antibody has an estimated half-life of about 19 days with approximately 60% bioavailability following subcutaneous (SC) administration, this half-life supports subcutaneous or intravenous injection at every week, or every 2-6 weeks, such as once every 2 weeks or once every 4 weeks.

In certain embodiments, the half-life of the anti-SLIT2 antibody in a human is about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, from about 5 days to about 40 days, from about 5 days to about 35 days, from about 5 days to about 30 days, from about 5 days to about 25 days, from about 10 days to about 40 days, from about 10 days to about 35 days, from about 10 days to about 30 days, from about 10 days to about 25 days, from about 15 days to about 40 days, from about 15 days to about 35 days, from about 15 days to about 30 days, or from about 15 days to about 25 days.

In certain embodiments, the pharmaceutical composition is administered subcutaneously or intravenously at every 2-6 weeks, with a dose from about 0.1 mg/kg to about 10 mg/kg, from about 0.5 mg/kg to about 10 mg/kg, from about 1 mg/kg to about 10 mg/kg, from about 1.5 mg/kg to about 10 mg/kg, from about 2 mg/kg to about 10 mg/kg, from about 0.1 mg/kg to about 8 mg/kg, from about 0.5 mg/kg to about 8 mg/kg, from about 1 mg/kg to about 8 mg/kg, from about 1.5 mg/kg to about 8 mg/kg, from about 2 mg/kg to about 8 mg/kg, from about 0.1 mg/kg to about 5 mg/kg, from about 0.5 mg/kg to about 5 mg/kg, from about 1 mg/kg to about 5 mg/kg, from about 1.5 mg/kg to about 5 mg/kg, from about 2 mg/kg to about 5 mg/kg, about 0.5 mg/kg, about 1.0 mg/kg, about 1.5 mg/kg, about 2.0 mg/kg, about 2.5 mg/kg, about 3.0 mg/kg, about 3.5 mg/kg, about 4.0 mg/kg, about 4.5 mg/kg, about 5.0 mg/kg, about 5.5 mg/kg, about 6.0 mg/kg, about 6.5 mg/kg, about 7.0 mg/kg, about 7.5 mg/kg, about 8.0 mg/kg, about 8.5 mg/kg, about 9.0 mg/kg, about 9.5 mg/kg, or about 10.0 mg/kg.

In certain embodiments, the pharmaceutical composition is administered subcutaneously or intravenously at every 2-6 weeks, with a dose of about 3.0 mg/kg. In certain embodiments, the pharmaceutical composition is administered subcutaneous or intravenously every 2-6 weeks, with a dose of from about 2.0 mg/kg to about 10.0 mg/kg. For example, in one embodiment, pharmaceutical composition is administered subcutaneously every 2 weeks.

In certain embodiments, the pharmaceutical composition is administered intravenously or intravenously at every 2-6 weeks, with a dose of about 10.0 mg/kg. In certain embodiments, the pharmaceutical composition is administered subcutaneous or intravenously every 2-6 weeks, with a dose of from about 1.0 mg/kg to about 10.0 mg/kg. In one embodiment, pharmaceutical composition is administered intravenously every month.

The anti-SLIT2 antibody of the present disclosure, including functional fragments or derivatives thereof, can be used as monotherapy or in combination with other therapies to treat, e.g., a renal disease. Other therapies for treating real disease are well-known in the art.

Anti-SLIT2 antibodies, including functional fragments or derivatives thereof, may be administered alone (monotherapy) or adjunctive to, or with, other therapies and/or targeted or non-targeted agents. When administered as anti-SLIT2 antibody, binding fragment and/or derivative monotherapy, one or more anti-SLIT2 antibody, binding fragment and/or derivative may be used.

Whether administered as monotherapy or adjunctive to, or with, other therapies or agents, an amount of anti-SLIT2 antibody, binding fragment and/or derivative is administered such that the overall treatment regimen provides therapeutic benefit. By therapeutic benefit is meant that the use of anti-SLIT2 antibody, binding fragment and/or derivative to treat renal disease in a patient results in any demonstrated clinical benefit compared with no therapy (when appropriate) or to a known standard of care. Clinical benefit can be assessed by any method known to one of ordinary skill in the art. In some embodiments, a complete response indicates therapeutic benefit. In some embodiments, a partial response indicates therapeutic benefit. In some embodiments, stable disease indicates therapeutic benefit. In some embodiments, an increase in overall survival indicates therapeutic benefit. In some embodiments, therapeutic benefit may constitute an improvement in time to disease progression and/or an improvement in symptoms or quality of life. In other embodiments, therapeutic benefit may not translate to an increased period of disease control, but rather a markedly reduced symptom burden resulting in improved quality of life. As will be apparent to those of skill in the art, a therapeutic benefit may be observed using the anti-SLIT2 antibody, binding fragment and/or derivative alone (monotherapy) or adjunctive to, or with, other therapies and/or targeted agents.

Typically, therapeutic benefit is assessed using standard clinical tests designed to measure the response to a new treatment for renal disease. To assess the therapeutic benefits of the anti-SLIT2 antibody, binding fragment and/or derivative described herein one or a combination of the following tests can be used: urine protein/creatinine ratio (UPCR), 24 hour urine protein, estimated glomerular filtration rate (eGFR), Blood urea nitrogen (BUN), serum creatinine or another test.

Anti-SLIT2 antibodies, binding fragments and/or derivatives may be used adjunctive to, or with, other agents or treatments. When used adjunctively, the anti-SLIT2 an antibody, binding fragment and/or derivative and other agent(s) may be formulated together in a single pharmaceutical formulation, or may be formulated and administered separately, either on a single coordinated dosing regimen or on different dosing regimens. Agents administered adjunctively with anti-SLIT2 antibody, binding fragment and/or derivative will typically have complementary activities to the anti-SLIT2 antibody, binding fragment and/or derivative and other agents do not adversely affect each other.

Agents that may be used adjunctively with anti-SLIT2 antibody, binding fragment and/or derivative include, but are not limited to Angiotensin Converting Enzyme Inhibitors (ACEI), Angiotensin II Receptor Blockers (ARB), corticosteroids, lipid control therapy, immunosuppressive therapy, anti-coagulation therapy, and/or anti-hypertensive therapy.

Other features and advantages of the invention will be apparent from the following detailed description, and from the claims.

The present invention is further described by the following examples. The examples are provided solely to illustrate the invention by reference to specific embodiments. These exemplifications, while illustrating certain specific aspects of the invention, do not portray the limitations or circumscribe the scope of the disclosed invention.

### EXAMPLES

### Example 1—Antibody Generation in Human Ig Transgenic Mice

Experiments were performed to generate fully human antibodies against human SLIT2 using the following methods.

Human Ig transgenic mice, at 8 weeks of age, were immunized 5 to 6 rounds with human Slit2-his recombinant protein. Antibody titer in the serum was analyzed by human Slit2 indirect ELISA, after each round of immunization, to monitor the increase of Slit2 specific antibody titer. A final boost was given to each mouse 3 days before harvesting the spleen for hybridoma generation.

### SLIT2: O94813(26-1118)-his (SEQ ID NO: 16):

QACPAQCSCSGSTVDCHGLALRSVPRNIPRNTERLDLNGNNITRITKTDFAGLRHLRVLQLMENKISTIERGAFQDLKELE
RLRLNRNHLQLFPELLFLGTAKLYRLDLSENQIQAIPRKAFRGAVDIKNLQLDYNQISCIEDGAFRALRDLEVLTLNNNNIT
RLSVASFNHMPKLRTFRLHSNNLYCDCHLAWLSDWLRQRPRVGLYTQCMGPSHLRGHNVAEVQKREFVCSGHQSFM
APSCSVLHCPAACTCSNNIVDCRGKGLTEIPTNLPETITEIRLEQNTIKVIPPGAFSPYKKLRRIDLSNNQISELAPDAFQGL
RSLNSLVLYGNKITELPKSLFEGLFSLQLLLLNANKINCLRVDAFQDLHNLNLLSLYDNKLQTIAKGTFSPLRAIQTMHLAQ
NPFICDCHLKWLADYLHTNPIETSGARCTSPRRLANKRIGQIKSKKFRCSAKEQYFIPGTEDYRSKLSGDCFADLACPEKCR
CEGTTVDCSNQKLNKIPEHIPQYTAELRLNNNEFTVLEATGIFKKLPQLRKINFSNNKITDIEEGAFEGASGVNEILLTSNRL
ENVQHKMFKGLESLKTLMLRSNRITCVGNDSFIGLSSVRLLSLYDNQITTVAPGAFDTLHSLSTLNLLANPFNQNCYLAW
LGEWLRKKRIVTGNPRCQKPYFLKEIPIQDVAIQDFTCDDGNDDNSCSPLSRCPTECTCLDTVVRCSNKGLKVLPKGIPR
DVTELYLDGNQFTLVPKELSNYKHLTLIDLSNNRISTLSNQSFSNMTQLLTLILSYNRLRCIPPRTFDGLKSLRLLSLHGNDIS
VVPEGAFNDLSALSHLAIGANPLYCDCNMQWLSDWVKSEYKEPGIARCAGPGEMADKLLLTTPSKKFTCQGPVDVNIL
AKCNPCLSNPCKNDGTCNSDPVDFYRCTCPYGFKGQDCDVPIHACISNPCKHGGTCHLKEGEEDGFWCICADGFEGEN
CEVNVDDCEDNDCENNSTCVDGINNYTCLCPPEYTGELCEEKLDFCAQDLNPCQHDSKCILTPKGFKCDCTPGYVGEHC
DIDFDDCQDNKCKNGAHCTDAVNGYTCICPEGYSGLFCEFSPPMVHHHHHH

### Cell Fusion

Cell fusion was done following a standard hybridoma procedure. Briefly, one week before the cell fusion, the fusion partner mouse myeloma cell line sp2/0-Ag14 (ATCC, Cat: CRL-1581) was cultured in complete DMEM medium (2 mM Glutamine and 10% Fetal Bovine Serum, FBS). Spleens from the immunized mice were mechanically processed and made into single-cell suspensions. The cell suspension was passed through a fine mesh nylon filter and transferred into a sterile 50 mL centrifuge tube full of complete DMEM medium.

The splenocytes were pelleted by centrifuging for 5 minutes at 200×g at room temperature. Supernatant was discarded and the cell pellet was resuspended with 1 mL of red blood cell lysis buffer (Sigma, Cat: R7757) for 1 minute at room temperature to remove red blood cells. Cells were then washed twice with complete DMEM medium and then counted using a hemocytometer.

Concurrently, sp2/0-Ag14 myeloma cells were harvested by transferring the cells from their culture vessels into a 50 mL centrifuging tube and washed twice with complete DMEM medium followed by cell count and viability assessment.

The myeloma cells and the mouse splenocytes were mixed at a 1:4 ratio, respectively, in a 50 mL centrifuge tube and then spun down for 5 minutes at 200×g at room temperature. The cell fusions were performed by electrical pulse (NEPAGENE, ECFG21). The fused cells were then thoroughly resuspended with 13 mL of pre-warmed complete DMEM medium and then incubated in a humidified incubator at 37° C. with 5% CO₂ for 10 minutes.

After then, the fused cells were transferred to 96-well flat bottom plates until the entire cell suspension was plated with complete HAT medium (complete DMEM medium supplemented with 1×HAT (sigma, Cat: H0262) (5*10⁴ splenocytes/well). The fused cells were kept in a humidified incubator at 37° C. with 5% CO₂. On culture 3 days, the fused cells were fed with new complete HAT media. On culture 6 days, the fused cells were fed by removing 50% of the original culture media and replacing it with new complete HAT media. On culture 8 days, the fused cells were fed by removing all the original culture media and replacing them with new complete HT (complete DMEM medium supplemented with 1×HT (sigma, Cat: H0260) media.

After 10 days in culture, fusion supernatants were evaluated for anti-Slit2 activity using high-throughput binding and blocking ELISA assays.

Cloning VH and VL Sequences from Hybridomas

For determination of antibody heavy and light chain sequences, total RNA was isolated from hybridoma cells using an UNIQ-10 Column Total RNA Purification Kit (Sangon Biotech Cat: B511361). First and second-strand cDNA synthesis was performed using a HiScript II 1st Strand cDNA Synthesis Kit (Vazyme Cat: R211-02). Several primer sets were used (human HVs-F and mouse HC-R, human KVs-F and mouse KC-R). PCR products were analyzed by agarose electrophoresis before sending the samples to Sangon for DNA sequencing. Sequencing results were analyzed by aligning sequence to the database from IMGT. Both heavy and light chain sequences were then chemically synthesized and cloned into expression vectors by seemless cloning techniques. 2 or 3 colonies from each hybridoma cell were scaled up for miniprep-scale plasmid purification by SanPrep Column Plasmid Mini-Preps DNA Purification Kit (Sangon Biotech Cat: B518191).

### Identification of Functional, Recombinant VH and VL Sequences

Antibody expression vectors were transfected into Expi293 cells in 20 ml production scale. After five days, conditioned medium from each pairing was subjected to concentration determination by Modified BCA Protein Assay Kit (Sangon biotech, Cat: C503051) and screened by ELISA or flow cytometry assay for binding and blocking activity.

### Transient Expression System

Anti-Slit2 antibodies were expressed in expi293 cells using recommended transfection. Cell culture supernatants were harvested five days post-transfection, centrifuged, and filtered (0.22 um).

### Antibody Purification

Conditioned medium from expi293 cell cultures was clarified, filtered, and purified with rProteinG beads (Smart-lifesciences, Cat: SA016005). Antibodies were eluted with 100 mM glycine, pH 2.5 and neutralized with 1 M Tris-Cl, pH 8.5.

### Example 2—ELISA Binding of Anti-Slit2 Antibodies

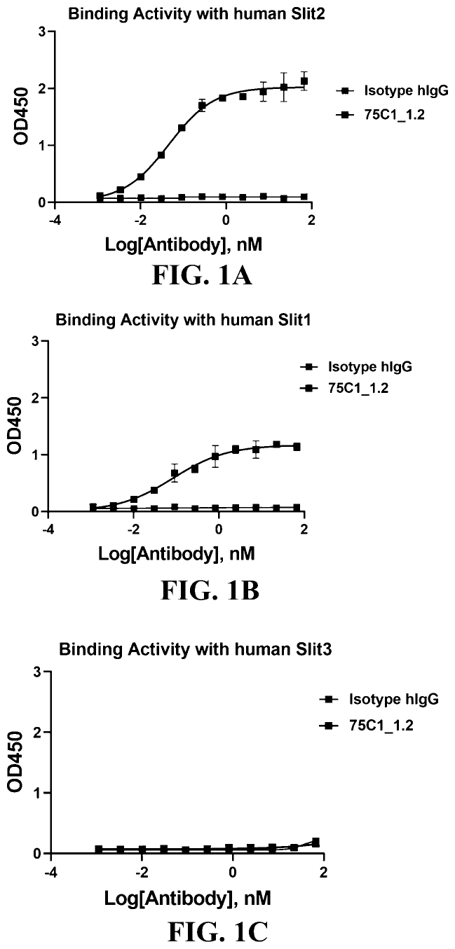

Binding of anti-Slit2 antibodies to human Slit1, Slit2 and Slit3 was measured by ELISA. Antibodies were diluted into phosphate buffered saline (PBS) (1×) to give 1 μg/ml solution. 100 μL of the diluted stocks was then added to 96 well Elisa plates. The Slit1, Slit2 and Slit3 antigens were allowed to absorb to the plates overnight at 4° C., washed once with 1×PBS and incubated with blocking buffer at room temperature for 2 hours. The plates were then incubated for 1 hour with serial diluted Slit proteins from 2 μg/ml diluted in 1×PBS containing 1% Bovine Serum Albumin (BSA). The plates were then washed three times with 1×PBS containing 0.05% Tween (1×PBST) and incubated with peroxidase conjugated anti-his antibody (Biolegend) for 1 hour at room temperature. The plates were then washed three times with 1×PBST. Following a 5-minute incubation with TMB peroxidase substrate, the reaction was stopped by the addition of 0.5 M H₂SO₄. Absorbance at 450 nm (A450) was measured using an Envision multilabel plate reader (Perkin Elmer, Seer Green, UK). (FIG. 1A-C, TABLE 13)

TABLE 13
EC50 Values for anti-SLIT2 bind with 2 other Slit family members

| | HUMAN SLIT2 | HUMAN SLIT1 | HUMAN SLIT3 |
|---|---|---|---|
| Antibody | (nM) | (nM) | (nM) |
| 75C1_1.2 | 0.046 | 0.084 | No binding |

EC50 or the concentration of antibody that gives half-maximal binding is determined by direct and saturable binding of to both target antigen and a non-target control antigens. An estimate of affinity is interpreted from one-half the concentration at which binding first achieves saturation. These results show strong affinity of the anti-Slit2 antibody mutant 75C1_1.2 for the human SLIT2 antigen in addition to the SLIT1 antigen, with no discernable binding of the Slit3 antigen.

### Example 3—BLI Binding Data of Anti-Slit2 Antibodies

Anti-Slit2 antibody, mutants described herein, affinity measurements were performed on a GatorPrime generally as previously. Briefly, affinity measurements were performed by loading IgGs on-line onto HFC probes. Probes were equilibrated off-line in assay buffer for 30 minutes and then monitored on-line for 120 seconds for baseline establishment. Sensors with loaded IgGs were exposed to 100 nM Slit2-his antigen (full length, human) for 300 seconds, and afterwards were transferred to assay buffer for 300 seconds for off-rate measurement. All kinetics were analyzed using the 1:1 binding model (TABLE 14).

TABLE 14

| Ligand | KD (M) | Kₒₙ(1/Ms) | K_{dis}(1/s) | Full R² |
|---|---|---|---|---|
| 75C1 | 4.02E−10 | 1.45E+05 | 5.82E−05 | 0.9802 |
| 75C1_1.1 | 4.03E−10 | 1.35E+05 | 5.44E−05 | 0.9832 |
| 75C1_1.2 | 4.08E−10 | 1.37E+05 | 5.59E−05 | 0.9822 |
| 75C1_1.3 | 4.09E−10 | 1.45E+05 | 5.94E−05 | 0.9816 |
| 75C1_1.4 | 4.14E−10 | 1.40E+05 | 5.78E−05 | 0.9816 |
| 75C1_1.5 | 4.19E−10 | 1.46E+05 | 6.14E−05 | 0.9809 |
| 75C1_1.6 | 4.18E−10 | 1.28E+05 | 5.37E−05 | 0.9845 |
| 75C1_1.7 | 3.92E−10 | 1.39E+05 | 5.47E−05 | 0.9836 |
| 75C1_1.8 | 4.09E−10 | 1.26E+05 | 5.14E−05 | 0.9836 |
| 75C1_2.1 | 4.04E−10 | 1.28E+05 | 5.18E−05 | 0.9848 |
| 75C1_2.2 | 4.49E−10 | 1.38E+05 | 6.18E−05 | 0.9824 |
| 75C1_2.3 | 4.49E−10 | 1.33E+05 | 5.98E−05 | 0.9843 |
| 75C1_2.4 | 4.40E−10 | 1.09E+05 | 4.78E−05 | 0.9871 |
| 75C1_2.5 | 3.66E−10 | 1.35E+05 | 4.96E−05 | 0.9842 |
| 75C1_2.6 | 4.00E−10 | 1.24E+05 | 4.98E−05 | 0.9844 |
| 75C1_2.7 | 4.44E−10 | 1.21E+05 | 5.37E−05 | 0.9854 |
| 75C1_2.8 | 4.26E−10 | 1.29E+05 | 5.51E−05 | 0.9834 |

KD is the equilibrium dissociation constant, a ratio of koff/kon, between the antibody and its antigen. KID and affinity are inversely related. The KID value relates to the concentration of antibody, and so the lower the KID value, the higher the affinity of the antibody. Association Reaction (Kon) is used to calculate the “on-rate” (Kon), a constant used to characterize how quickly the antibody binds to its target. Dissociation Reaction (Koff) is used to calculate the “off-rate” (Koff), a constant used to characterize how quickly an antibody dissociates from its target. As is shown, various of the 75C1 mutants have comparable KID values to the 75C1 anti-Slit2 antibody.

### Example 4—Species Cross-Reactivity of Anti-Slit2 Antibodies

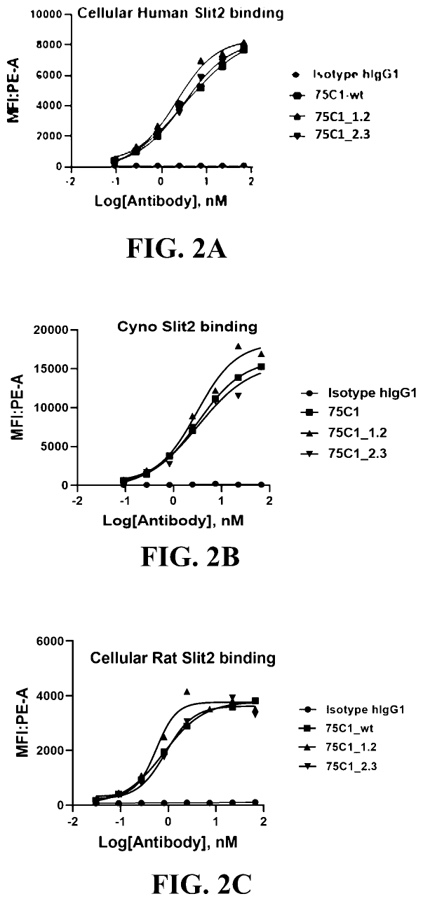

The ability of anti-Slit2 monoclonal antibodies to bind to human, cyno and rat Slit2 expressing cells was determined by flow cytometry assay (FACS). Briefly, Human embryonic kidney 293 (HEK293) were transfected human, cyno or rat Slit2 plasmids, respectively. Cells lines from described above were rinsed once in PBS buffer without Ca2+^{/}Mg2+ and incubated for 2^{˜}3 minutes at 37° C. with Trypsin. All cells were washed once with PBS buffer and counted with a NucleoCounterRNC-200TMAuto cell counter (Chemometec). Approximately 1.0×10⁵ cells were seeded separately onto 96-well round bottom plates. Serial dilutions of anti-Slit2 or isotype control antibodies, ranging from 91.45 pM to 66.67 nM, or buffer containing no antibody were added to the plate for 1 hour, 4° C. Plates were then washed to remove unbound antibodies. The plate-bound antibodies were detected with Goat anti-human IgG-PE antibody specific for heavy and light chains (Invitrogen, 12-4998-82) for 1 hour, 4° C. After washing, cells were resuspended in ice cold PBS for data acquisition on BD FACS. Data was analyzed using FlowJo software and represented as medium fluorescence intensity (MFI) (TABLE 15; FIG. 2A, 2B, 2C).

TABLE 15
EC50 Values for anti-SLIT2 bind with Slit2 of different species

| | HUMAN SLIT2 | CYNO SLIT2 | RAT SLIT2 |
|---|---|---|---|
| Antibody | (nM) | (nM) | (nM) |
| 75C1 | 3.21 | 3.20 | 0.81 |
| 75C1_1.2 | 2.22 | 3.05 | 0.56 |
| 75C1_2.3 | 3.09 | 3.24 | 0.89 |

This compares binding activity of two anti-Slit2 antibody (75C1) mutants with 75C1 anti-Slit2 in binding human versus cyno and rat Slit2. Slit2 is relatively highly conserved among varying species. Each had a similar range of affinity relative to the cyno and rat Slit antigens, with the anti-Slit2 mutant 75C1_1.2 demonstrating slightly higher affinity for each.

### Example 5—Blocking Data of Anti-Slit2 Antibodies

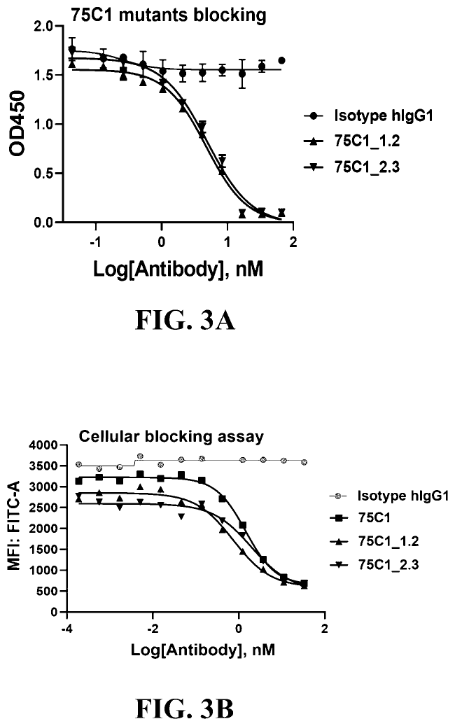

Blocking activity of the anti-Slit2 antibodies to Robo2-Slit2 interaction was measured by ELISA. Recombinant human Robo2 ECD proteins were diluted into 1×PBS to give 2 μg/ml solution. 50 μL of the diluted stocks was then added to 96 well Elisa plates. The antigens were allowed to absorb to the plates overnight at 4° C., washed once with 1×PBS and incubated with blocking buffer at 37° C. for 2 hours. 50 μL of serial diluted anti-Slit2 (starting from 8 μg/ml) was pre-incubated with 50 μL human Slit2 D2 domain-His (final conc at 0.5 ug/ml) at 37 degree for 30 minutes before adding to the plate for another 30 minutes. The plates were then washed three times with 1×PBST and incubated with HRP anti-His antibody (Biolegend) for 1 hour at room temperature. The plates were then washed three times with 1×PBST. Following a 5-minute incubation with TMB, the reaction was stopped by 0.5 M H₂SO₄. A450 was measured using an Envision multilabel plate reader (Perkin Elmer, Seer Green, UK). Blocking activity of the anti-Slit2 antibodies to Robo2-Slit2 interaction was also measured by FACS at cellular level. Firstly, the hROBO2-ECD hFc protein was labeled with Alexa Fluor 488 fluorescent signal using the Alexa Fluor Antibody Labeling Kit (Invitrogen, A20181). After successful labeling of proteins, antibody blocking robo2-slit2 interaction was tested in human Slit2 over-expressing HEK293 cells. In brief, cells were digested with trypsin and counted using a cell counter. About 1.0×10⁵ cells were seeded separately onto 96-well round bottom plates. Serial dilutions of anti-Slit2 or isotype control antibodies, ranging from 91.45 pM to 66.67 nM, or buffer containing no antibody were added to plate-bound cells for 30 minutes, 4° C. Plates were then added hROBO2-Alexa Fluor 488 solution for 1.5 hours, 4° C. FACS plates were then washed to remove unbound antibodies. After washing, cells were resuspended in ice cold PBS for data acquisition on BD FACS. Data was analyzed using FlowJo software and represented as MFI (TABLES 16 and 17 and FIGS. 3A and 3B)

TABLE 16
IC50 Values for anti-SLIT2 antibody
Against SLIT2-ROBO2 Interaction

| Antibody | IC₅₀ (nM) |
|---|---|
| 75C1_1.2 | 4.72 |
| 75C1_2.3 | 4.88 |

These results indicate potentblocking activity of the above two anti-Slit2 antibodies.

TABLE 17

| Antibody | IC50 (nM) |
|---|---|
| 75C1 | 1.45 |
| 75C1_1.2 | 0.74 |
| 75C1_2.3 | 1.95 |

In this experiment the anti-Slit2 mutant 75C1_1.2 is shown to have higher blocking potency than the anti-Slit2 antibody 75C1.

### Example 6

SLIT2-Robo2 signaling pathway has been shown to inhibit neuronal cell migration. The functional neutralization activity of antibodies was evaluated by SLIT2-induced migration inhibition on ROBO2-HEK293 cells.

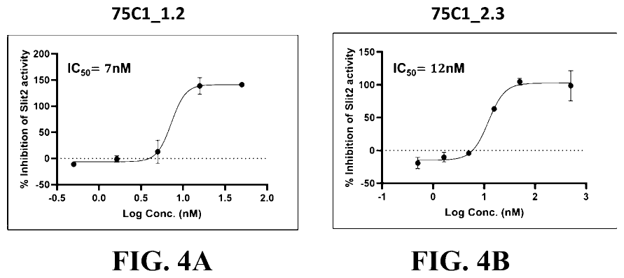

Human embryonic kidney 293 cells overexpressing human ROBO2 (ROBO2-HEK293) were starved in serum-free medium for 4 h. Starved cells were collected and seeded to the upper chamber of the RTCA CIM-plate16. Slit-N and blocking antibodies were added to the basolateral wells of the RTCA CIM-plate16. Cell migration was measured by RTCA xCELLigence DP system (FIGS. 4A and 4B). As is shown in FIGS. 4A and 4B, the anti-Slit2 antibody blocked Slit2 activity in a dose-dependent manner. The IC₅₀ is 7 nM and 12 nM for 75C1_1.2 and 75C1_2.3, respectively.

### Example 7

Passive Heymann Nephritis (PHN) model is a commonly used model to study podocyte response to injury. After receiving sheep anti-rat Fx1A serum, rats develop podocyte effacement and proteinuria due to immune complex deposition and complement activation. The PHN model closely resembles the features of human disease Membranous Nephropathy which also involves antibody-mediated glomerular injury and podocytopathy (PMID: 21359154).

Lewis rats were injected i.v. with 2.5 ml/kg anti-rat Fx1A serum (Probetex Inc) on Day 0. Testing antibodies were injected i.v. on Day −1, 2, 5, 8, 11 and 14. Urine samples were collected on Day −1, 7, 10, 14 and 17 for albumin, creatine, and total protein measurement. Rats were euthanized on Day 17 and kidney samples were collected for pathology studies.

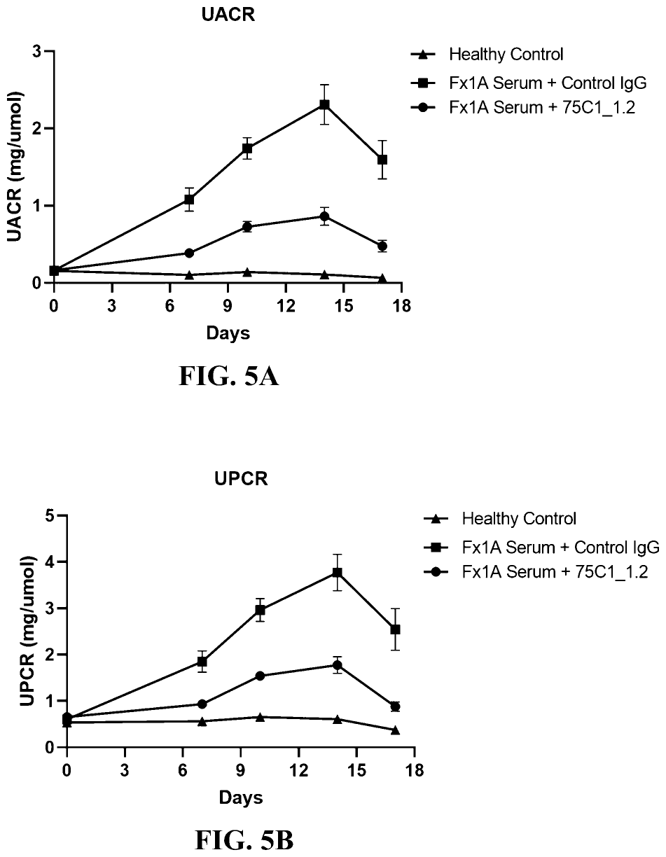

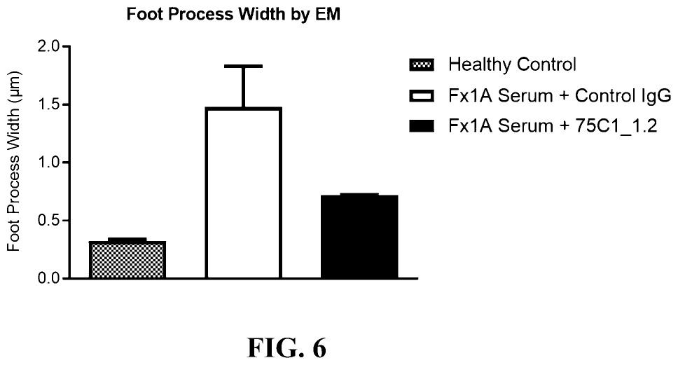

Treatment with anti-Slit2 antibodies significantly reduced the urine albumin-creatine ratio (UACR) and urine albumin-protein ratio (UPCR). The maximal reduction in proteinuria was 52% (FIGS. 5A and 5B). To further confirm the mechanism on podocyte structure modulation, analysis of podocyte foot process width was performed by electron microscope. As shown in FIG. 6, treatment with anti-Slit2 antibodies significantly reduced foot process width, which indicates the treatment protected podocyte from effacement.

### Example 8—Pharmacokinetics

### I. Methods of Analysis—Quantitation of 75C1_1.2 In Vivo

An Enzyme Linked Immunosorbent Assay (ELISA) assay is validated for the quantitation of 75C1_1.2 in subjects (e.g., Sprague Dawley (SD) rat, Cynomolgus monkey, etc.). In these assays, samples containing 75C1_1.2 are incubated onto 96 well microplates coated with goat anti Human IgG antibody. Detection is achieved by adding recombinant human (HEK293-derived) solution to produce absorbance signal that is read at 450-630 nm with a plate reader, which is proportional to the concentration of 75C1_1.2. Sample concentrations are determined by interpolation from a standard curve that is fit using a 4-parameter logistic (auto-estimate) model with a weighting factor of 1/values\hat{ }2. The range of quantitation is determined (e.g., with a minimum required dilution factor of 1:50).

### II. Pharmacokinetics

### A. Single-Dose Pharmacokinetics

The serum PK of 75C1_1.2 is determined in subjects (e.g., Sprague Dawley rats, cynomolgus monkeys, etc.) following a single IV dose at 10 mg/kg. Clearance of 75C1_1.2 is exhibited resulting in half-life determinations in subjects. The serum maximum concentration (Cmax) in subjects is determined. The area under the concentration curve (AUC0-last) is also determined. Following 10 mg/kg SC administration of 75C1_1.2 to subjects, the Cmax and AUC0-last is determined as well as an estimated bioavailability.

### B. Repeat-Dose Pharmacokinetics

Multiple dose PK evaluations are conducted after IV administration of 10 mg/kg/dose, given once every week and totally 4 doses to subjects. The maximum concentration (Cmax) post first and last dose is determined. The area under the concentration curve from time 0 to 168 hours (AUC0-168 h) post first and last dose are also determined. The accumulation ratios of Cmax and AUC0-168 h are identified.

### III. Distribution

Steady state volume of distribution (Vdss) is determined in subjects according to known means.

### Embodiments

According to embodiment 1, an antibody is provided comprising: a heavy chain comprising complementarity determining regions (CDR) CDR-H1 comprising amino acid sequence of SEQ ID NO: 1; CDR-H2 comprising amino acid sequence of SEQ ID NO: 2; and CDR-H3 comprising amino acid sequence of SEQ ID NO: 3, respectively and having a specific binding activity to human SLIT2 and/or blocking activity to ROBO2; and a light chain comprising CDR-L1 comprising amino acid sequence of SEQ ID NO: 4 or 7; CDR-L2 comprising amino acid sequence of SEQ ID NO: 5; and CDR-L3 comprising amino acid sequence of SEQ ID NO: 6 and having a specific binding activity to human SLIT2 and/or blocking activity to ROBO2.

According to embodiment 2, an antibody of embodiment 1 is provided wherein the antibody is a chimeric antibody.

According to embodiment 3, an antibody of embodiments 1 or 2 is provided, wherein the antibody is a human antibody.

According to embodiment 4, an antibody of any of embodiments 1-3 is provided, wherein the antibody is a humanized antibody.

According to embodiment 5, an antibody of any of embodiments 1-4 is provided, wherein the antibody comprises: a heavy chain variable domain comprising an amino acid sequence at least 85% identical to any of the amino acid sequence of SEQ ID NOS: 8, 24-27, optionally wherein the heavy chain variable domain includes CDR-H1, CDR-H2 and CDR-H3 defined by SEQ ID NOS:1-3, respectively; and a) a light chain variable domain comprising an amino acid sequence at least 85% identical to any of the amino acid sequence of SEQ ID NOS: 9, 28-29, wherein the light chain variable domain includes CDR-L1, CDR-L2 and CDR-L3 defined by SEQ ID NOS:4-6, respectively, or b) a light chain variable domain comprising an amino acid sequence at least 85% identical to any of the amino acid sequence of SEQ ID NOS: 30-31, optionally wherein the light chain variable domain includes CDR-L1, CDR-L2 and CDR-L3 defined by SEQ ID NOS:7, 5 and 6, respectively.

According to embodiment 6, an antibody of any of embodiments 1-4 is provided, wherein the antibody comprises: a heavy chain variable domain comprising an amino acid sequence at least 90% identical to any of the amino acid sequence of SEQ ID NOS: 8, 24-27, optionally wherein the heavy chain variable domain includes CDR-H1, CDR-H2 and CDR-H3 defined by SEQ ID NOS:1-3, respectively; and a) a light chain variable domain comprising an amino acid sequence at least 90% identical to any of the amino acid sequence of SEQ ID NOS: 9, 28-29, optionally wherein the light chain variable domain includes CDR-L1, CDR-L2 and CDR-L3 defined by SEQ ID NOS:4-6, respectively, or b) a light chain variable domain comprising an amino acid sequence at least 90% identical to any of the amino acid sequence of SEQ ID NOS: 30-31, optionally wherein the light chain variable domain includes CDR-L1, CDR-L2 and CDR-L3 defined by SEQ ID NOS:7, 5 and 6, respectively.

According to embodiment 7, an antibody of any of embodiments 1-4 is provided, wherein the antibody comprises: a heavy chain variable domain comprising an amino acid sequence at least 95% identical to any of the amino acid sequence of SEQ ID NOS: 8, 24-27, optionally wherein the heavy chain variable domain includes CDR-H1, CDR-H2 and CDR-H3 defined by SEQ ID NOS:1-3, respectively; and a) a light chain variable domain comprising an amino acid sequence at least 95% identical to any of the amino acid sequence of SEQ ID NOS: 9, 28-29, optionally wherein the light chain variable domain includes CDR-L1, CDR-L2 and CDR-L3 defined by SEQ ID NOS:4-6, respectively, or b) a light chain variable domain comprising an amino acid sequence at least 95% identical to any of the amino acid sequence of SEQ ID NOS: 30-31, optionally wherein the light chain variable domain includes CDR-L1, CDR-L2 and CDR-L3 defined by SEQ ID NOS:7, 5 and 6, respectively.

According to embodiment 8, an antibody of any of embodiments 5-7 is provided, wherein the antibody comprises a human light chain constant region and a human heavy chain constant region.

According to embodiment 9, an antibody of embodiment 8 is provided, wherein the human light chain constant region is of the IgG1 kappa isotype.

According to embodiment 10, an antibody of any of embodiments 1-9 is provided, wherein the antibody comprises a human light chain constant region and a human heavy chain constant region.

According to embodiment 11, an antibody of embodiment 10 is provided, wherein the human light chain constant region is of the IgG1 kappa isotype.

According to embodiment 12, a pharmaceutical composition is provided comprising the antibody according to any one of embodiments 1-11 and a pharmaceutically acceptable vehicle.

According to embodiment 13, a recombinant antibody is provided comprising: a heavy chain variable domain comprising the amino acid sequence of any of the amino acid sequence of SEQ ID NOS: 8, 24-27; and a light chain variable domain comprising the amino acid sequence of any of the amino acid sequence of SEQ ID NOS: 9, 28-31.

According to embodiment 14, an antibody of embodiment 13 is provided, wherein the antibody is a chimeric antibody.

According to embodiment 15, an antibody of embodiment 13 is provided, wherein the antibody is a human antibody.

According to embodiment 16, an antibody of embodiment 13 is provided wherein the antibody is a humanized antibody.

According to embodiment 17, a pharmaceutical composition is provided comprising the antibody according to any one of embodiment 13 to embodiment 16 and a pharmaceutically acceptable vehicle.

According to embodiment 18, a recombinant antibody capable of modulating ROBO2 and SLIT2 interaction is provided, wherein the antibody comprises: a heavy chain comprising an amino acid sequence at least 85% identical to any of the amino acid sequences of SEQ ID NOS: 12, 44-47 and having a specific binding activity to human SLIT2 and/or blocking activity to ROBO2, optionally wherein the heavy chain includes CDR-H1, CDR-H2 and CDR-H3 defined by SEQ ID NOS:1-3, respectively; and a) a light chain comprising an amino acid sequence at least 85% identical to any of the amino acid sequences of SEQ ID NO: 13, 48-49 and having a specific binding activity to human SLIT2 and/or blocking activity to ROBO2, optionally wherein the light chain variable domain includes CDR-L1, CDR-L2 and CDR-L3 defined by SEQ ID NOS:4-6, respectively, or b) a light chain comprising an amino acid sequence at least 85% identical to any of the amino acid sequences of SEQ ID NOS: 50-51 and having a specific binding activity to human SLIT2 and/or blocking activity to ROBO2, optionally wherein the light chain variable domain includes CDR-L1, CDR-L2 and CDR-L3 defined by SEQ ID NOS:7, 5 and 6, respectively.

According to embodiment 19, a recombinant antibody capable of modulating ROBO2 and SLIT2 interaction is provided, wherein the antibody comprises: a heavy chain comprising an amino acid sequence at least 90% identical to any of the amino acid sequences of SEQ ID NOS: 12, 44-47 and having a specific binding activity to human SLIT2 and/or blocking activity to ROBO2, optionally wherein the heavy chain includes CDR-H1, CDR-H2 and CDR-H3 defined by SEQ ID NOS:1-3, respectively; and a) a light chain comprising an amino acid sequence at least 90% identical to any of the amino acid sequences of SEQ ID NO: 13, 48-49 and having a specific binding activity to human SLIT2 and/or blocking activity to ROBO2, optionally wherein the light chain variable domain includes CDR-L1, CDR-L2 and CDR-L3 defined by SEQ ID NOS:4-6, respectively, or b) a light chain comprising an amino acid sequence at least 90% identical to any of the amino acid sequences of SEQ ID NOS: 50-51 and having a specific binding activity to human SLIT2 and/or blocking activity to ROBO2, optionally wherein the light chain variable domain includes CDR-L1, CDR-L2 and CDR-L3 defined by SEQ ID NOS:7, 5 and 6, respectively.

According to embodiment 20, a recombinant antibody capable of modulating ROBO2 and SLIT2 interaction is provided, wherein the antibody comprises: a heavy chain comprising an amino acid sequence at least 95% identical to any of the amino acid sequences of SEQ ID NOS: 12, 44-47 and having a specific binding activity to human SLIT2 and/or blocking activity to ROBO2, optionally wherein the heavy chain includes CDR-H1, CDR-H2 and CDR-H3 defined by SEQ ID NOS:1-3, respectively; and a) a light chain comprising an amino acid sequence at least 95% identical to any of the amino acid sequences of SEQ ID NO: 13, 48-49 and having a specific binding activity to human SLIT2 and/or blocking activity to ROBO2, optionally wherein the light chain variable domain includes CDR-L1, CDR-L2 and CDR-L3 defined by SEQ ID NOS:4-6, respectively, or b) a light chain comprising an amino acid sequence at least 95% identical to any of the amino acid sequences of SEQ ID NOS: 50-51 and having a specific binding activity to human SLIT2 and/or blocking activity to ROBO2, optionally wherein the light chain variable domain includes CDR-L1, CDR-L2 and CDR-L3 defined by SEQ ID NOS:7, 5 and 6, respectively.

According to embodiment 21, a recombinant antibody capable of modulating ROBO2 and SLIT2 interaction is provided, wherein the antibody comprises: a heavy chain comprising the amino acid sequence of any of the amino acid sequences of SEQ ID NOS: 12, 44-47; and a light chain comprising the amino acid sequence of any of the amino acid sequences of SEQ ID NO: 13, 48-51.

According to embodiment 22, a pharmaceutical composition is provided comprising the antibody according to any one of embodiment 18 to embodiment 21 and a pharmaceutically acceptable vehicle.

According to embodiment 23, an antibody of any of embodiments 1-11, 13-16, 18-21 is provided, comprising an Fc domain, wherein the Fc domain is the Fc domain of an IgA1 IgA2, IgD, IgE, IgM, IgG1, IgG2, IgG3, or IgG4.

According to embodiment 24, an antibody that competes for binding to SLIT2 with the antibody of any of embodiments 1-11, 13-16, 18-21 or 23 is provided.

According to embodiment 25, an isolated nucleic acid molecule is provided, comprising one or more nucleotide sequences encoding the antibody of any of embodiments 1-11, 13-16, 18-21 or 23-24.

According to embodiment 26, a vector is provided comprising the nucleic acid molecule of embodiment 25.

According to embodiment 27, a host cell is provided comprising the nucleic acid molecule of embodiment 25.

According to embodiment 28, a host cell is provided comprising the vector of embodiment 26.

According to embodiment 29, a method of making the antibody of any one of embodiments 1-11, 13-16, 18-21 or 23-24 is provided, comprising expressing the antibody in a host cell and isolating the antibody from the host cell.

According to embodiment 30, a method of treating a renal disease is provided, comprising administering to a subject in need thereof a therapeutically effective amount of the antibody of any of embodiment 1-11, 13-16, 18-21 or 23-24, or the pharmaceutical composition of embodiment 12, 17 or 22.

According to embodiment 31, the method of embodiment 30 is provided, wherein the renal disease is a glomerular disease, Focal Segmental Glomerular Sclerosis (FSGS), or nephropathy.

According to embodiment 32, a method of reducing proteinuria is provided, comprising administering to a subject in need thereof a therapeutically effective amount of the antibody of any of embodiment 1-11, 13-16, 18-21 or 23-24, or the pharmaceutical composition of embodiment 12, 17 or 22.

According to embodiment 33, the method of embodiment 32 is provided, wherein the subject is afflicted with a renal disease, wherein said renal disease is a glomerular disease, Focal Segmental Glomerular Sclerosis (FSGS), or nephropathy.

According to embodiment 34, the method of embodiment 32 is provided, wherein said subject is susceptible to a renal disease, wherein said renal disease is a glomerular disease, Focal Segmental Glomerular Sclerosis (FSGS), or nephropathy.

According to embodiment 35, a method of preserving podocyte function is provided, comprising administering to a subject in need thereof a therapeutically effective amount of the antibody of any of embodiment 1-11, 13-16, 18-21 or 23-24, or the pharmaceutical composition of embodiment 12, 17 or 22.

According to embodiment 36, the method of embodiment 35 is provided, wherein the subject is afflicted with a renal disease, wherein said renal disease is a glomerular disease, Focal Segmental Glomerular Sclerosis (FSGS), or nephropathy.

According to embodiment 37, the method of embodiment 35 is provided, wherein the subject is susceptible to a renal disease, wherein said renal disease is a glomerular disease, Focal Segmental Glomerular Sclerosis (FSGS), or nephropathy.

According to embodiment 38, an isolated nucleic acid molecule encoding variable regions of an antibody is provided, comprising: a nucleotide sequence encoding a heavy chain variable region of SEQ ID NOS: 10, 32-35; and a nucleotide sequence encoding a light chain variable region of SEQ ID NOS: 11, 36-39.

According to embodiment 39, an isolated nucleic acid molecule encoding antibody heavy and light chains is provided, comprising: a nucleotide sequence encoding a heavy chain of SEQ ID NOS: 14, 52-55; and a nucleotide sequence encoding a light chain of SEQ ID NOS: 15, 56-59.

According to embodiment 40, a recombinant antibody heavy chain or fragment thereof having specific binding activity to human SLIT2 and/or blocking activity to ROBO2 is provided, wherein the recombinant antibody heavy chain or fragment thereof comprises the amino acid sequence of SEQ ID NO: 1, 2 and/or 3.

According to embodiment 41, a recombinant antibody light chain or fragment thereof having specific binding activity to human SLIT2 and/or blocking activity to ROBO2 is provided, wherein the recombinant antibody light chain or fragment thereof comprises the amino acid sequence of SEQ ID NO: 4, 5, 6 and/or 7.

According to embodiment 42, a recombinant antibody heavy chain variable region having specific binding activity to human SLIT2 and/or blocking activity to ROBO2 is provided, wherein the recombinant antibody heavy chain variable region comprises the amino acid sequence of any one of SEQ ID NOS: 8, 24-27.

According to embodiment 43, a recombinant antibody light chain variable region having specific binding activity to human SLIT2 and/or blocking activity to ROBO2 is provided, wherein the recombinant antibody light chain variable region comprises the amino acid sequence of any one of SEQ ID NOS: 9, 28-31.

According to embodiment 44, a recombinant antibody heavy chain having specific binding activity to human SLIT2 and/or blocking activity to ROBO2 is provided, wherein the recombinant antibody heavy chain comprises the amino acid sequence of any one of SEQ ID NOS: 12, 44-47.

According to embodiment 45, a recombinant antibody light chain having specific binding activity to human SLIT2 and/or blocking activity to ROBO2 is provided, wherein the recombinant antibody light chain comprises the amino acid sequence of any one of SEQ ID NOS: 13, 48-51.

According to embodiment 46, an antibody is provided comprising: a heavy chain variable domain comprising the amino acid sequence of SEQ ID NO: 25; and a light chain variable domain comprising the amino acid sequence of SEQ ID NO: 28.

According to embodiment 47, an antibody is provided comprising: a heavy chain variable domain comprising the amino acid sequence of SEQ ID NO: 26; and a light chain variable domain comprising the amino acid sequence of SEQ ID NO: 30.

According to embodiment 48, an antibody is provided comprising: a heavy chain comprising the amino acid sequence of SEQ ID NO: 45; and a light chain comprising the amino acid sequence of SEQ ID NO: 48.

According to embodiment 49, an antibody is provided comprising: a heavy chain comprising the amino acid sequence of SEQ ID NO: 46; and a light chain comprising the amino acid sequence of SEQ ID NO: 50.

According to embodiment 50, the antibody of any of embodiments 46-49 is provided, wherein the antibody is a chimeric antibody.

According to embodiment 51, the antibody of any of embodiments 46-49 is provided, wherein the antibody is a humanized antibody.

According to embodiment 52, the antibody of any of embodiments 46-49 is provided, wherein the antibody is a human antibody.

According to embodiment 53, the antibody of any of embodiments 46-51 is provided, wherein the antibody comprises a human light chain constant region and a human heavy chain constant region.

According to embodiment 54, the antibody of embodiment 52 or 53 is provided, wherein the human light chain constant region is of the IgG1 kappa isotype.

According to embodiment 55, the antibody of any of embodiments 46-54 is provided, wherein the antibody has a specific binding activity to human SLIT2 and/or blocking activity to ROBO2.

According to embodiment 56, the antibody of any of embodiments 46-55 is provided, wherein the antibody is a recombinant antibody.

According to embodiment 57, the antibody of any of embodiments 46-56 is provided, wherein the antibody is capable of modulating ROBO2 and SLIT2 interaction.

According to embodiment 58, the antibody of any of embodiments 55-57 is provided, wherein at least a portion of the SLIT2 has the amino acid sequence of SEQ ID NO: 16.

According to embodiment 59, the antibody of any of embodiments 46-58 is provided, comprising an Fc domain, wherein the Fc domain is the Fc domain of an IgA1 IgA2, IgD, IgE, IgM, IgG1, IgG2, IgG3, or IgG4.

According to embodiment 60, an antibody is provided that competes for binding to SLIT2 with the antibody of any of embodiments 46-59.

According to embodiment 61, a pharmaceutical composition is provided comprising the antibody according to any one of embodiments 46-60 and a pharmaceutically acceptable vehicle.

According to embodiment 62, an isolated nucleic acid molecule is provided, comprising one or more nucleotide sequences encoding the antibody of any of embodiments 46-60.

According to embodiment 63, a vector is provided comprising the nucleic acid molecule of embodiment 62.

According to embodiment 64, a host cell is provided comprising the nucleic acid molecule of embodiment 62.

According to embodiment 65, a host cell is provided comprising the vector of embodiment 63.

According to embodiment 66, a method of making the antibody of any one of embodiments 46-60 is provided, comprising expressing the antibody in a host cell and isolating the antibody from the host cell.

According to embodiment 67, a method of treating a renal disease is provided, comprising administering to a subject in need thereof a therapeutically effective amount of the antibody of any of embodiments 46-60, or the pharmaceutical composition of embodiment 61.

According to embodiment 68, the method of embodiment 67 is provided, wherein the renal disease is a glomerular disease, Focal Segmental Glomerular Sclerosis (FSGS), or nephropathy.

According to embodiment 69, a method of reducing proteinuria is provided, comprising administering to a subject in need thereof a therapeutically effective amount of the antibody of any of embodiments 46-60, or the pharmaceutical composition of embodiment 61.

According to embodiment 70, the method of embodiment 69 is provided, wherein the subject is afflicted with a renal disease, wherein said renal disease is a glomerular disease, Focal Segmental Glomerular Sclerosis (FSGS), or nephropathy.

According to embodiment 62, the method of embodiment 69 is provided, wherein said subject is susceptible to a renal disease, wherein said renal disease is a glomerular disease, Focal Segmental Glomerular Sclerosis (FSGS), or nephropathy.

According to embodiment 72, a method of preserving podocyte function is provided, comprising administering to a subject in need thereof a therapeutically effective amount of the antibody of any of embodiments 46-60, or the pharmaceutical composition of embodiment 61.

According to embodiment 73, the method of embodiment 72 is provided, wherein the subject is afflicted with a renal disease, wherein said renal disease is a glomerular disease, Focal Segmental Glomerular Sclerosis (FSGS), or nephropathy.

According to embodiment 74, the method of embodiment 72 is provided, wherein the subject is susceptible to a renal disease, wherein said renal disease is a glomerular disease, Focal Segmental Glomerular Sclerosis (FSGS), or nephropathy.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

Citation of the above publications or documents is not intended as an admission that any of the foregoing is pertinent prior art, nor does it constitute any admission as to the contents or date of these publications or documents.

We claim:

1. An antibody comprising:
a heavy chain comprising complementarity determining regions (CDR) CDR-H1 comprising amino acid sequence of SEQ ID NO: 1; CDR-H2 comprising amino acid sequence of SEQ ID NO: 2; and CDR-H3 comprising amino acid sequence of SEQ ID NO: 3, respectively and having a specific binding activity to human SLIT2 and/or blocking activity to ROBO2; and
a light chain comprising CDR-L1 comprising amino acid sequence of SEQ ID NO: 4 or 7; CDR-L2 comprising amino acid sequence of SEQ ID NO: 5; and CDR-L3 comprising amino acid sequence of SEQ ID NO: 6 and having a specific binding activity to human SLIT2 and/or blocking activity to ROBO2.

2. The antibody of claim 1, wherein the antibody is a chimeric antibody, human antibody or humanized antibody.

3.

4.

5. The antibody of claim 1, wherein the antibody comprises:
a heavy chain variable domain comprising an amino acid sequence at least 85% identical to any of the amino acid sequence of SEQ ID NOS: 8, 24-27, wherein the heavy chain variable domain includes CDR-H1, CDR-H2 and CDR-H3 defined by SEQ ID NOS:1-3, respectively; and
a) a light chain variable domain comprising an amino acid sequence at least 85% identical to any of the amino acid sequence of SEQ ID NOS: 9, 28-29, wherein the light chain variable domain includes CDR-L1, CDR-L2 and CDR-L3 defined by SEQ ID NOS:4-6, respectively, or
b) a light chain variable domain comprising an amino acid sequence at least 85% identical to any of the amino acid sequence of SEQ ID NOS: 30-31, wherein the light chain variable domain includes CDR-L1, CDR-L2 and CDR-L3 defined by SEQ ID NOS:7, 5 and 6, respectively.

6.

7.

8.

9.

10.

11.

12.

13.

14. The antibody of claim 5, wherein the antibody is a chimeric antibody, human antibody or humanized antibody.

15.

16.

17.

18. A recombinant antibody capable of modulating ROBO2 and SLIT2 interaction, wherein the antibody comprises:
a heavy chain comprising an amino acid sequence at least 85% identical to any of the amino acid sequences of SEQ ID NOS: 12, 44-47 and having a specific binding activity to human SLIT2 and/or blocking activity to ROBO2, wherein the heavy chain includes CDR-H1, CDR-H2 and CDR-H3 defined by SEQ ID NOS-3, respectively; and
a) a light chain comprising an amino acid sequence at least 85% identical to any of the amino acid sequences of SEQ ID NO: 13, 48-49 and having a specific binding activity to human SLIT2 and/or blocking activity to ROBO2, wherein the light chain variable domain includes CDR-L1, CDR-L2 and CDR-L3 defined by SEQ ID NOS:4-6, respectively, or
b) a light chain comprising an amino acid sequence at least 85% identical to any of the amino acid sequences of SEQ ID NOS: 50-51 and having a specific binding activity to human SLIT2 and/or blocking activity to ROBO2, wherein the light chain variable domain includes CDR-L1, CDR-L2 and CDR-L3 defined by SEQ ID NOS:7, 5 and 6, respectively.

19.

20.

21.

22.

23. The antibody of claim 1, comprising an Fc domain, wherein the Fc domain is the Fc domain of an IgA1 IgA2, IgD, IgE, IgM, IgG1, IgG2, IgG3, or IgG4.

24. An antibody that competes for binding to SLIT2 with the antibody of claim 1.

25. An isolated nucleic acid molecule, comprising one or more nucleotide sequences encoding the antibody of any of claim 1.

26. A vector or host cell comprising a nucleic acid molecule of claim 25.

27. An isolated nucleic acid molecule, comprising one or more nucleotide sequences encoding the antibody of claim 5

28. A vector or host cell comprising a nucleic acid molecule of claim 27.

29. A method of making the antibody of claim 1, comprising expressing the antibody in a host cell and isolating the antibody from the host cell.

30. A method of treating a renal disease, proteinuria or preserving podocyte function, comprising administering to a subject in need thereof a therapeutically effective amount of the antibody of claim 1.

31.

32.

33. The method of claim 30, wherein the subject is afflicted with a renal disease, wherein said renal disease is a glomerular disease, Focal Segmental Glomerular Sclerosis (FSGS), or nephropathy.

34.

35.

36.

37.

38. An isolated nucleic acid molecule encoding variable regions of an antibody, comprising:
a nucleotide sequence encoding a heavy chain variable region of any of SEQ ID NOS: 10, 32-35; and
a nucleotide sequence encoding a light chain variable region of any of SEQ ID NOS: 11, 36-39.

39. An isolated nucleic acid molecule encoding antibody heavy and light chains, comprising:
a nucleotide sequence encoding a heavy chain of any of SEQ ID NOS: 14, 52-55; and
a nucleotide sequence encoding a light chain of any of SEQ ID NOS: 15, 56-59.

55. The antibody of claim 5, wherein the antibody has a specific binding activity to human SLIT2 and/or blocking activity to ROBO2.

58. The antibody of claim 55, wherein at least a portion of the SLIT2 has the amino acid sequence of SEQ ID NO: 16.

75. The antibody of claim 5, comprising an Fc domain, wherein the Fc domain is the Fc domain of an IgA1 IgA2, IgD, IgE, IgM, IgG1, IgG2, IgG3, or IgG4.

76. The antibody of claim 18, comprising an Fc domain, wherein the Fc domain is the Fc domain of an IgA1 IgA2, IgD, IgE, IgM, IgG1, IgG2, IgG3, or IgG4.

77. An isolated nucleic acid molecule, comprising one or more nucleotide sequences encoding the antibody of any of claim 18

78. A vector or host cell comprising a nucleic acid molecule of claim 77.

79. A method of making the antibody of claim 5, comprising expressing the antibody in a host cell and isolating the antibody from the host cell.

80. A method of making the antibody of claim 18, comprising expressing the antibody in a host cell and isolating the antibody from the host cell.

81. A method of treating a renal disease, proteinuria or preserving podocyte function, comprising administering to a subject in need thereof a therapeutically effective amount of the antibody of claim 5.

82. The method of claim 81, wherein the subject is afflicted with a renal disease, wherein said renal disease is a glomerular disease, Focal Segmental Glomerular Sclerosis (FSGS), or nephropathy.

83. A method of treating a renal disease, proteinuria or preserving podocyte function, comprising administering to a subject in need thereof a therapeutically effective amount of the antibody of claim 18.

84. The method of claim 83, wherein the subject is afflicted with a renal disease, wherein said renal disease is a glomerular disease, Focal Segmental Glomerular Sclerosis (FSGS), or nephropathy.

85. An antibody that competes for binding to SLIT2 with the antibody of claim 5.

86. An antibody that competes for binding to SLIT2 with the antibody of claim 18.

87. The antibody of claim 18, wherein the antibody is a chimeric antibody, human antibody or humanized antibody.


SEQUENCE LISTING (59 sequences)

SEQ ID NO: 1 - protein, 7 aa, synthetic construct
  REGION 1..7: note = Amino acid sequence of 75C1 HCDR1
GGSFRSY

SEQ ID NO: 2 - protein, 6 aa, synthetic construct
  REGION 1..6: note = Amino acid sequence of 75C1 HCDR2
IPIFGT

SEQ ID NO: 3 - protein, 9 aa, synthetic construct
  REGION 1..9: note = Amino acid sequence of 75C1 HCDR3
GSGWYTFDY

SEQ ID NO: 4 - protein, 11 aa, synthetic construct
  REGION 1..11: note = Amino acid sequence of 75C1 LCDR1
RASQNINSYLN

SEQ ID NO: 5 - protein, 7 aa, synthetic construct
  REGION 1..7: note = Amino acid sequence of 75C1 LCDR2
AASNLQS

SEQ ID NO: 6 - protein, 9 aa, synthetic construct
  REGION 1..9: note = Amino acid sequence of 75C1 LCDR3
QQSYRSPPT

SEQ ID NO: 7 - protein, 11 aa, synthetic construct
  REGION 1..11: note = Amino acid sequence of 75C1 LCDR1 substitution mutant
RASQNINAYLN

SEQ ID NO: 8 - protein, 118 aa, synthetic construct
  REGION 1..118: note = Amino acid sequence of 75C1 VH
QVQLVQSGAEVKNPGSSVKVSCKASGGSFRSYTVSWVRQAPGQGLEWMGGIIPIFGTANYAQKFQGRVTITADESTDTTYMDLSSLKSENTAVYYCATGSGWYTFDYWGQGTLVTVSS

SEQ ID NO: 9 - protein, 107 aa, synthetic construct
  REGION 1..107: note = Amino acid sequence of 75C1 VL
DIQMTQSPSSLSASIGDRVTITCRASQNINSYLNWYQQKPGKAPKFLIYAASNLQSGVPSRFSGSGSGTDFNLNISSLQPEDFAIYYCQQSYRSPPTFGGGTTVEIK

SEQ ID NO: 10 - DNA, 354 bp, synthetic construct
  misc_feature 1..354: note = Nucleic acid sequence of 75C1 VH
  source 1..354: mol_type = other DNA
caggtgcagctggtgcagtctggggctgaggtgaagaaccctgggtcctcggtgaaggtctcctgcaaggcttctggaggctccttcaggagctatactgtcagttgggtgcgacaggcccctggacaagggcttgagtggatgggagggatcatccctatctttggtacagcaaactacgcacagaagttccagggcagagtcacgattaccgcggacgaatccacggacacaacctacatggacctgagcagcctgaaatctgaaaacacggccgtgtattattgtgcgacaggtagtggctggtacacctttgactactggggccagggaaccctggtcaccgtctcctca

SEQ ID NO: 11 - DNA, 321 bp, synthetic construct
  misc_feature 1..321: note = Nucleic acid sequence of 75C1 VL
  source 1..321: mol_type = other DNA
gacatccagatgacccagtctccatcctccctgtctgcatctataggagacagagtcaccatcacttgccgggcgagtcaaaacattaacagctatttaaattggtatcagcagaaaccagggaaagcccctaagttcctgatctatgctgcatccaatttgcaaagtggggtcccatcaaggttcagtggcagtggatctgggacagatttcaatctcaacatcagcagtctgcagcctgaagattttgcaatttactactgtcaacagagctatagaagtcctcccactttcggcggagggaccacggtggagattaaa

SEQ ID NO: 12 - protein, 448 aa, synthetic construct
  REGION 1..448: note = Amino acid sequence of 75C1 heavy chain
QVQLVQSGAEVKNPGSSVKVSCKASGGSFRSYTVSWVRQAPGQGLEWMGGIIPIFGTANYAQKFQGRVTITADESTDTTYMDLSSLKSENTAVYYCATGSGWYTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 214 aa, synthetic construct
  REGION 1..214: note = Amino acid sequence of 75C1 light chain
DIQMTQSPSSLSASIGDRVTITCRASQNINSYLNWYQQKPGKAPKFLIYAASNLQSGVPSRFSGSGSGTDFNLNISSLQPEDFAIYYCQQSYRSPPTFGGGTTVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - DNA, 1344 bp, synthetic construct
  misc_feature 1..1344: note = Nucleic acid sequence of 75C1 heavy chain
  source 1..1344: mol_type = other DNA
caggtgcagctggtgcagtctggggctgaggtgaagaaccctgggtcctcggtgaaggtctcctgcaaggcttctggaggctccttcaggagctatactgtcagttgggtgcgacaggcccctggacaagggcttgagtggatgggagggatcatccctatctttggtacagcaaactacgcacagaagttccagggcagagtcacgattaccgcggacgaatccacggacacaacctacatggacctgagcagcctgaaatctgaaaacacggccgtgtattattgtgcgacaggtagtggctggtacacctttgactactggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtacaccctgcccccatcccgggatgagctaaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 15 - DNA, 642 bp, synthetic construct
  misc_feature 1..642: note = Nucleic acid sequence of 75C1 light chain
  source 1..642: mol_type = other DNA
gacatccagatgacccagtctccatcctccctgtctgcatctataggagacagagtcaccatcacttgccgggcgagtcaaaacattaacagctatttaaattggtatcagcagaaaccagggaaagcccctaagttcctgatctatgctgcatccaatttgcaaagtggggtcccatcaaggttcagtggcagtggatctgggacagatttcaatctcaacatcagcagtctgcagcctgaagattttgcaatttactactgtcaacagagctatagaagtcctcccactttcggcggagggaccacggtggagattaaacgtacggtggcggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 16 - protein, 1099 aa, synthetic construct
  REGION 1..1099: note = SLIT2: O94813(26-1118)-his
QACPAQCSCSGSTVDCHGLALRSVPRNIPRNTERLDLNGNNITRITKTDFAGLRHLRVLQLMENKISTIERGAFQDLKELERLRLNRNHLQLFPELLFLGTAKLYRLDLSENQIQAIPRKAFRGAVDIKNLQLDYNQISCIEDGAFRALRDLEVLTLNNNNITRLSVASFNHMPKLRTFRLHSNNLYCDCHLAWLSDWLRQRPRVGLYTQCMGPSHLRGHNVAEVQKREFVCSGHQSFMAPSCSVLHCPAACTCSNNIVDCRGKGLTEIPTNLPETITEIRLEQNTIKVIPPGAFSPYKKLRRIDLSNNQISELAPDAFQGLRSLNSLVLYGNKITELPKSLFEGLFSLQLLLLNANKINCLRVDAFQDLHNLNLLSLYDNKLQTIAKGTFSPLRAIQTMHLAQNPFICDCHLKWLADYLHTNPIETSGARCTSPRRLANKRIGQIKSKKFRCSAKEQYFIPGTEDYRSKLSGDCFADLACPEKCRCEGTTVDCSNQKLNKIPEHIPQYTAELRLNNNEFTVLEATGIFKKLPQLRKINFSNNKITDIEEGAFEGASGVNEILLTSNRLENVQHKMFKGLESLKTLMLRSNRITCVGNDSFIGLSSVRLLSLYDNQITTVAPGAFDTLHSLSTLNLLANPFNCNCYLAWLGEWLRKKRIVTGNPRCQKPYFLKEIPIQDVAIQDFTCDDGNDDNSCSPLSRCPTECTCLDTVVRCSNKGLKVLPKGIPRDVTELYLDGNQFTLVPKELSNYKHLTLIDLSNNRISTLSNQSFSNMTQLLTLILSYNRLRCIPPRTFDGLKSLRLLSLHGNDISVVPEGAFNDLSALSHLAIGANPLYCDCNMQWLSDWVKSEYKEPGIARCAGPGEMADKLLLTTPSKKFTCQGPVDVNILAKCNPCLSNPCKNDGTCNSDPVDFYRCTCPYGFKGQDCDVPIHACISNPCKHGGTCHLKEGEEDGFWCICADGFEGENCEVNVDDCEDNDCENNSTCVDGINNYTCLCPPEYTGELCEEKLDFCAQDLNPCQHDSKCILTPKGFKCDCTPGYVGEHCDIDFDDCQDNKCKNGAHCTDAVNGYTCICPEGYSGLFCEFSPPMVHHHHHH

SEQ ID NO: 17 - DNA, 21 bp, synthetic construct
  misc_feature 1..21: note = Nucleic acid sequence of 75C1 HCDR1
  source 1..21: mol_type = other DNA
ggaggctccttcaggagctat

SEQ ID NO: 18 - DNA, 18 bp, synthetic construct
  misc_feature 1..18: note = Nucleic acid sequence of 75C1 HCDR2
  source 1..18: mol_type = other DNA
atccctatctttggtaca

SEQ ID NO: 19 - DNA, 27 bp, synthetic construct
  misc_feature 1..27: note = Nucleic acid sequence of 75C1 HCDR3
  source 1..27: mol_type = other DNA
ggtagtggctggtacacctttgactac

SEQ ID NO: 20 - DNA, 33 bp, synthetic construct
  misc_feature 1..33: note = Nucleic acid sequence of 75C1 LCDR1
  source 1..33: mol_type = other DNA
cgggcgagtcaaaacattaacagctatttaaat

SEQ ID NO: 21 - DNA, 21 bp, synthetic construct
  misc_feature 1..21: note = Nucleic acid sequence of 75C1 LCDR2
  source 1..21: mol_type = other DNA
gctgcatccaatttgcaaagt

SEQ ID NO: 22 - DNA, 27 bp, synthetic construct
  misc_feature 1..27: note = Nucleic acid sequence of 75C1 LCDR3
  source 1..27: mol_type = other DNA
caacagagctatagaagtcctcccact

SEQ ID NO: 23 - DNA, 33 bp, synthetic construct
  misc_feature 1..33: note = Nucleic acid sequence of 75C1 LCDR1 substitution mutant
  source 1..33: mol_type = other DNA
cgggcgagtcaaaacattaacgcgtatttaaat

SEQ ID NO: 24 - protein, 118 aa, synthetic construct
  REGION 1..118: note = Amino acid sequence of 75C1_HC_MUT_1.1 VH
QVQLVQSGAEVKQPGSSVKVSCKASGGSFRSYTVSWVRQAPGQGLEWMGGIIPIFGTANYAQKFQGRVTITADESTDTTYMDLSSLKSENTAVYYCATGSGWYTFDYWGQGTLVTVSS

SEQ ID NO: 25 - protein, 118 aa, synthetic construct
  REGION 1..118: note = Amino acid sequence of 75C1_HC_MUT_1.2 VH
QVQLVQSGAEVKQPGSSVKVSCKASGGSFRSYTVSWVRQAPGQGLEWMGGIIPIFGTANYAQKFQGRVTITADESTDTVYMDLSSLKSENTAVYYCATGSGWYTFDYWGQGTLVTVSS

SEQ ID NO: 26 - protein, 118 aa, synthetic construct
  REGION 1..118: note = Amino acid sequence of 75C1_HC_MUT_1.3 VH
QVQLVQSGAEVKQPGSSVKVSCKASGGSFRSYTVSWVRQAPGQGLEWMGGIIPIFGTANYAQKFQGRVTITADESTDTVYMDLSSLKSEDTAVYYCATGSGWYTFDYWGQGTLVTVSS

SEQ ID NO: 27 - protein, 118 aa, synthetic construct
  REGION 1..118: note = Amino acid sequence of 75C1_HC_MUT_1.4 VH
QVQLVQSGAEVKQPGSSVKVSCKASGGSFRSYTVSWVRQAPGQGLEWMGGIIPIFGTANYAQKFQGRVTITADESTDTTYMDLSSLKSEDTAVYYCATGSGWYTFDYWGQGTLVTVSS

SEQ ID NO: 28 - protein, 107 aa, synthetic construct
  REGION 1..107: note = Amino acid sequence of 75C1_LC_MUT_1.1 VL
DIQMTQSPSSLSASIGDRVTITCRASQNINSYLNWYQQKPGKAPKFLIYAASNLQSGVPSRFSGSGSGTDFTLTISSLQPEDFAIYYCQQSYRSPPTFGGGTTVEIK

SEQ ID NO: 29 - protein, 107 aa, synthetic construct
  REGION 1..107: note = Amino acid sequence of 75C1_LC_MUT_1.2 VL
DIQMTQSPSSLSASIGDRVTITCRASQNINSYLNWYQQKPGKAPKFLIYAASNLQSGVPSRFSGSGSGTDFTLNISSLQPEDFAIYYCQQSYRSPPTFGGGTTVEIK

SEQ ID NO: 30 - protein, 107 aa, synthetic construct
  REGION 1..107: note = Amino acid sequence of 75C1_LC_MUT_2.1 VL
DIQMTQSPSSLSASIGDRVTITCRASQNINAYLNWYQQKPGKAPKFLIYAASNLQSGVPSRFSGSGSGTDFTLTISSLQPEDFAIYYCQQSYRSPPTFGGGTTVEIK

SEQ ID NO: 31 - protein, 107 aa, synthetic construct
  REGION 1..107: note = Amino acid sequence of 75C1_LC_MUT_2.2 VL
DIQMTQSPSSLSASIGDRVTITCRASQNINAYLNWYQQKPGKAPKFLIYAASNLQSGVPSRFSGSGSGTDFTLNISSLQPEDFAIYYCQQSYRSPPTFGGGTTVEIK

SEQ ID NO: 32 - DNA, 354 bp, synthetic construct
  misc_feature 1..354: note = Nucleic acid sequence of 75C1_HC_MUT_1.1 VH
  source 1..354: mol_type = other DNA
caggtgcagctggtgcagtctggggctgaggtgaagcaacctgggtcctcggtgaaggtctcctgcaaggcttctggaggctccttcaggagctatactgtcagttgggtgcgacaggcccctggacaagggcttgagtggatgggagggatcatccctatctttggtacagcaaactacgcacagaagttccagggcagagtcacgattaccgcggacgaatccacggacacaacctacatggacctgagcagcctgaaatctgaaaacacggccgtgtattattgtgcgacaggtagtggctggtacacctttgactactggggccagggaaccctggtcaccgtctcctca

SEQ ID NO: 33 - DNA, 354 bp, synthetic construct
  misc_feature 1..354: note = Nucleic acid sequence of 75C1_HC_MUT_1.2 VH
  source 1..354: mol_type = other DNA
caggtgcagctggtgcagtctggggctgaggtgaagcaacctgggtcctcggtgaaggtctcctgcaaggcttctggaggctccttcaggagctatactgtcagttgggtgcgacaggcccctggacaagggcttgagtggatgggagggatcatccctatctttggtacagcaaactacgcacagaagttccagggcagagtcacgattaccgcggacgaatccacggacacagtatacatggacctgagcagcctgaaatctgaaaacacggccgtgtattattgtgcgacaggtagtggctggtacacctttgactactggggccagggaaccctggtcaccgtctcctca

SEQ ID NO: 34 - DNA, 354 bp, synthetic construct
  misc_feature 1..354: note = Nucleic acid sequence of 75C1_HC_MUT_1.3 VH
  source 1..354: mol_type = other DNA
caggtgcagctggtgcagtctggggctgaggtgaagcaacctgggtcctcggtgaaggtctcctgcaaggcttctggaggctccttcaggagctatactgtcagttgggtgcgacaggcccctggacaagggcttgagtggatgggagggatcatccctatctttggtacagcaaactacgcacagaagttccagggcagagtcacgattaccgcggacgaatccacggacacagtatacatggacctgagcagcctgaaatctgaagatacggccgtgtattattgtgcgacaggtagtggctggtacacctttgactactggggccagggaaccctggtcaccgtctcctca

SEQ ID NO: 35 - DNA, 354 bp, synthetic construct
  misc_feature 1..354: note = Nucleic acid sequence of 75C1_HC_MUT_1.4 VH
  source 1..354: mol_type = other DNA
caggtgcagctggtgcagtctggggctgaggtgaagcaacctgggtcctcggtgaaggtctcctgcaaggcttctggaggctccttcaggagctatactgtcagttgggtgcgacaggcccctggacaagggcttgagtggatgggagggatcatccctatctttggtacagcaaactacgcacagaagttccagggcagagtcacgattaccgcggacgaatccacggacacaacctacatggacctgagcagcctgaaatctgaagacacggccgtgtattattgtgcgacaggtagtggctggtacacctttgactactggggccagggaaccctggtcaccgtctcctca

SEQ ID NO: 36 - DNA, 321 bp, synthetic construct
  misc_feature 1..321: note = Nucleic acid sequence of 75C1_LC_MUT_1.1 VL
  source 1..321: mol_type = other DNA
gacatccagatgacccagtctccatcctccctgtctgcatctataggagacagagtcaccatcacttgccgggcgagtcaaaacattaacagctatttaaattggtatcagcagaaaccagggaaagcccctaagttcctgatctatgctgcatccaatttgcaaagtggggtcccatcaaggttcagtggcagtggatctgggacagatttcacactcactatcagcagtctgcagcctgaagattttgcaatttactactgtcaacagagctatagaagtcctcccactttcggcggagggaccacggtggagattaaa

SEQ ID NO: 37 - DNA, 321 bp, synthetic construct
  misc_feature 1..321: note = Nucleic acid sequence of 75C1_LC_MUT_1.2 VL
  source 1..321: mol_type = other DNA
gacatccagatgacccagtctccatcctccctgtctgcatctataggagacagagtcaccatcacttgccgggcgagtcaaaacattaacagctatttaaattggtatcagcagaaaccagggaaagcccctaagttcctgatctatgctgcatccaatttgcaaagtggggtcccatcaaggttcagtggcagtggatctgggacagatttcacactcaacatcagcagtctgcagcctgaagattttgcaatttactactgtcaacagagctatagaagtcctcccactttcggcggagggaccacggtggagattaaa

SEQ ID NO: 38 - DNA, 321 bp, synthetic construct
  misc_feature 1..321: note = Nucleic acid sequence of 75C1_LC_MUT_2.1 VL
  source 1..321: mol_type = other DNA
gacatccagatgacccagtctccatcctccctgtctgcatctataggagacagagtcaccatcacttgccgggcgagtcaaaacattaacgcgtatttaaattggtatcagcagaaaccagggaaagcccctaagttcctgatctatgctgcatccaatttgcaaagtggggtcccatcaaggttcagtggcagtggatctgggacagatttcacactcactatcagcagtctgcagcctgaagattttgcaatttactactgtcaacagagctatagaagtcctcccactttcggcggagggaccacggtggagattaaa

SEQ ID NO: 39 - DNA, 321 bp, synthetic construct
  misc_feature 1..321: note = Nucleic acid sequence of 75C1_LC_MUT_2.2 VL
  source 1..321: mol_type = other DNA
gacatccagatgacccagtctccatcctccctgtctgcatctataggagacagagtcaccatcacttgccgggcgagtcaaaacattaacgcgtatttaaattggtatcagcagaaaccagggaaagcccctaagttcctgatctatgctgcatccaatttgcaaagtggggtcccatcaaggttcagtggcagtggatctgggacagatttcacactcaacatcagcagtctgcagcctgaagattttgcaatttactactgtcaacagagctatagaagtcctcccactttcggcggagggaccacggtggagattaaa

SEQ ID NO: 40 - protein, 330 aa, synthetic construct
  REGION 1..330: note = Amino acid sequence of heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 107 aa, synthetic construct [sequence omitted]

SEQ ID NO: 42 - DNA, 990 bp, synthetic construct
  misc_feature 1..990: note = Nucleotide sequence of heavy chain constant region
  source 1..990: mol_type = other DNA
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtacaccctgcccccatcccgggatgagctaaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 43 - DNA, 321 bp, synthetic construct
  misc_feature 1..321: note = Nucleotide sequence of light chain constant region
  source 1..321: mol_type = other DNA
cgtacggtggcggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 44 - protein, 448 aa, synthetic construct
  REGION 1..448: note = Amino acid sequence of 75C1_HC_MUT_1.1 heavy chain
QVQLVQSGAEVKQPGSSVKVSCKASGGSFRSYTVSWVRQAPGQGLEWMGGIIPIFGTANYAQKFQGRVTITADESTDTTYMDLSSLKSENTAVYYCATGSGWYTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 448 aa, synthetic construct
  REGION 1..448: note = Amino acid sequence of 75C1_HC_MUT_1.2 heavy chain
QVQLVQSGAEVKQPGSSVKVSCKASGGSFRSYTVSWVRQAPGQGLEWMGGIIPIFGTANYAQKFQGRVTITADESTDTVYMDLSSLKSENTAVYYCATGSGWYTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 448 aa, synthetic construct
  REGION 1..448: note = Amino acid sequence of 75C1_HC_MUT_1.3 heavy chain
QVQLVQSGAEVKQPGSSVKVSCKASGGSFRSYTVSWVRQAPGQGLEWMGGIIPIFGTANYAQKFQGRVTITADESTDTVYMDLSSLKSEDTAVYYCATGSGWYTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 448 aa, synthetic construct
  REGION 1..448: note = Amino acid sequence of 75C1_HC_MUT_1.4 heavy chain
QVQLVQSGAEVKQPGSSVKVSCKASGGSFRSYTVSWVRQAPGQGLEWMGGIIPIFGTANYAQKFQGRVTITADESTDTTYMDLSSLKSEDTAVYYCATGSGWYTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 214 aa, synthetic construct
  REGION 1..214: note = Amino acid sequence of 75C1_LC_MUT_1.1 light chain
DIQMTQSPSSLSASIGDRVTITCRASQNINSYLNWYQQKPGKAPKFLIYAASNLQSGVPSRFSGSGSGTDFTLTISSLQPEDFAIYYCQQSYRSPPTFGGGTTVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 214 aa, synthetic construct
  REGION 1..214: note = Amino acid sequence of 75C1_LC_MUT_1.2 light chain
DIQMTQSPSSLSASIGDRVTITCRASQNINSYLNWYQQKPGKAPKFLIYAASNLQSGVPSRFSGSGSGTDFTLNISSLQPEDFAIYYCQQSYRSPPTFGGGTTVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 214 aa, synthetic construct
  REGION 1..214: note = Amino acid sequence of 75C1_LC_MUT_2.1 light chain
DIQMTQSPSSLSASIGDRVTITCRASQNINAYLNWYQQKPGKAPKFLIYAASNLQSGVPSRFSGSGSGTDFTLTISSLQPEDFAIYYCQQSYRSPPTFGGGTTVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 214 aa, synthetic construct
  REGION 1..214: note = Amino acid sequence of 75C1_LC_MUT_2.2 light chain
DIQMTQSPSSLSASIGDRVTITCRASQNINAYLNWYQQKPGKAPKFLIYAASNLQSGVPSRFSGSGSGTDFTLNISSLQPEDFAIYYCQQSYRSPPTFGGGTTVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - DNA, 1344 bp, synthetic construct
  misc_feature 1..1344: note = Nucleic acid sequence of 75C1_HC_MUT_1.1 heavy chain
  source 1..1344: mol_type = other DNA
caggtgcagctggtgcagtctggggctgaggtgaagcaacctgggtcctcggtgaaggtctcctgcaaggcttctggaggctccttcaggagctatactgtcagttgggtgcgacaggcccctggacaagggcttgagtggatgggagggatcatccctatctttggtacagcaaactacgcacagaagttccagggcagagtcacgattaccgcggacgaatccacggacacaacctacatggacctgagcagcctgaaatctgaaaacacggccgtgtattattgtgcgacaggtagtggctggtacacctttgactactggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtacaccctgcccccatcccgggatgagctaaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 53 - DNA, 1344 bp, synthetic construct
  misc_feature 1..1344: note = Nucleic acid sequence of 75C1_HC_MUT_1.2 heavy chain
  source 1..1344: mol_type = other DNA
caggtgcagctggtgcagtctggggctgaggtgaagcaacctgggtcctcggtgaaggtctcctgcaaggcttctggaggctccttcaggagctatactgtcagttgggtgcgacaggcccctggacaagggcttgagtggatgggagggatcatccctatctttggtacagcaaactacgcacagaagttccagggcagagtcacgattaccgcggacgaatccacggacacagtatacatggacctgagcagcctgaaatctgaaaacacggccgtgtattattgtgcgacaggtagtggctggtacacctttgactactggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtacaccctgcccccatcccgggatgagctaaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 54 - DNA, 1344 bp, synthetic construct
  misc_feature 1..1344: note = Nucleic acid sequence of 75C1_HC_MUT_1.3 heavy chain
  source 1..1344: mol_type = other DNA
caggtgcagctggtgcagtctggggctgaggtgaagcaacctgggtcctcggtgaaggtctcctgcaaggcttctggaggctccttcaggagctatactgtcagttgggtgcgacaggcccctggacaagggcttgagtggatgggagggatcatccctatctttggtacagcaaactacgcacagaagttccagggcagagtcacgattaccgcggacgaatccacggacacagtatacatggacctgagcagcctgaaatctgaagatacggccgtgtattattgtgcgacaggtagtggctggtacacctttgactactggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtacaccctgcccccatcccgggatgagctaaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 55 - DNA, 1344 bp, synthetic construct
  misc_feature 1..1344: note = Nucleic acid sequence of 75C1_HC_MUT_1.4 heavy chain
  source 1..1344: mol_type = other DNA
caggtgcagctggtgcagtctggggctgaggtgaagcaacctgggtcctcggtgaaggtctcctgcaaggcttctggaggctccttcaggagctatactgtcagttgggtgcgacaggcccctggacaagggcttgagtggatgggagggatcatccctatctttggtacagcaaactacgcacagaagttccagggcagagtcacgattaccgcggacgaatccacggacacaacctacatggacctgagcagcctgaaatctgaagacacggccgtgtattattgtgcgacaggtagtggctggtacacctttgactactggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtacaccctgcccccatcccgggatgagctaaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 56 - DNA, 642 bp, synthetic construct
  misc_feature 1..642: note = Nucleic acid sequence of 75C1_LC_MUT_1.1 light chain
  source 1..642: mol_type = other DNA
gacatccagatgacccagtctccatcctccctgtctgcatctataggagacagagtcaccatcacttgccgggcgagtcaaaacattaacagctatttaaattggtatcagcagaaaccagggaaagcccctaagttcctgatctatgctgcatccaatttgcaaagtggggtcccatcaaggttcagtggcagtggatctgggacagatttcacactcactatcagcagtctgcagcctgaagattttgcaatttactactgtcaacagagctatagaagtcctcccactttcggcggagggaccacggtggagattaaacgtacggtggcggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 57 - DNA, 642 bp, synthetic construct
  misc_feature 1..642: note = Nucleic acid sequence of 75C1_LC_MUT_1.2 light chain
  source 1..642: mol_type = other DNA
gacatccagatgacccagtctccatcctccctgtctgcatctataggagacagagtcaccatcacttgccgggcgagtcaaaacattaacagctatttaaattggtatcagcagaaaccagggaaagcccctaagttcctgatctatgctgcatccaatttgcaaagtggggtcccatcaaggttcagtggcagtggatctgggacagatttcacactcaacatcagcagtctgcagcctgaagattttgcaatttactactgtcaacagagctatagaagtcctcccactttcggcggagggaccacggtggagattaaacgtacggtggcggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 58 - DNA, 642 bp, synthetic construct
  misc_feature 1..642: note = Nucleic acid sequence of 75C1_LC_MUT_2.1 light chain
  source 1..642: mol_type = other DNA
gacatccagatgacccagtctccatcctccctgtctgcatctataggagacagagtcaccatcacttgccgggcgagtcaaaacattaacgcgtatttaaattggtatcagcagaaaccagggaaagcccctaagttcctgatctatgctgcatccaatttgcaaagtggggtcccatcaaggttcagtggcagtggatctgggacagatttcacactcactatcagcagtctgcagcctgaagattttgcaatttactactgtcaacagagctatagaagtcctcccactttcggcggagggaccacggtggagattaaacgtacggtggcggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 59 - DNA, 642 bp, synthetic construct
  misc_feature 1..642: note = Nucleic acid sequence of 75C1_LC_MUT_2.2 light chain
  source 1..642: mol_type = other DNA
gacatccagatgacccagtctccatcctccctgtctgcatctataggagacagagtcaccatcacttgccgggcgagtcaaaacattaacgcgtatttaaattggtatcagcagaaaccagggaaagcccctaagttcctgatctatgctgcatccaatttgcaaagtggggtcccatcaaggttcagtggcagtggatctgggacagatttcacactcaacatcagcagtctgcagcctgaagattttgcaatttactactgtcaacagagctatagaagtcctcccactttcggcggagggaccacggtggagattaaacgtacggtggcggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt